SEQUENCE LISTING (16 sequences)

SEQ ID NO: 1 - protein, 5 aa, synthetic construct
  REGION 1..5: note = Synthetic Construct
DYYIT

SEQ ID NO: 2 - protein, 17 aa, synthetic construct
  REGION 1..17: note = Synthetic Construct
WIYPGSGNTKYNEKFKG

SEQ ID NO: 3 - protein, 8 aa, synthetic construct
  REGION 1..8: note = Synthetic Construct
YGNYWFAY

SEQ ID NO: 4 - protein, 15 aa, synthetic construct
  REGION 1..15: note = Synthetic Construct
KASQSVDFDGDSYMN

SEQ ID NO: 5 - protein, 7 aa, synthetic construct
  REGION 1..7: note = Synthetic Construct
AASNLES

SEQ ID NO: 6 - protein, 9 aa, synthetic construct
  REGION 1..9: note = Synthetic Construct
QQSNEDPWT

SEQ ID NO: 7 - protein, 117 aa, synthetic construct
  REGION 1..117: note = Synthetic Construct
QIQLQQSGPEVVKPGASVKISCKASGYTFTDYYITWVKQKPGQGLEWIGWIYPGSGNTKYNEKFKGKATLTVDTSSSTAFMQLSSLTSEDTAVYFCANYGNYWFAYWGQGTQVTVSA

SEQ ID NO: 8 - protein, 111 aa, synthetic construct
  REGION 1..111: note = Synthetic Construct
DIVLTQSPASLAVSLGQRATISCKASQSVDFDGDSYMNWYQQKPGQPPKVLIYAASNLESGIPARFSGSGSGTDFTLNIHPVEEEDAATYYCQQSNEDPWTFGGGTKLEIK

SEQ ID NO: 9 - protein, 30 aa, synthetic construct
  REGION 1..30: note = Synthetic Construct
QIQLQQSGPEVVKPGASVKISCKASGYTFT

SEQ ID NO: 10 - protein, 14 aa, synthetic construct
  REGION 1..14: note = Synthetic Construct
WVKQKPGQGLEWIG

SEQ ID NO: 11 - protein, 32 aa, synthetic construct
  REGION 1..32: note = Synthetic Construct
KATLTVDTSSSTAFMQLSSLTSEDTAVYFCAN

SEQ ID NO: 12 - protein, 11 aa, synthetic construct
  REGION 1..11: note = Synthetic Construct
WGQGTQVTVSA

SEQ ID NO: 13 - protein, 23 aa, synthetic construct
  REGION 1..23: note = Synthetic Construct
DIVLTQSPASLAVSLGQRATISC

SEQ ID NO: 14 - protein, 15 aa, synthetic construct
  REGION 1..15: note = Synthetic Construct
WYQQKPGQPPKVLIY

SEQ ID NO: 15 - protein, 32 aa, synthetic construct
  REGION 1..32: note = Synthetic Construct
GIPARFSGSGSGTDFTLNIHPVEEEDAATYYC

SEQ ID NO: 16 - protein, 10 aa, synthetic construct
  REGION 1..10: note = Synthetic Construct
FGGGTKLEIK

(19) United States
(12) Patent Application Publication
(10) Pub. No.: US 2023/0279133 A1
(43) Pub. Date: Sep. 7, 2023

(54) MODULATING THE IMMUNE RESPONSE USING ANTIBODY-DRUG CONJUGATES

(71) Applicant: Seagen Inc., Bothell, WA (US)

(72) Inventors: Ryan HEISER, Lake Stevens, WA (US); Shyra GARDAI, Bothell, WA (US); David TAFT, Bothell, WA (US); Carol Anne OGDEN, Bothell, WA (US)

(21) Appl. No.: 18/047,240

(22) Filed: Oct. 17, 2022

Related U.S. Application Data
(63) Continuation of application No. 16/755,091, filed on Apr. 9, 2020, now abandoned.
(60) Provisional application No. 62/657,511, filed on Apr. 13, 2018.
(60) Provisional application No. 62/576,017, filed on Oct. 23, 2017.
(60) Provisional application No. 62/572,345, filed on Oct. 13, 2017.

(51) Int. Cl. C07K 16/28; A61K 47/68

(52) U.S. Cl. CPC C07K 16/2878; A61K 47/6803; A61K 47/6849; A61K 2039/507

(57) ABSTRACT

The invention provides methods and compositions for modulating the immune response in a subject, such as decreasing the activity of CD30⁺ T regulatory cells and increasing the ratio of CD8⁺ T cells to CD30⁺ T regulatory cells, through administration of antibody drug-conjugates that bind to CD30. The invention also provides articles of manufacture or kits comprising said antibody drug-conjugates that bind to CD30 for modulating the immune response.

### CROSS-REFERENCE TO RELATED APPLICATIONS

This application claims priority to U.S. Provisional Application No. 62/572,345 filed on Oct. 13, 2017, U.S. Provisional Application No. 62/576,017 filed on Oct. 23, 2017, and U.S. Provisional Application No. 62/657,511 filed on Apr. 13, 2018; the contents of each of which are incorporated herein by reference in their entirety.

### SUBMISSION OF SEQUENCE LISTING ON ASCII TEXT FILE

The content of the following submission on ASCII text file is incorporated herein by reference in its entirety: a computer readable form (CRF) of the Sequence Listing (file name: 761682000140SEQLIST.TXT, date recorded: Oct. 8, 2018, size: 6 KB).

### FIELD OF THE INVENTION

The present invention relates to anti-CD30 antibody-drug conjugates and methods of using the same to modulate the immune response for the treatment of cancer in a subject.

### BACKGROUND OF THE INVENTION

CD30 is a 120 kilodalton membrane glycoprotein (Froese et al., 1987, J. Immunol. 139: 2081-87) and a member of the TNF-receptor superfamily that has been shown to be a marker of malignant cells in Hodgkin's lymphoma and anaplastic large cell lymphoma (ALCL), a subset of non-Hodgkin's lymphoma (NHL) (DUrkop et al., 1992, Cell 88:421-427). CD30 has been found to be highly expressed on the cell surface of all Hodgkin's lymphomas and the majority of ALCL (Josimovic-Alasevic et al., 1989, Eur. J. Immunol. 19:157-162).

CD30 was originally identified by the monoclonal antibody Ki-1 (Schwab et al., 1982, Nature 299:65-67). This monoclonal antibody was developed against Hodgkin and Reed-Sternberg (H-RS) cells, the malignant cells of Hodgkin's lymphoma. A second monoclonal antibody, capable of binding a formalin resistant epitope different from that recognized by Ki-1, was subsequently described (Schwarting et al, 1989 Blood 74:1678-1689). The identification of four additional antibodies resulted in the creation of the CD30 cluster at the Third Leucocyte Typing Workshop in 1986 (McMichael, A, ed., 1987, Leukocyte Typing 111 (Oxford: Oxford University Press)). Monoclonal antibodies specific for the CD30 antigen have been explored as vehicles for the delivery of cytostatic drugs, plant toxins and radioisotopes to cancerous cells expressing CD30 in both preclinical models and clinical studies (Engert et al., 1990, Cancer Research 50:84-88; Barth et al., 2000, Blood 95:3909-3914). In patients with Hodgkin's lymphoma, targeting of the CD30 antigen could be achieved with low doses of the anti-CD30 antibody, BerH2 (Falini et al., 1992, British Journal of Haematology 82:38-45). Yet, despite successful in vivo targeting of the malignant tumor cells, none of the patients experienced tumor regression. In a subsequent clinical trial, the toxin saporin was chemically conjugated to the BerH2 antibody and all four patients demonstrated rapid and substantial reductions in tumor mass (Falini et al., 1992, Lancet 339:1195-1196). However, in vitro studies using an antibody drug-conjugate (ADC) where the toxin dgA was conjugated to the Ki-1 antibody demonstrated only moderate efficacy when administered to patients with resistant HL in a Phase 1 clinical trial (Schnell et al., 2002, Clinical Cancer Research, 8(6):1779-1786).

T regulatory cells (Tregs) are essential modulators of T cell immune responses, limiting chronic inflammation and protecting normal tissues from autoimmunity. T regulatory cells are also implicated in maintaining immune-suppressive conditions in the tumor microenvironment, abrogating cytotoxic anti-tumor immunosurveillance. Analysis of clinical tumor samples has shown increased densities of intratumoral Tregs associated with poor clinical outcomes in a number of cancer types (Fridman, 2012, Nature Reviews Cancer; Charoentong, 2017, Cell Reports 18: 248-262). Recent transcriptomic analyses of intratumoral Tregs isolated from breast, lung, and colorectal cancer tissues showed TNFSFR8 (CD30) to be among transcripts differentially upregulated compared to Tregs isolated from adjacent normal tissue and circulating in blood (Plitas, 2016, Immunity, 45: 1122-1134; De Simone, 2016, Immunity, 45: 1135-1147). The functional significance of heightened CD30 transcript expression in Tregs remains unclear. Given the protective role of Tregs in promoting immune homeostasis in normal tissues, there is considerable interest in developing cancer therapeutics that preferentially target intratumoral Tregs, while sparing those in non-diseased tissues. Therefore, there appears to be a need for therapies that can selectively control the activity of immune cells that are involved in pathogenesis of cancer, such as the activity of T regulatory cells.

All references cited herein, including patent applications, patent publications, and scientific literature, are herein incorporated by reference in their entirety, as if each individual reference were specifically and individually indicated to be incorporated by reference.

### SUMMARY

In one aspect, the present invention provides for a method of decreasing the activity of CD30⁺ T regulatory (Treg) cells in a subject having cancer comprising administering to the subject an antibody-drug conjugate, wherein the antibody-drug conjugate comprises an anti-CD30 antibody or an antigen-binding portion thereof conjugated to a monomethyl auristatin.

In some embodiments, decreasing the activity of CD30⁺ Treg cells comprises a decrease in the number of CD30⁺ Treg cells. In some embodiments, the number of CD30⁺ Treg cells is decreased relative to the number of one or more other types of CD4⁺ T cells. In some embodiments, the one or more other types of CD4⁺ T cells comprise Th1 cells, Th2 cells or Th17 cells. In some embodiments, the one or more other types of CD4⁺ T cells comprise Th1 CD30⁺ cells, Th2 CD30⁺ cells or Th17 CD30⁺ cells. In some embodiments, the number of CD30⁺ Treg cells is decreased relative to the number of CD30⁺ Treg cells in the subject prior to administration of the antibody-drug conjugate.

In some embodiments, decreasing the activity of CD30⁺ Treg cells comprises a decrease in the function of CD30⁺ Treg cells. In some embodiments, the decrease in the function of CD30⁺ Treg cells is relative to the function of CD30⁺ Treg cells in a subject prior to administration of the antibody-drug conjugate.

In some embodiments, the CD30⁺ Treg cells are CD30⁺ inducible T regulatory (iTreg) cells or CD30⁺ peripheral T regulatory (pTreg) cells.

In some embodiments, the monomethyl auristatin is monomethyl auristatin E (MMAE). In some embodiments, the monomethyl auristatin is monomethyl auristatin F (MMAF).

In some embodiments, the anti-CD30 antibody is monoclonal anti-CD30 antibody AC10. In some embodiments, the anti-CD30 antibody is cAC10. In some embodiments, the antibody-drug conjugate is brentuximab vedotin.

In some embodiments, the anti-CD30 antibody comprises a heavy chain variable region and a light chain variable region, wherein the heavy chain variable region comprises:
(i) a CDR-H1 comprising the amino acid sequence of SEQ ID NO: 1;
(ii) a CDR-H2 comprising the amino acid sequence of SEQ ID NO: 2; and
(iii) a CDR-H3 comprising the amino acid sequence of SEQ ID NO: 3; and
   wherein the light chain variable region comprises:
(i) a CDR-L1 comprising the amino acid sequence of SEQ ID NO: 4;
(ii) a CDR-L2 comprising the amino acid sequence of SEQ ID NO: 5; and
(iii) a CDR-L3 comprising the amino acid sequence of SEQ ID NO: 6.

In some embodiments, the anti-CD30 antibody comprises a heavy chain variable region comprising the amino acid sequence of SEQ ID NO: 7 and a light chain variable region comprising the amino acid sequence of SEQ ID NO: 8.

In some embodiments, the antibody-drug conjugate comprises a linker between the anti-CD30 antibody or antigen-binding portion thereof and the monomethyl auristatin. In some embodiments, the linker is selected from the group consisting of a cleavable linker and a non-cleavable linker. In some embodiments, the linker is a cleavable peptide linker. In some embodiments, the linker is a protease-cleavable linker. In some embodiments, the protease cleavable linker is comprises a thiolreactive spacer and a dipeptide. In some embodiments, the protease cleavable linker comprises a thiolreactive maleimidocaproyl spacer, a valine-citrulline dipeptide, and a p-amino-benzyloxycarbonyl spacer. In some embodiments, the cleavable peptide linker has a formula: -MC-vc-PAB-. In some embodiments, the linker is a non-cleavable linker having a formula: -MC-.

In some embodiments, the subject has been previously treated for the cancer. In some embodiments, the subject did not respond to treatment or relapsed after first-line treatment. In some embodiments, the subject has not previously been treated for the cancer.

In some embodiments, the cancer is a lymphoma. In some embodiments, the lymphoma is a T-cell lymphoma. In some embodiments, the lymphoma is a B-cell lymphoma.

In some embodiments, the lymphoma is a non-Hodgkin lymphoma. In some embodiments, the subject has been previously treated for the non-Hodgkin lymphoma and the subject did not respond to treatment or relapsed after first-line treatment. In some embodiments, the subject has not been previously treated for the non-Hodgkin lymphoma. In some embodiments, the non-Hodgkin lymphoma is a mature T-cell lymphoma. In some embodiments, the non-Hodgkin lymphoma is diffuse large B-cell lymphoma (DLBCL), peripheral T-cell lymphoma (PTCL), anaplastic large cell lymphoma (ALCL) or cutaneous T-cell lymphoma (CTCL). In some embodiments, the non-Hodgkin lymphoma is cutaneous T-cell lymphoma (CTCL). In some embodiments, the cutaneous T-cell lymphoma (CTCL) is a mycosis fungoides (MF). In some embodiments, the mycosis fungoides is a CD30-positive mycosis fungoides (MF). In some embodiments, the cutaneous T-cell lymphoma (CTCL) is a primary cutaneous anaplastic large cell lymphoma (pcALCL). In some embodiments, the subject has received prior systemic treatment. In some embodiments, the non-Hodgkin lymphoma is anaplastic large cell lymphoma (ALCL). In some embodiments, the anaplastic large cell lymphoma (ALCL) is a systemic anaplastic large cell lymphoma (sALCL).

In some embodiments, the lymphoma is a Hodgkin lymphoma. In some embodiments, the subject has been previously treated for the Hodgkin lymphoma and the subject did not respond to treatment or relapsed after first-line treatment. In some embodiments, the subject relapsed after autologous stem cell transplant. In some embodiments, the subject relapsed after first-line treatment and the subject is ineligible for autologous stem cell transplant. In some embodiments, the subject has not been previously treated for the Hodgkin lymphoma. In some embodiments, the Hodgkin lymphoma is classical Hodgkin lymphoma (cHL). In some embodiments, the classical Hodgkin lymphoma (cHL) is advanced cHL. In some embodiments, the subject has been previously treated for cHL. In some embodiments, the subject has not been previously treated for cHL.

In some embodiments, the method further comprises administering one or more additional therapeutic agents capable of modulating the immune response. In some embodiments, the one or more additional therapeutic agents is not an antibody or antigen-binding fragment thereof. In some embodiments, the one or more additional therapeutic agents is an antibody or antigen-binding fragment thereof.

In some embodiments, the method further comprises administering one or more additional therapeutic agents. In some embodiments, the one or more additional therapeutic agents is a chemotherapy regimen consisting essentially of doxorubicin, vinblastine, and dacarbazine (AVD). In some embodiments, the one or more additional therapeutic agents is a chemotherapy regimen consisting essentially of Cyclophosphamide, Doxorubicin, and Prednisone (CHP). In some embodiments, the one or more additional therapeutic agents is an alkylating agent, an anthracycline, an antibiotic, an antifolate, an antimetabolite, an antitubulin agent, an auristatin, a chemotherapy sensitizer, a DNA minor groove binder, a DNA replication inhibitor, a duocarmycin, an etoposide, a fluorinated pyrimidine, a lexitropsin, a nitrosourea, a platinol, a purine antimetabolite, a puromycin, a radiation sensitizer, a steroid, a taxane, a topoisomerase inhibitor, and/or a vinca alkaloid. In some embodiments, the one or more additional therapeutic agents is selected from the group consisting of adriamycin, an androgen, anthramycin (AMC), asparaginase, 5-azacytidine, azathioprine, bleomycin, busulfan, buthionine sulfoximine, camptothecin, carboplatin, carmustine (BSNU), CC-1065, chlorambucil, cisplatin, colchicine, cyclophosphamide, cytarabine, cytidine arabinoside, cytochalasin B, dacarbazine, dactinomycin (formerly actinomycin), daunorubicin, decarbazine, docetaxel, doxorubicin, an estrogen, 5-fluordeoxyuridine, 5-fluorouracil, gramicidin D, hydroxydaunorubicin, hydroxyurea, idarubicin, ifosfamide, irinotecan, lomustine (CCNU), mechlorethamine, melphalan, 6-mercaptopurine, methotrexate, mithramycin, mitomycin C, mitoxantrone, nitroimidazole, paclitaxel, plicamycin, prednisone, prednisolone, procarbizine, streptozotocin, tenoposide, 6-thioguanine, thioTEPA, topotecan, vinblastine, vincristine, vinorelbine, VP-16 and VM-26. In some embodiments, the one or more additional therapeutic agents is an antibody or antigen-binding fragment thereof.

In some embodiments, the subject has cHL that has not been previously treated and the one or more additional therapeutic agents are adriamycin, dacarabazine and vinblastine (AVD). In some embodiments, the cHL is advanced cHL.

In some embodiments, the subject has a mature T-cell lymphoma that has not been previously treated and the one or more additional therapeutic agents are cyclophosphamide, hydroxydaunorubicin and prednisone (CHP). In some embodiments, the subject has cutaneous T-cell lymphoma (CTCL) and has been previously treated.

In some embodiments, the subject has a mature T-cell lymphoma that has not been previously treated and the one or more additional therapeutic agents are cyclophosphamide, hydroxydaunorubicin and prednisolone.

In some embodiments, the method further comprises treating the subject with irradiation.

In another aspect, the present invention provides for a method of increasing the ratio of CD8⁺ T cells to CD30⁺ T regulatory (Treg) cells in a subject having cancer comprising administering to the subject an antibody-drug conjugate, wherein the antibody-drug conjugate comprises an anti-CD30 antibody or an antigen-binding portion thereof conjugated to a monomethyl auristatin. In some embodiments, ratio of CD8⁺ T cells to CD30⁺ Treg cells is increased relative to the ratio of CD8⁺ T cells to CD30⁺ Treg cells in the subject prior to the administration of the antibody-drug conjugate.

In some embodiments, the CD30⁺ Treg cells are CD30⁺ inducible T regulatory (iTreg) cells or CD30⁺ peripheral T regulatory (pTreg) cells.

In some embodiments, the monomethyl auristatin is monomethyl auristatin E (MMAE). In some embodiments, the monomethyl auristatin is monomethyl auristatin F (MMAF).

In some embodiments, the anti-CD30 antibody is monoclonal anti-CD30 antibody AC10. In some embodiments, the anti-CD30 antibody is cAC10. In some embodiments, the antibody-drug conjugate is brentuximab vedotin.

In some embodiments, the anti-CD30 antibody comprises a heavy chain variable region and a light chain variable region, wherein the heavy chain variable region comprises:
(i) a CDR-H1 comprising the amino acid sequence of SEQ ID NO: 1;
(ii) a CDR-H2 comprising the amino acid sequence of SEQ ID NO: 2; and
(iii) a CDR-H3 comprising the amino acid sequence of SEQ ID NO: 3; and
   wherein the light chain variable region comprises:
(i) a CDR-L1 comprising the amino acid sequence of SEQ ID NO: 4;
(ii) a CDR-L2 comprising the amino acid sequence of SEQ ID NO: 5; and
(iii) a CDR-L3 comprising the amino acid sequence of SEQ ID NO: 6.

In some embodiments, the anti-CD30 antibody comprises a heavy chain variable region comprising the amino acid sequence of SEQ ID NO: 7 and a light chain variable region comprising the amino acid sequence of SEQ ID NO: 8.

In some embodiments, the antibody-drug conjugate comprises a linker between the anti-CD30 antibody or antigen-binding portion thereof and the monomethyl auristatin. In some embodiments, the linker is selected from the group consisting of a cleavable linker and a non-cleavable linker. In some embodiments, the linker is a cleavable peptide linker. In some embodiments, the linker is a protease-cleavable linker. In some embodiments, the protease cleavable linker is comprises a thiolreactive spacer and a dipeptide. In some embodiments, the protease cleavable linker comprises a thiolreactive maleimidocaproyl spacer, a valine-citrulline dipeptide, and a p-amino-benzyloxycarbonyl spacer. In some embodiments, the cleavable peptide linker has a formula: -MC-vc-PAB-. In some embodiments, the linker is a non-cleavable linker having a formula: -MC-.

In some embodiments, the subject has been previously treated for the cancer. In some embodiments, the subject did not respond to treatment or relapsed after first-line treatment. In some embodiments, the subject has not previously been treated for the cancer.

In some embodiments, the cancer is a lymphoma. In some embodiments, the lymphoma is a T-cell lymphoma. In some embodiments, the lymphoma is a B-cell lymphoma.

In some embodiments, the lymphoma is a non-Hodgkin lymphoma. In some embodiments, the subject has been previously treated for the non-Hodgkin lymphoma and the subject did not respond to treatment or relapsed after first-line treatment. In some embodiments, the subject has not been previously treated for the non-Hodgkin lymphoma. In some embodiments, the non-Hodgkin lymphoma is a mature T-cell lymphoma. In some embodiments, the non-Hodgkin lymphoma is diffuse large B-cell lymphoma (DLBCL), peripheral T-cell lymphoma (PTCL), anaplastic large cell lymphoma (ALCL) or cutaneous T-cell lymphoma (CTCL). In some embodiments, the non-Hodgkin lymphoma is cutaneous T-cell lymphoma (CTCL). In some embodiments, the cutaneous T-cell lymphoma (CTCL) is a mycosis fungoides (MF). In some embodiments, the mycosis fungoides is a CD30-positive mycosis fungoides (MF). In some embodiments, the cutaneous T-cell lymphoma (CTCL) is a primary cutaneous anaplastic large cell lymphoma (pcALCL). In some embodiments, the subject has received prior systemic treatment. In some embodiments, the non-Hodgkin lymphoma is anaplastic large cell lymphoma (ALCL). In some embodiments, the anaplastic large cell lymphoma (ALCL) is a systemic anaplastic large cell lymphoma (sALCL).

In some embodiments, the lymphoma is a Hodgkin lymphoma. In some embodiments, the subject has been previously treated for the Hodgkin lymphoma and the subject did not respond to treatment or relapsed after first-line treatment. In some embodiments, the subject relapsed after autologous stem cell transplant. In some embodiments, the subject relapsed after first-line treatment and the subject is ineligible for autologous stem cell transplant. In some embodiments, the subject has not been previously treated for the Hodgkin lymphoma. In some embodiments, the Hodgkin lymphoma is classical Hodgkin lymphoma (cHL). In some embodiments, the classical Hodgkin lymphoma (cHL) is advanced cHL. In some embodiments, the subject has been previously treated for cHL. In some embodiments, the subject has not been previously treated for cHL.

In some embodiments, the method further comprises administering one or more additional therapeutic agents capable of modulating the immune response. In some embodiments, the one or more additional therapeutic agents is not an antibody or antigen-binding fragment thereof. In some embodiments, the one or more additional therapeutic agents is an antibody or antigen-binding fragment thereof.

In some embodiments, the method further comprises administering one or more additional therapeutic agents. In some embodiments, the one or more additional therapeutic agents is a chemotherapy regimen consisting essentially of doxorubicin, vinblastine, and dacarbazine (AVD). In some embodiments, the one or more additional therapeutic agents is a chemotherapy regimen consisting essentially of Cyclophosphamide, Doxorubicin, and Prednisone (CHP). In some embodiments, the one or more additional therapeutic agents is an alkylating agent, an anthracycline, an antibiotic, an antifolate, an antimetabolite, an antitubulin agent, an auristatin, a chemotherapy sensitizer, a DNA minor groove binder, a DNA replication inhibitor, a duocarmycin, an etoposide, a fluorinated pyrimidine, a lexitropsin, a nitrosourea, a platinol, a purine antimetabolite, a puromycin, a radiation sensitizer, a steroid, a taxane, a topoisomerase inhibitor, and/or a vinca alkaloid. In some embodiments, the one or more additional therapeutic agents is selected from the group consisting of adriamycin, an androgen, anthramycin (AMC), asparaginase, 5-azacytidine, azathioprine, bleomycin, busulfan, buthionine sulfoximine, camptothecin, carboplatin, carmustine (BSNU), CC-1065, chlorambucil, cisplatin, colchicine, cyclophosphamide, cytarabine, cytidine arabinoside, cytochalasin B, dacarbazine, dactinomycin (formerly actinomycin), daunorubicin, decarbazine, docetaxel, doxorubicin, an estrogen, 5-fluordeoxyuridine, 5-fluorouracil, gramicidin D, hydroxydaunorubicin, hydroxyurea, idarubicin, ifosfamide, irinotecan, lomustine (CCNU), mechlorethamine, melphalan, 6-mercaptopurine, methotrexate, mithramycin, mitomycin C, mitoxantrone, nitroimidazole, paclitaxel, plicamycin, prednisone, prednisolone, procarbizine, streptozotocin, tenoposide, 6-thioguanine, thioTEPA, topotecan, vinblastine, vincristine, vinorelbine, VP-16 and VM-26. In some embodiments, the one or more additional therapeutic agents is an antibody or antigen-binding fragment thereof.

In some embodiments, the subject has cHL that has not been previously treated and the one or more additional therapeutic agents are adriamycin, dacarabazine and vinblastine. In some embodiments, the cHL is advanced cHL.

In some embodiments, the subject has a mature T-cell lymphoma that has not been previously treated and the one or more additional therapeutic agents are cyclophosphamide, hydroxydaunorubicin and prednisone.

In some embodiments, the subject has a mature T-cell lymphoma that has not been previously treated and the one or more additional therapeutic agents are cyclophosphamide, hydroxydaunorubicin and prednisolone.

In some embodiments, the method further comprises treating the subject with irradiation.

In another aspect, the present invention provides for a method of modulating the immune response in a subject having cancer comprising administering to the subject an antibody-drug conjugate, wherein the antibody-drug conjugate comprises an anti-CD30 antibody or an antigen-binding portion thereof conjugated to a monomethyl auristatin, wherein the modulation comprises increasing the ratio of CD8⁺ T cells to CD30⁺ T regulatory (Treg) cells in the subject. In some embodiments, ratio of CD8⁺ T cells to CD30⁺ Treg cells is increased relative to the ratio of CD8⁺ T cells to CD30⁺ Treg cells in the subject prior to the administration of the antibody-drug conjugate.

In some embodiments, the CD30⁺ Treg cells are CD30⁺ inducible T regulatory (iTreg) cells or CD30⁺ peripheral T regulatory (pTreg) cells.

In some embodiments, the monomethyl auristatin is monomethyl auristatin E (MMAE). In some embodiments, the monomethyl auristatin is monomethyl auristatin F (MMAF).

In some embodiments, the anti-CD30 antibody is monoclonal anti-CD30 antibody AC10. In some embodiments, the anti-CD30 antibody is cAC10. In some embodiments, the antibody-drug conjugate is brentuximab vedotin.

In some embodiments, the anti-CD30 antibody comprises a heavy chain variable region and a light chain variable region, wherein the heavy chain variable region comprises:
(i) a CDR-H1 comprising the amino acid sequence of SEQ ID NO: 1;
(ii) a CDR-H2 comprising the amino acid sequence of SEQ ID NO: 2; and
(iii) a CDR-H3 comprising the amino acid sequence of SEQ ID NO: 3; and
   wherein the light chain variable region comprises:
(i) a CDR-L1 comprising the amino acid sequence of SEQ ID NO: 4;
(ii) a CDR-L2 comprising the amino acid sequence of SEQ ID NO: 5; and
(iii) a CDR-L3 comprising the amino acid sequence of SEQ ID NO: 6.

In some embodiments, the anti-CD30 antibody comprises a heavy chain variable region comprising the amino acid sequence of SEQ ID NO: 7 and a light chain variable region comprising the amino acid sequence of SEQ ID NO: 8.

In some embodiments, the antibody-drug conjugate comprises a linker between the anti-CD30 antibody or antigen-binding portion thereof and the monomethyl auristatin. In some embodiments, the linker is selected from the group consisting of a cleavable linker and a non-cleavable linker. In some embodiments, the linker is a cleavable peptide linker. In some embodiments, the linker is a protease-cleavable linker. In some embodiments, the protease cleavable linker is comprises a thiolreactive spacer and a dipeptide. In some embodiments, the protease cleavable linker comprises a thiolreactive maleimidocaproyl spacer, a valine-citrulline dipeptide, and a p-amino-benzyloxycarbonyl spacer. In some embodiments, the cleavable peptide linker has a formula: -MC-vc-PAB-. In some embodiments, the linker is a non-cleavable linker having a formula: -MC-.

In some embodiments, the subject has been previously treated for the cancer. In some embodiments, the subject did not respond to treatment or relapsed after first-line treatment. In some embodiments, the subject has not previously been treated for the cancer.

In some embodiments, the cancer is a lymphoma. In some embodiments, the lymphoma is a T-cell lymphoma. In some embodiments, the lymphoma is a B-cell lymphoma.

In some embodiments, the lymphoma is a non-Hodgkin lymphoma. In some embodiments, the subject has been previously treated for the non-Hodgkin lymphoma and the subject did not respond to treatment or relapsed after first-line treatment. In some embodiments, the subject has not been previously treated for the non-Hodgkin lymphoma. In some embodiments, the non-Hodgkin lymphoma is a mature T-cell lymphoma. In some embodiments, the non-Hodgkin lymphoma is diffuse large B-cell lymphoma (DLBCL), peripheral T-cell lymphoma (PTCL), anaplastic large cell lymphoma (ALCL) or cutaneous T-cell lymphoma (CTCL). In some embodiments, the non-Hodgkin lymphoma is cutaneous T-cell lymphoma (CTCL). In some embodiments, the cutaneous T-cell lymphoma (CTCL) is a mycosis fungoides (MF). In some embodiments, the mycosis fungoides is a CD30-positive mycosis fungoides (MF). In some embodiments, the cutaneous T-cell lymphoma (CTCL) is a primary cutaneous anaplastic large cell lymphoma (pcALCL). In some embodiments, the subject has received prior systemic treatment. In some embodiments, the non-Hodgkin lymphoma is anaplastic large cell lymphoma (ALCL). In some embodiments, the anaplastic large cell lymphoma (ALCL) is a systemic anaplastic large cell lymphoma (sALCL).

In some embodiments, the lymphoma is a Hodgkin lymphoma. In some embodiments, the subject has been previously treated for the Hodgkin lymphoma and the subject did not respond to treatment or relapsed after first-line treatment. In some embodiments, the subject relapsed after autologous stem cell transplant. In some embodiments, the subject relapsed after first-line treatment and the subject is ineligible for autologous stem cell transplant. In some embodiments, the subject has not been previously treated for the Hodgkin lymphoma. In some embodiments, the Hodgkin lymphoma is classical Hodgkin lymphoma (cHL). In some embodiments, the classical Hodgkin lymphoma (cHL) is advanced cHL. In some embodiments, the subject has been previously treated for cHL. In some embodiments, the subject has not been previously treated for cHL.

In some embodiments, the method further comprises administering one or more additional therapeutic agents capable of modulating the immune response. In some embodiments, the one or more additional therapeutic agents is not an antibody or antigen-binding fragment thereof. In some embodiments, the one or more additional therapeutic agents is an antibody or antigen-binding fragment thereof.

In some embodiments, the method further comprises administering one or more additional therapeutic agents. In some embodiments, the one or more additional therapeutic agents is a chemotherapy regimen consisting essentially of doxorubicin, vinblastine, and dacarbazine (AVD). In some embodiments, the one or more additional therapeutic agents is a chemotherapy regimen consisting essentially of Cyclophosphamide, Doxorubicin, and Prednisone (CHP). In some embodiments, the one or more additional therapeutic agents is an alkylating agent, an anthracycline, an antibiotic, an antifolate, an antimetabolite, an antitubulin agent, an auristatin, a chemotherapy sensitizer, a DNA minor groove binder, a DNA replication inhibitor, a duocarmycin, an etoposide, a fluorinated pyrimidine, a lexitropsin, a nitrosourea, a platinol, a purine antimetabolite, a puromycin, a radiation sensitizer, a steroid, a taxane, a topoisomerase inhibitor, and/or a vinca alkaloid. In some embodiments, the one or more additional therapeutic agents is selected from the group consisting of adriamycin, an androgen, anthramycin (AMC), asparaginase, 5-azacytidine, azathioprine, bleomycin, busulfan, buthionine sulfoximine, camptothecin, carboplatin, carmustine (BSNU), CC-1065, chlorambucil, cisplatin, colchicine, cyclophosphamide, cytarabine, cytidine arabinoside, cytochalasin B, dacarbazine, dactinomycin (formerly actinomycin), daunorubicin, decarbazine, docetaxel, doxorubicin, an estrogen, 5-fluordeoxyuridine, 5-fluorouracil, gramicidin D, hydroxydaunorubicin, hydroxyurea, idarubicin, ifosfamide, irinotecan, lomustine (CCNU), mechlorethamine, melphalan, 6-mercaptopurine, methotrexate, mithramycin, mitomycin C, mitoxantrone, nitroimidazole, paclitaxel, plicamycin, prednisone, prednisolone, procarbizine, streptozotocin, tenoposide, 6-thioguanine, thioTEPA, topotecan, vinblastine, vincristine, vinorelbine, VP-16 and VM-26. In some embodiments, the one or more additional therapeutic agents is an antibody or antigen-binding fragment thereof.

In some embodiments, the subject has cHL that has not been previously treated and the one or more additional therapeutic agents are adriamycin, dacarabazine and vinblastine. In some embodiments, the cHL is advanced cHL.

In some embodiments, the subject has a mature T-cell lymphoma that has not been previously treated and the one or more additional therapeutic agents are cyclophosphamide, hydroxydaunorubicin and prednisone.

In some embodiments, the subject has a mature T-cell lymphoma that has not been previously treated and the one or more additional therapeutic agents are cyclophosphamide, hydroxydaunorubicin and prednisolone.

In some embodiments, the method further comprises treating the subject with irradiation.

It is to be understood that one, some, or all of the properties of the various embodiments described herein may be combined to form other embodiments of the present invention. These and other aspects of the invention will become apparent to one of skill in the art. These and other embodiments of the invention are further described by the detailed description that follows.

### BRIEF DESCRIPTION OF THE DRAWINGS

FIGS. 1A and 1B is a series of graphs showing that brentuximab vedotin (BV) impaired T regulatory cells in vitro. A) BV drove a dose-dependent reduction in total viable iTreg numbers from five separate donors. B) BV showed enhanced depletion of CD30⁺iTregs. Cell counts are shown as the percent of untreated control. ADC indicates antibody drug-conjugate. IgG MMAE indicates control ADC.

FIG. 2A-D is a series of graphs showing that treatment with BV reduced the number of inducible T regulatory cells while relieving the repression of in vitro CD8⁺ T cell proliferation. Increasing the iTreg to CD8⁺ T cell ratio (iTreg:CD8 ratio) of cells isolated from A) Donor 1 or B) Donor 2 abrogated T cell expansion. Increasing concentrations of BV treatment selectively reduced iTregs and augmented CD8⁺ T cell accumulation for both C) Donor 1 and D) Donor 2. Cell counts are shown as the percent of untreated control. ADC indicates antibody drug-conjugate. IgG MMAE indicates control ADC.

FIGS. 3A and 3B is a series of graphs showing that BV depleted naturally occurring CD30⁺ blood Tregs but not CD30⁺ CD8⁺ T cells in vitro. A) BV drove a dose-dependent reduction of viable CD30⁺ Treg numbers from four separate donors. B) BV did not deplete CD30⁺ CD8⁺ T cells. ADC indicates antibody drug-conjugate. IgG MMAE indicates control ADC.

FIG. 4A-C is a series of graphs showing that BV reduced T regulatory cells and increased the CD8⁺ T cell to Treg ratio in a xeno-GVHD mouse model. A) BV significantly reduced human T regulatory cells in the spleen compared to PBS alone (untreated). B) Splenic CD8⁺ T cells were unaffected by BV treatment with a trend toward increased numbers. C) BV treatment increased the CD8⁺ T cell/Treg ratio in vivo.

FIG. 5 is a graph showing that single treatment with BV in patients with classical Hodgkin lymphoma resulted in the reduction of T helper cells subset populations.

FIG. 6 is a graph showing the expression of CD30 in T cell subtypes isolated from human blood.

FIG. 7 is a graph showing that single treatment with BV in patients with classical Hodgkin lymphoma reduced the number of T regulatory cells that expressed CD30 (CD30+) as compared to the number of T regulatory cells that did not express CD30 (CD30−). BSLN indicates baseline measurement.

FIGS. 8A and 8B is a series of graphs showing that CD30 expression is enriched on CD25^{hi} CCR4^{hi} FoxP3^{hi} effector Tregs in PBMC. A) CD30 is most frequently expressed by T regulatory cells compared to CD4⁺ and CD8⁺ memory and naïve T cell subsets. B) Expression of CD30 is highly associated with the effector T regulatory subset (FoxP3^{hi} CD25^{hi} CCR4^{hi}).

FIG. 9A-D is a series of graphs showing that activated T regulatory cells demonstrate heightened CD30 receptor expression and payload delivery, along with impaired drug efflux capacity. A) A higher proportion of enriched T regulatory cells express CD30 compared to CD4⁺ and CD8⁺ T cells following activation. B) Enriched T regulatory cells have an increased magnitude of expression of CD30 by Mean Fluorescence Intensity (MFI) compared to CD4⁺ and CD8⁺ T cells following activation. C) T regulatory cells show accelerated and increased release of fluorescent payload from a conditionally fluorescent anti-CD30 mAb relative to CD4⁺ and CD8⁺ T cells in an internalization assay. D) T regulatory cells show the slowest rhodamine-123 efflux among T cell subsets while CD8⁺ T cells show rapid clearance of intracellular rhodamine-123 in a rhodamine 123 efflux assay.

### DETAILED DESCRIPTION

### I. Definitions

In order that the present disclosure can be more readily understood, certain terms are first defined. As used in this application, except as otherwise expressly provided herein, each of the following terms shall have the meaning set forth below. Additional definitions are set forth throughout the application.

The term “and/or” where used herein is to be taken as specific disclosure of each of the two specified features or components with or without the other. Thus, the term “and/or” as used in a phrase such as “A and/or B” herein is intended to include “A and B,” “A or B,” “A” (alone), and “B” (alone). Likewise, the term “and/or” as used in a phrase such as “A, B, and/or C” is intended to encompass each of the following aspects: A, B, and C; A, B, or C; A or C; A or B; B or C; A and C; A and B; B and C; A (alone); B (alone); and C (alone).

The term “about” as used herein refers to the usual error range for the respective value readily known to the skilled person in this technical field. Reference to “about” a value or parameter herein includes (and describes) embodiments that are directed to that value or parameter per se.

It is understood that aspects and embodiments of the invention described herein include “comprising,” “consisting,” and “consisting essentially of” aspects and embodiments.

Unless defined otherwise, all technical and scientific terms used herein have the same meaning as commonly understood by one of ordinary skill in the art to which this disclosure is related. For example, the Concise Dictionary of Biomedicine and Molecular Biology, Juo, Pei-Show, 2nd ed., 2002, CRC Press; The Dictionary of Cell and Molecular Biology, 3rd ed., 1999, Academic Press; and the Oxford Dictionary Of Biochemistry And Molecular Biology, Revised, 2000, Oxford University Press, provide one of skill with a general dictionary of many of the terms used in this disclosure.

Units, prefixes, and symbols are denoted in their Systeme International de Unites (SI) accepted form. Numeric ranges are inclusive of the numbers defining the range. The headings provided herein are not limitations of the various aspects of the disclosure, which can be had by reference to the specification as a whole. Accordingly, the terms defined immediately below are more fully defined by reference to the specification in its entirety.

“Administering” refers to the physical introduction of a therapeutic agent to a subject, using any of the various methods and delivery systems known to those skilled in the art. Exemplary routes of administration include intravenous, intramuscular, subcutaneous, intraperitoneal, spinal or other parenteral routes of administration, for example by injection or infusion. The phrase “parenteral administration” as used herein means modes of administration other than enteral and topical administration, usually by injection, and includes, without limitation, intravenous, intramuscular, intraarterial, intrathecal, intralymphatic, intralesional, intracapsular, intraorbital, intracardiac, intradermal, intraperitoneal, transtracheal, subcutaneous, subcuticular, intraarticular, subcapsular, subarachnoid, intraspinal, epidural and intrasternal injection and infusion, as well as in vivo electroporation. A therapeutic agent can be administered via a non-parenteral route, or orally. Other non-parenteral routes include a topical, epidermal or mucosal route of administration, for example, intranasally, vaginally, rectally, sublingually or topically. Administering can also be performed, for example, once, a plurality of times, and/or over one or more extended periods.

An “adverse event” (AE) as used herein is any unfavorable and generally unintended or undesirable sign (including an abnormal laboratory finding), symptom, or disease associated with the use of a medical treatment. A medical treatment can have one or more associated AEs and each AE can have the same or different level of severity. Reference to methods capable of “altering adverse events” means a treatment regime that decreases the incidence and/or severity of one or more AEs associated with the use of a different treatment regime.

An “antibody” (Ab) shall include, without limitation, a glycoprotein immunoglobulin which binds specifically to an antigen and comprises at least two heavy (H) chains and two light (L) chains interconnected by disulfide bonds, or an antigen-binding portion thereof. Each H chain comprises a heavy chain variable region (abbreviated herein as V_{H}) and a heavy chain constant region. The heavy chain constant region comprises at least three constant domains, C_{H1}, C_{H2} and C_{H3}. Each light chain comprises a light chain variable region (abbreviated herein as V_{L}) and a light chain constant region. The light chain constant region comprises one constant domain, C_{L}. The V_{H} and V_{L} regions can be further subdivided into regions of hypervariability, termed complementarity determining regions (CDRs), interspersed with regions that are more conserved, termed framework regions (FRs). Each V_{H} and V_{L} comprises three CDRs and four FRs, arranged from amino-terminus to carboxy-terminus in the following order: FR1, CDR1, FR2, CDR2, FR3, CDR3, and FR4. The variable regions of the heavy and light chains contain a binding domain that interacts with an antigen. The constant regions of the antibodies can mediate the binding of the immunoglobulin to host tissues or factors, including various cells of the immune system (e.g., effector cells) and the first component (C1q) of the classical complement system.

An immunoglobulin can derive from any of the commonly known isotypes, including but not limited to IgA, secretory IgA, IgG, and IgM. IgG subclasses are also well known to those in the art and include but are not limited to human IgG1, IgG2, IgG3 and IgG4. “Isotype” refers to the antibody class or subclass (e.g., IgM or IgG1) that is encoded by the heavy chain constant region genes. The term “antibody” includes, by way of example, both naturally occurring and non-naturally occurring antibodies; monoclonal and polyclonal antibodies; chimeric and humanized antibodies; human or non-human antibodies; wholly synthetic antibodies; and single chain antibodies. A non-human antibody can be humanized by recombinant methods to reduce its immunogenicity in man. Where not expressly stated, and unless the context indicates otherwise, the term “antibody” also includes an antigen-binding fragment or an antigen-binding portion of any of the aforementioned immunoglobulins, and includes a monovalent and a divalent fragment or portion, and a single chain antibody.

An “isolated antibody” refers to an antibody that is substantially free of other antibodies having different antigenic specificities (e.g., an isolated antibody that binds specifically to CD30 is substantially free of antibodies that bind specifically to antigens other than CD30). An isolated antibody that binds specifically to CD30 can, however, have cross-reactivity to other antigens, such as CD30 molecules from different species. Moreover, an isolated antibody can be substantially free of other cellular material and/or chemicals. In one embodiment, an antibody includes a conjugate attached to another agent (e.g., small molecule drug). In some embodiments, an anti-CD30 antibody includes a conjugate of an anti-CD30 antibody with a small molecule drug (e.g., MMAE or MMAF).

The term “monoclonal antibody” (mAb) refers to a non-naturally occurring preparation of antibody molecules of single molecular composition, i.e., antibody molecules whose primary sequences are essentially identical, and which exhibits a single binding specificity and affinity for a particular epitope. A monoclonal antibody is an example of an isolated antibody. Monoclonal antibodies can be produced by hybridoma, recombinant, transgenic, or other techniques known to those skilled in the art.

A “human antibody” (HuMAb) refers to an antibody having variable regions in which both the FRs and CDRs are derived from human germline immunoglobulin sequences. Furthermore, if the antibody contains a constant region, the constant region also is derived from human germline immunoglobulin sequences. The human antibodies of the disclosure can include amino acid residues not encoded by human germline immunoglobulin sequences (e.g., mutations introduced by random or site-specific mutagenesis in vitro or by somatic mutation in vivo). However, the term “human antibody,” as used herein, is not intended to include antibodies in which CDR sequences derived from the germline of another mammalian species, such as a mouse, have been grafted onto human framework sequences. The terms “human antibodies” and “fully human antibodies” and are used synonymously.

A “humanized antibody” refers to an antibody in which some, most, or all of the amino acids outside the CDRs of a non-human antibody are replaced with corresponding amino acids derived from human immunoglobulins. In one embodiment of a humanized form of an antibody, some, most, or all of the amino acids outside the CDRs have been replaced with amino acids from human immunoglobulins, whereas some, most, or all amino acids within one or more CDRs are unchanged. Small additions, deletions, insertions, substitutions or modifications of amino acids are permissible as long as they do not abrogate the ability of the antibody to bind to a particular antigen. A “humanized antibody” retains an antigenic specificity similar to that of the original antibody. In some embodiments, the CDRs of a humanized antibody contain CDRs from a non-human, mammalian antibody. In other embodiments, the CDRs of a humanized antibody contain CDRs from an engineered, synthetic antibody.

A “chimeric antibody” refers to an antibody in which the variable regions are derived from one species and the constant regions are derived from another species, such as an antibody in which the variable regions are derived from a mouse antibody and the constant regions are derived from a human antibody.

An “anti-antigen antibody” refers to an antibody that binds specifically to the antigen. For example, an anti-CD30 antibody binds specifically to CD30.

An “antigen-binding portion” of an antibody (also called an “antigen-binding fragment”) refers to one or more fragments of an antibody that retain the ability to bind specifically to the antigen bound by the whole antibody. Examples of antibody fragments include but are not limited to Fv, Fab, Fab′, Fab′-SH, F(ab′)₂; diabodies; linear antibodies; single-chain antibody molecules (e.g. scFv); and multispecific antibodies formed from antibody fragments. Papain digestion of antibodies produces two identical antigen-binding fragments, called “Fab” fragments, each with a single antigen-binding site, and a residual “Fc” fragment, whose name reflects its ability to crystallize readily. Pepsin treatment yields an F(ab′)₂ fragment that has two antigen-combining sites and is still capable of cross-linking antigen.

The term “variable” refers to the fact that certain segments of the variable domains differ extensively in sequence among antibodies. The V domain mediates antigen binding and defines the specificity of a particular antibody for its particular antigen. However, the variability is not evenly distributed across the entire span of the variable domains. Instead, it is concentrated in three segments called complementarity determining regions (CDRs) both in the light-chain and the heavy chain variable domains. The more highly conserved portions of variable domains are called the framework regions (FR). The variable domains of native heavy and light chains each comprise four FR regions, largely adopting a beta-sheet configuration, connected by three CDRs, which form loops connecting, and in some cases forming part of, the beta-sheet structure. The CDRs in each chain are held together in close proximity by the FR regions and, with the CDRs from the other chain, contribute to the formation of the antigen binding site of antibodies (see Kabat et al, Sequences of Immunological Interest, Fifth Edition, National Institute of Health, Bethesda, Md. (1991)). The constant domains are not involved directly in the binding of antibody to an antigen, but exhibit various effector functions, such as participation of the antibody in antibody-dependent cellular toxicity.

The “variable region” or “variable domain” of an antibody refers to the arnino-terminal domains of the heavy or light chain of the antibody. The variable domains of the heavy chain and light chain may be referred to as “VH’ and “VL”, respectively. These domains are generally the most variable parts of the antibody (relative to other antibodies of the same class) and contain the antigen binding sites.

The term “hypervariable region,” “HVR,” or “HV,” when used herein refers to the regions of an antibody-variable domain that are hypervariable in sequence and/or form structurally defined loops. Generally, antibodies comprise six HVRs; three in the VH (H1, H2, H3), and three in the VL (L1, L2, L3). In native antibodies, H3 and L3 display the most diversity of the six HVRs, and H3 in particular is believed to play a unique role in conferring fine specificity to antibodies. See, e.g., Xu et al. Immunity 13:37-45 (2000); Johnson and Wu in Methods in Molecular Biology 248:1-25 (Lo, ed., Human Press, Totowa, N.J., 2003)). Indeed, naturally occurring camelid antibodies consisting of a heavy chain only are functional and stable in the absence of light chain. See, e.g., Hamers-Casterman et al., Nature 363:446-448 (1993) and Sheriff et al., Nature Struct. Biol. 3:733-736 (1996).

A number of HVR delineations are in use and are encompassed herein. The HVRs that are Kabat complementarity-determining regions (CDRs) are based on sequence variability and are the most commonly used (Kabat et al., Sequences of Proteins of Immunological Interest, 5^{th} Ed. Public Health Service, National Institute of Health, Bethesda, Md. (1991)). Chothia HVRs refer instead to the location of the structural loops (Chothia and Lesk J. Mol. Biol. 196:901-917 (1987)). The “contact” HVRs are based on an analysis of the available complex crystal structures. The residues from each of these HVRs are noted below.

| Loop | Kabat | Chothia | Contact |
|---|---|---|---|
| L1 | L24-L34 | L26-L34 | L30-L36 |
| L2 | L50-L56 | L50-L56 | L46-L55 |
| L3 | L89-L97 | L91-L96 | L89-L96 |
| H1 | H31-H35B | H26-H32 | H30-H35B (Kabat Numbering) |
| H1 | H31-H35 | H26-H32 | H30-H35 (Chothia Numbering) |
| H2 | H50-H65 | H53-H56 | H47-H58 |
| H3 | H95-H102 | H95-H102 | H93-H101 |

Unless otherwise indicated, the variable-domain residues (HVR residues and framework region residues) are numbered according to Kabat et al., supra.

“Framework” or “FR” residues are those variable-domain residues other than the HVR residues as herein defined.

The expression “variable-domain residue-numbering as in Kabat” or “amino-acid-position numbering as in Kabat,” and variations thereof, refers to the numbering system used for heavy-chain variable domains or light-chain variable domains of the compilation of antibodies in Kabat et al., supra. Using this numbering system, the actual linear amino acid sequence may contain fewer or additional amino acids corresponding to a shortening of, or insertion into, a FR or HVR of the variable domain. For example, a heavy-chain variable domain may include a single amino acid insert (residue 52a according to Kabat) after residue 52 of H2 and inserted residues (e.g. residues 82a, 82b, and 82c, etc. according to Kabat) after heavy-chain FR residue 82. The Kabat numbering of residues may be determined for a given antibody by alignment at regions of homology of the sequence of the antibody with a “standard” Kabat numbered sequence.

As used herein, the term “specifically binds to” or is “specific for” refers to measurable and reproducible interactions such as binding between a target and an antibody, which is determinative of the presence of the target in the presence of a heterogeneous population of molecules including biological molecules. For example, an antibody that specifically binds to a target (which can be an epitope) is an antibody that binds this target with greater affinity, avidity, more readily, and/or with greater duration than it binds to other targets. In one embodiment, the extent of binding of an antibody to an unrelated target is less than about 10% of the binding of the antibody to the target as measured, e.g., by a radioimmunoassay (RIA). In certain embodiments, an antibody that specifically binds to a target has a dissociation constant (Kd) of <I μM, <100 nM, <10 nM, <1 nM, or <0.1 nM. In certain embodiments, an antibody specifically binds to an epitope on a protein that is conserved among the protein from different species. In another embodiment, specific binding can include, but does not require exclusive binding.

The abbreviations “vc” and “val-cit” refer to the dipeptide valine-citrulline.

The abbreviation “PAB” refers to the self-immolative spacer:

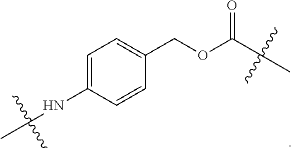 [SMILES: *Nc1ccc(COC(*)=O)cc1]

The abbreviation “MC” refers to the stretcher maleimidocaproyl:

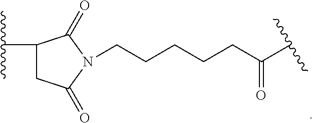 [SMILES: *C(=O)CCCCCN1C(=O)CC(*)C1=O]

The term “cAC10-MC-vc-PAB-MMAE” refers to a chimeric AC10 antibody conjugated to the drug MMAE through a MC-vc-PAB linker.

An “anti-CD30 vc-PAB-MMAE antibody-drug conjugate” refers to an anti-CD30 antibody conjugated to the drug MMAE via a linker comprising the dipeptide valine citrulline and the self-immolative spacer PAB as shown in Formula (I) of U.S. Pat. No. 9,211,319.

A “cancer” refers a broad group of various diseases characterized by the uncontrolled growth of abnormal cells in the body. A “cancer” or “cancer tissue” can include a tumor. Unregulated cell division and growth results in the formation of malignant tumors that invade neighboring tissues and can also metastasize to distant parts of the body through the lymphatic system or bloodstream. Following metastasis, the distal tumors can be said to be “derived from” the pre-metastasis tumor. For example, a “tumor derived from” a non-Hodgkin lymphoma refers to a tumor that is the result of a metastasized non-Hodgkin lymphoma. Because the distal tumor is derived from the pre-metastasis tumor, the “derived from” tumor can also comprise the pre-metastasis tumor, e.g., a tumor derived from a non-Hodgkin lymphoma can comprise a non-Hodgkin lymphoma.

“CD30” or “TNFRSF8” refers to a receptor that is a member of the tumor necrosis factor receptor superfamily. CD30 is a transmembrane glycoprotein expressed on activated CD4⁺ and CD8⁺ T cells and B cells, and virally-infected lymphocytes. CD30 interacts with TRAF2 and TRAF3 to mediate signal transduction that leads to activation of NF-κB. CD30 acts as a positive regulator of apoptosis, and it has been shown to limit the proliferative potential of auto-reactive CD8 effector T cells. CD30 is also expressed by various forms of lymphoma, including Hodgkin lymphoma (CD30 is expressed by Reed-Sternberg cells) and non-Hodgkin lymphoma (e.g., diffuse large B-cell lymphoma (DLBCL), peripheral T-cell lymphoma (PTCL), and cutaneous T-cell lymphoma (CTCL).

The terms “Treg” or “regulatory T cell” refer to CD4⁺ T cells that suppresses CD4 CD25⁺ and CD8⁺ T cell proliferation and/or effector function, or that otherwise down-modulate an immune response. Notably, Treg may down-regulate immune responses mediated by Natural Killer cells, Natural Killer T cells as well as other immune cells.

The terms “regulatory T cell function” or “a function of Treg” are used interchangeably to refer to any biological function of a Treg that results in a reduction in CD4 CD25⁺ or CD8⁺ T cell proliferation or a reduction in an effector T cell-mediated immune response. Treg function can be measured via techniques established in the art. Non-limiting examples of useful in vitro assays for measuring Treg function include Transwell suppression assays as well as in vitro assays in which the target conventional T cells (Tconv) and Tregs purified from human peripheral blood or umbilical cord blood (or murine spleens or lymph nodes) are optionally activated by anti-CD3⁺ anti-CD28 coated beads (or antigen-presenting cells (APCs) such as, e.g., irradiated splenocytes or purified dendritic cells (DCs) or irradiated PBMCs) followed by in vitro detection of conventional T cell proliferation (e.g., by measuring incorporation of radioactive nucleotides (such as, e.g., [H]-thynidine) or fluorescent nucleotides, or by Cayman Chemical MTT Cell Proliferation Assay Kit, or by monitoring the dilution of a green fluorochrome ester CFSE or Seminaphtharhodafluor (SNARF-1) dye by flow cytometry). Other common assays measure T cell cytokine responses. Useful in vivo assays of Treg function include assays in animal models of diseases in which Tregs play an important role, including, e.g., (1) homeostasis model (using naïve homeostatically expanding CD4⁺ T cells as target cells that are primarily suppressed by Tregs), (2) inflammatory bowel disease (IBD) recovery model (using Th1 T cells (Th17) as target cells that are primarily suppressed by Tregs), (3) experimental autoimmune encephalomyelitis (EAE) model (using Th17 and Th1 T cells as target cells that are primarily suppressed by Tregs), (4) B16 melanoma model (suppression of antitumor immunity) (using CD8⁺ T cells as target cells that are primarily suppressed by Tregs), (5) suppression of colon inflammation in adoptive transfer colitis where naïve CD4⁺ CD45RB^{M} Tconv cells are transferred into RagV mice, and (6) Foxp3 rescue model (using lymphocytes as target cells that are primarily suppressed by Tregs). According to one protocol, all of the models require mice for donor T cell populations as well as Ragl^{-/-} or Foxp3 mice for recipients. For more details on various useful assays see, e.g., Collison and Vignali, In Vitro Treg Suppression Assays, Chapter 2 in Regulatory T Cells: Methods and Protocols, Methods in Molecular Biology, Kassiotis and Liston eds., Springer, 2011, 707:21-37; Workman et al, In Vivo Treg Suppression Assays, Chapter 9 in Regulatory T Cells: Methods and Protocols, Methods in Molecular Biology, Kassiotis and Liston eds., Springer, 2011, 119-156; Takahashi et al, Int. Immunol, 1998, 10: 1969-1980; Thornton et al, J. Exp. Med., 1998, 188:287-296; Collison et al, J. Immunol, 2009, 182:6121-6128; Thornton and Shevach. J. Exp. Med., 1998, 188:287-296; Assenan et al, J. Exp. Med., 1999, 190:995-1004; Dieckmann et al, J. Exp. Med., 2001, 193: 1303-1310; Belkaid, Nature Reviews, 2007, 7:875-888; Tang and Bluestone, Nature Immunology, 2008, 9:239-244; Bettini and Vignali, Curr. Opin. Immunol, 2009, 21:612-618; Dannull et al, J Clin Invest. 2005, 115(12):3623-33; Tsaknaridis, et al, J Neurosci Res., 2003, 74:296-308.

The term “immunotherapy” refers to the treatment of a subject afflicted with, at risk of contracting, or suffering a recurrence of a disease by a method comprising inducing, enhancing, suppressing, or otherwise modifying an immune response.

“Treatment” or “therapy” of a subject refers to any type of intervention or process performed on, or the administration of an active agent to, the subject with the objective of reversing, alleviating, ameliorating, inhibiting, slowing down, or preventing the onset, progression, development, severity, or recurrence of a symptom, complication, condition, or biochemical indicia associated with a disease.

A “subject” includes any human or non-human animal. The term “nonhuman animal” includes, but is not limited to, vertebrates such as nonhuman primates, sheep, dogs, and rodents such as mice, rats, and guinea pigs. In some embodiments, the subject is a human. The terms “subject” and “patient” and “individual” are used interchangeably herein.

A “therapeutically effective amount” or “therapeutically effective dosage” of a drug or therapeutic agent is any amount of the drug that, when used alone or in combination with another therapeutic agent, protects a subject against the onset of a disease or promotes disease regression evidenced by a decrease in severity of disease symptoms, an increase in frequency and duration of disease symptom-free periods, or a prevention of impairment or disability due to the disease affliction. The ability of a therapeutic agent to promote disease regression can be evaluated using a variety of methods known to the skilled practitioner, such as in human subjects during clinical trials, in animal model systems predictive of efficacy in humans, or by assaying the activity of the agent in in vitro assays.

As used herein, “subtherapeutic dose” means a dose of a therapeutic compound (e.g., an antibody) that is lower than the usual or typical dose of the therapeutic compound when administered alone for the treatment of a hyperproliferative disease (e.g., cancer).

By way of example, an “anti-cancer agent” promotes cancer regression in a subject. In some embodiments, a therapeutically effective amount of the drug promotes cancer regression to the point of eliminating the cancer. “Promoting cancer regression” means that administering an effective amount of the drug, alone or in combination with an anti-cancer agent, results in a reduction in tumor growth or size, necrosis of the tumor, a decrease in severity of at least one disease symptom, an increase in frequency and duration of disease symptom-free periods, or a prevention of impairment or disability due to the disease affliction. In addition, the terms “effective” and “effectiveness” with regard to a treatment includes both pharmacological effectiveness and physiological safety. Pharmacological effectiveness refers to the ability of the drug to promote cancer regression in the patient. Physiological safety refers to the level of toxicity or other adverse physiological effects at the cellular, organ and/or organism level (adverse effects) resulting from administration of the drug.

By way of example for the treatment of tumors, a therapeutically effective amount of an anti-cancer agent inhibits cell growth or tumor growth by at least about 10%, by at least about 20%, by at least about 30%, by at least about 40%, by at least about 50%, by at least about 60%, by at least about 70%, or by at least about 80%, by at least about 90%, at least about 95%, or at least about 100% relative to untreated subjects.

In other embodiments of the disclosure, tumor regression can be observed and continue for a period of at least about 20 days, at least about 30 days, at least about 40 days, at least about 50 days, or at least about 60 days. Notwithstanding these ultimate measurements of therapeutic effectiveness, evaluation of immunotherapeutic drugs must also make allowance for “immune-related response patterns”.

An “immune-related response pattern” refers to a clinical response pattern often observed in cancer patients treated with immunotherapeutic agents that produce antitumor effects by inducing cancer-specific immune responses or by modifying native immune processes. This response pattern is characterized by a beneficial therapeutic effect that follows an initial increase in tumor burden or the appearance of new lesions, which in the evaluation of traditional chemotherapeutic agents would be classified as disease progression and would be synonymous with drug failure. Accordingly, proper evaluation of immunotherapeutic agents can require long-term monitoring of the effects of these agents on the target disease.

“Sustained response” refers to the sustained effect on reducing tumor growth after cessation of a treatment. For example, the tumor size may remain to be the same or smaller as compared to the size at the beginning of the administration phase. In some embodiments, the sustained response has a duration at least the same as the treatment duration, at least 1.5×, 2. OX, 2.5×, or 3. OX length of the treatment duration.

As used herein, “complete response” or “CR” refers to disappearance of all target lesions; “partial response” or “PR” refers to at least a 30% decrease in the sum of the longest diameters (SLD) of target lesions, taking as reference the baseline SLD; and “stable disease” or “SD” refers to neither sufficient shrinkage of target lesions to qualify for PR, nor sufficient increase to qualify for PD, taking as reference the smallest SLD since the treatment started.

As used herein, “progression free survival” (PFS) refers to the length of time during and after treatment during which the disease being treated (e.g., cancer) does not get worse. Progression-free survival may include the amount of time patients have experienced a complete response or a partial response, as well as the amount of time patients have experienced stable disease.

As used herein, “overall response rate” (ORR) refers to the sum of complete response (CR) rate and partial response (PR) rate.

As used herein, “overall survival” refers to the percentage of individuals in a group who are likely to be alive after a particular duration of time.

A therapeutically effective amount of a drug includes a “prophylactically effective amount,” which is any amount of the drug that, when administered alone or in combination with an anti-cancer agent to a subject at risk of developing a cancer (e.g., a subject having a pre-malignant condition) or of suffering a recurrence of cancer, inhibits the development or recurrence of the cancer. In some embodiments, the prophylactically effective amount prevents the development or recurrence of the cancer entirely. “Inhibiting” the development or recurrence of a cancer means either lessening the likelihood of the cancer's development or recurrence, or preventing the development or recurrence of the cancer entirely.

The term “weight-based dose”, as referred to herein, means that a dose administered to a patient is calculated based on the weight of the patient. For example, when a patient with 60 kg body weight requires 3 mg/kg of an anti-CD30 antibody, one can calculate and use the appropriate amount of the anti-CD30 antibody (i.e., 180 mg) for administration.

The use of the term “flat dose” with regard to the methods and dosages of the disclosure means a dose that is administered to a patient without regard for the weight or body surface area (BSA) of the patient. The flat dose is therefore not provided as a mg/kg dose, but rather as an absolute amount of the agent (e.g., the anti-CD30 antibody). For example, a 60 kg person and a 100 kg person would receive the same dose of an antibody (e.g., 240 mg of an anti-CD30 antibody).

The phrase “pharmaceutically acceptable” indicates that the substance or composition must be compatible chemically and/or toxicologically, with the other ingredients comprising a formulation, and/or the mammal being treated therewith.

The phrase “pharmaceutically acceptable salt” as used herein, refers to pharmaceutically acceptable organic or inorganic salts of a compound of the invention. Exemplary salts include, but are not limited, to sulfate, citrate, acetate, oxalate, chloride, bromide, iodide, nitrate, bisulfate, phosphate, acid phosphate, isonicotinate, lactate, salicylate, acid citrate, tartrate, oleate, tannate, pantothenate, bitartrate, ascorbate, succinate, maleate, gentisinate, fumarate, gluconate, glucuronate, saccharate, formate, benzoate, glutamate, methanesulfonate “mesylate”, ethanesulfonate, benzenesulfonate, /?-toluenesulfonate, pamoate (i.e., I,Γ-methylene-bis-(2-hydroxy-3-naphthoate)) salts, alkali metal (e.g., sodiun and potassium) salts, alkaline earth metal (e.g., magnesium) salts, and ammonium salts. A pharmaceutically acceptable salt may involve the inclusion of another molecule such as an acetate ion, a succinate ion or other counter ion. The counter ion may be any organic or inorganic moiety that stabilizes the charge on the parent compound. Furthermore, a pharmaceutically acceptable salt may have more than one charged atom in its structure. Instances where multiple charged atoms are part of the pharmaceutically acceptable salt can have multiple counter ions. Hence, a pharmaceutically acceptable salt can have one or more charged atoms and/or one or more counter ion.

The use of the alternative (e.g., “or”) should be understood to mean either one, both, or any combination thereof of the alternatives. As used herein, the indefinite articles “a” or “an” should be understood to refer to “one or more” of any recited or enumerated component.

The terms “about” or “comprising essentially of” refer to a value or composition that is within an acceptable error range for the particular value or composition as determined by one of ordinary skill in the art, which will depend in part on how the value or composition is measured or determined, i.e., the limitations of the measurement system. For example, “about” or “comprising essentially of” can mean within 1 or more than 1 standard deviation per the practice in the art. Alternatively, “about” or “comprising essentially of” can mean a range of up to 20%. Furthermore, particularly with respect to biological systems or processes, the terms can mean up to an order of magnitude or up to 5-fold of a value. When particular values or compositions are provided in the application and claims, unless otherwise stated, the meaning of “about” or “comprising essentially of” should be assumed to be within an acceptable error range for that particular value or composition.

The terms “once about every week,” “once about every two weeks,” or any other similar dosing interval terms as used herein mean approximate numbers. “Once about every week” can include every seven days±one day, i.e., every six days to every eight days. “Once about every two weeks” can include every fourteen days±three days, i.e., every eleven days to every seventeen days. Similar approximations apply, for example, to once about every three weeks, once about every four weeks, once about every five weeks, once about every six weeks, and once about every twelve weeks. In some embodiments, a dosing interval of once about every six weeks or once about every twelve weeks means that the first dose can be administered any day in the first week, and then the next dose can be administered any day in the sixth or twelfth week, respectively. In other embodiments, a dosing interval of once about every six weeks or once about every twelve weeks means that the first dose is administered on a particular day of the first week (e.g., Monday) and then the next dose is administered on the same day of the sixth or twelfth weeks (i.e., Monday), respectively.

As described herein, any concentration range, percentage range, ratio range, or integer range is to be understood to include the value of any integer within the recited range and, when appropriate, fractions thereof (such as one tenth and one hundredth of an integer), unless otherwise indicated.

Various aspects of the disclosure are described in further detail in the following subsections.

### II. Methods of the Invention

In one aspect, the methods disclosed herein are used in place of standard of care therapies. The anti-CD30 antibody-drug conjugates described herein are used to decrease the activity of CD30⁺ T regulatory cells and/or increase the ratio of CD8⁺ T cells to CD30⁺ T regulatory cells in subjects having cancer, which can result in improved treatment compared to standard of care therapies. In certain embodiments, a standard of care therapy is used in combination with any method disclosed herein. Standard-of-care therapies for different types of cancer are well known by persons of skill in the art. For example, the National Comprehensive Cancer Network (NCCN), an alliance of 21 major cancer centers in the USA, publishes the NCCN Clinical Practice Guidelines in Oncology (NCCN GUIDELINES®) that provide detailed up-to-date information on the standard-of-care treatments for a wide variety of cancers (see NCCN GUIDELINES®, 2014, available at: www.nccn.org/professionals/physician_gls/f_guidelines.asp, last accessed May 14, 2014).

In some embodiments, the therapy of the present disclosure can be used to treat a lymphoma (e.g., a tumor derived from a lymphoma). Lymphoma is a form of cancer that affects the immune system. The majority of lymphomas fall within two categories: Hodgkin lymphoma (HL) and non-Hodgkin lymphoma (NHL). NHL is the most common form of lymphoma, accounting for about 90% of all cases of lymphoma, whereas HL accounts for only about 10% of all cases of lymphoma. Accordingly, in some embodiments of the methods provided herein, the lymphoma is an HL. In other embodiments of the methods provided herein, the lymphoma is an NHL.

NHL will account for an estimated 72,000 new cases (4.3% of all new cancer cases) and 20,000 deaths (3.4% of all cancer-related deaths) in the U.S. in 2017. See Howlader N et al., SEER Cancer Statistics Review, 1975-2014, based on November 2016 SEER data submission. Diffuse large B-cell lymphoma (DLBCL), the most common NHL subtype, has an incidence rate of 7.14 per 100,000 persons per year (P-Y), including up to 10% primary mediastinal B-cell lymphoma (PMBL). See Dunleavy K et al., Blood 2015; 125:33-39. Incidence rates of peripheral T-cell lymphoma (PTCL) and mycosis fungoides/Sézary syndrome (MF/SS) are 0.60 and 0.52 per 100,000 P-Y. See Morton L M et al., Blood 2006; 107:265-276. Within the two main categories of lymphoma, HL and NHL, there are several specific subgroups of lymphomas. Hodgkin lymphomas can include, but are not limited to, classical HL (cHL; e.g., nodular sclerosing HL, mixed cellularity HL, lymphocyte rich HL, and lymphocyte depleted HL) and nodular lymphocyte predominant type HL. Non-Hodgkin Lymphomas can include, but are not limited to, B-cell lymphomas (e.g., diffuse large B-cell lymphoma (DLBCL), follicular lymphoma (FL), Burkitt lymphoma, immunoblastic large cell lymphoma, precursor B-lymphoblastic lymphoma, and mantle cell lymphoma) and T cell lymphomas (e.g., a cutaneous T-cell lymphoma (CTCL), a peripheral T-cell lymphoma (PTCL), a mycosis fungoides, an anaplastic large cell lymphoma, and a precursor T-lymphoblastic lymphoma).

Treatment guidelines for relapsed/refractory (R/R) NHL recommend multi-agent chemotherapy (combined with targeted therapy for B-cell lymphomas), brentuximab vedotin (BV), autologous or allogeneic hematopoietic stem cell transplantation (HSCT), and/or radiotherapy, with addition of topical therapies for MF/SS. National Comprehensive Cancer Network, Non-Hodgkin Lymphoma (version 3.2016). 5-year relative survival rates are 48%, 44%, and 86% in DLBCL, PTCL, and MF/SS, respectively. SeeHan X et al., Cancer Causes Control 2008; 19:841-858.

A. Anti-CD30 Antibody-Drug Conjugates

In one aspect, the therapy of the present disclosure utilizes an anti-CD30 antibody or an antigen-binding fragment thereof. CD30 receptors are members of the tumor necrosis factor receptor superfamily involved in limiting the proliferative potential of autoreactive CD8 effector T cells. Antibodies targeting CD30 can potentially be either agonists or antagonists of these CD30 mediated activities.

Murine anti-CD30 mAbs known in the art have been generated by immunization of mice with Hodgkin's disease (HD) cell lines or purified CD30 antigen. AC10, originally termed C10 (Bowen et al., 1993, J. Immunol. 151:5896 5906), is distinct in that this anti-CD30 mAb that was prepared against a hum an NK-like cell line, YT (Bowen et al., 1993, J. Immunol. 151:5896 5906). Initially, the signaling activity of this mAb was evidenced by the down regulation of the cell surface expression of CD28 and CD45 molecules, the up regulation of cell surface CD25 expression and the induction of homotypic adhesion following binding of C10 to YT cells. Sequences of the AC10 antibody are set out in SEQ ID NO: 1-16 and Table A below. See also U.S. Pat. No. 7,090,843, incorporated herein by reference.

Generally, antibodies of the disclosure immunospecifically bind CD30 and exert cytostatic and cytotoxic effects on malignant cells in Hodgkin's disease. Antibodies of the disclosure are preferably monoclonal, and may be multispecific, human, humanized or chimeric antibodies, single chain antibodies, Fab fragments, F(ab′) fragments, fragments produced by a Fab expression library, and CD30 binding fragments of any of the above. The immunoglobulin molecules of the disclosure can be of any type (e.g., IgG, IgE, IgM, IgD, IgA and IgY), class (e.g., IgG1, IgG2, IgG3, IgG4, IgA1 and IgA2) or subclass of immunoglobulin molecule.

In certain embodiments of the disclosure, the antibodies are human antigen-binding antibody fragments of the present disclosure and include, but are not limited to, Fab, Fab′ and F(ab′)₂, Fd, single-chain Fvs (scFv), single-chain antibodies, disulfide-linked Fvs (sdFv) and fragments comprising either a V_{L} or V_{H} domain. Antigen-binding antibody fragments, including single-chain antibodies, may comprise the variable region(s) alone or in combination with the entirety or a portion of the following: hinge region, CH1, CH2, CH3 and CL domains. Also included in the disclosure are antigen-binding fragments also comprising any combination of variable region(s) with a hinge region, CH1, CH2, CH3 and CL domains. Preferably, the antibodies are human, murine (e.g., mouse and rat), donkey, sheep, rabbit, goat, guinea pig, camelid, horse, or chicken.

The antibodies of the present disclosure may be monospecific, bispecific, trispecific or of greater multi specificity. Multispecific antibodies may be specific for different epitopes of CD30 or may be specific for both CD30 as well as for a heterologous protein. See, e.g., PCT publications WO 93/17715; WO 92/08802; WO 91/00360; WO 92/05793; Tutt, et al., 1991, J. Immunol. 147:60 69; U.S. Pat. Nos. 4,474,893; 4,714,681; 4,925,648; 5,573,920; 5,601,819; Kostelny et al., 1992, J. Immunol. 148:1547 1553.

Antibodies of the present disclosure may be described or specified in terms of the particular CDRs they comprise. In certain embodiments antibodies of the disclosure comprise one or more CDRs of AC10. The disclosure encompasses an antibody or derivative thereof comprising a heavy or light chain variable domain, said variable domain comprising (a) a set of three CDRs, in which said set of CDRs are from monoclonal antibody AC10, and (b) a set of four framework regions, in which said set of framework regions differs from the set of framework regions in monoclonal antibody AC 10, and in which said antibody or derivative thereof immunospecifically binds CD30.

In one aspect, the anti-CD30 antibody is AC10. In some embodiments, the anti-CD30 antibody is cAC10. cAC10 is a chimeric IgG1 monoclonal antibody that specifically binds CD30. cAC10 induces growth arrest of CD30⁺ cell lines in vitro and has pronounced antitumor activity in severe combined immunodeficiency (SCID) mouse xenograft models of Hodgkin disease. See Francisco et al., Blood 102(4):1458-64 (2003). AC10 antibody and cAC10 antibody are described in U.S. Pat. Nos. 9,211,319, 7,090,843.

In one aspect, anti-CD30 antibodies that compete with AC10 antibody and/or cAC10 antibody binding to CD30 are provided. Anti-CD30 antibodies that bind to the same epitope as AC10 antibody and cAC10 antibody are also provided.

In one aspect, provided herein is an anti-CD30 antibody comprising 1, 2, 3, 4, 5, or 6 of the CDR sequences of the AC10 antibody. In one aspect, provided herein is an anti-CD30 antibody comprising 1, 2, 3, 4, 5, or 6 of the CDR sequences of the cAC10 antibody. In some embodiments, the CDR is a Kabat CDR or a Chothia CDR.

In one aspect, provided herein is an anti-CD30 antibody comprising a heavy chain variable region and a light chain variable region, wherein the heavy chain variable region comprises (i) CDR-H1 comprising the amino acid sequence of SEQ ID NO:1, (ii) CDR-H2 comprising the amino acid sequence of SEQ ID NO:2, and (iii) CDR-H3 comprising the amino acid sequence of SEQ ID NO:3; and/or wherein the light chain variable region comprises (i) CDR-L1 comprising the amino acid sequence of SEQ ID NO:4, (ii) CDR-L2 comprising the amino acid sequence of SEQ ID NO:5, and (iii) CDR-L3 comprising the amino acid sequence of SEQ ID NO:6.

An anti-CD30 antibody described herein may comprise any suitable framework variable domain sequence, provided that the antibody retains the ability to bind CD30 (e.g., human CD30). As used herein, heavy chain framework regions are designated “HC-FR1-FR4,” and light chain framework regions are designated “LC-F1FR4.” In some embodiments, the anti-CD30 antibody comprises a heavy chain variable domain framework sequence of SEQ ID NO:9, 10, 11, and 12 (HC-FR1, HC-FR2, HC-FR3, and HC-FR4, respectively). In some embodiments, the anti-CD30 antibody comprises a light chain variable domain framework sequence of SEQ ID NO:13, 14, 15, and 16 (LC-FR1, LC-FR2, LC-FR3, and LC-FR4, respectively).

In one embodiment, an anti-CD30 antibody comprises a heavy chain variable domain comprising a framework sequence and hypervariable regions, wherein the framework sequence comprises the HC-FR1-HC-FR4 amino acid sequences of SEQ ID NO:9 (HC-FR1), SEQ ID NO:10 (HC-FR2), SEQ ID NO:11 (HC-FR3), and SEQ ID NO:12 (HC-FR4), respectively; the CDR-H1 comprises the amino acid sequence of SEQ ID NO:1; the CDR-H2 comprises the amino acid sequence of SEQ ID NO:2; and the CDR-H3 comprises the amino acid sequence of SEQ ID NO:3.

In one embodiment, an anti-CD30 antibody comprises a light chain variable domain comprising a framework sequence and hypervariable regions, wherein the framework sequence comprises the LC-FR1-LC-FR4 amino acid sequences of SEQ ID NO:13 (LC-FR1), SEQ ID NO:14 (LC-FR2), SEQ ID NO:15 (LC-FR3), and SEQ ID NO:16 (LC-FR4), respectively; the CDR-L1 comprises the amino acid sequence of SEQ ID NO:4; the CDR-L2 comprises the amino acid sequence of SEQ ID NO:5; and the CDR-L3 comprises the amino acid sequence of SEQ ID NO:6.

In some embodiments of the anti-CD30 antibodies described herein, the heavy chain variable domain comprises the amino acid sequence of QIQLQQSGPEVVKPGASVKISCKASGYTFTDYYITWVKQKPGQGLEWIGWIYPGSGN TKYNEKFKGKATLTVDTSSSTAFMQLSSLTSEDTAVYFCANYGNYWFAYWGQGTQ VTVSA (SEQ ID NO:7) and the light chain variable domain comprises the amino acid sequence of DIVLTQSPASLAVSLGQRATISCKASQSVDFDGDSYMNWYQQKPGQPPKVLIYAASN LESGIPARFSGSGSGTDFTLNIHPVEEEDAATYYCQQSNEDPWTFGGGTKLEIK (SEQ ID NO:8).

In some embodiments of the anti-CD30 antibodies described herein, the heavy chain CDR sequences comprise the following:

a) CDR-H1
(DYYIT (SEQ ID NO: 1));
b) CDR-H2
(WIYPGSGNTKYNEKFKG (SEQ ID NO: 2));
and
c) CDR-H3
(YGNYWFAY (SEQ ID NO: 3)).

In some embodiments of the anti-CD30 antibodies described herein, the heavy chain FR sequences comprise the following:

a) HC-FR1
(QIQLQQSGPEVVKPGASVKISCKASGYTFT (SEQ ID NO: 9));
b) HC-FR2
(WVKQKPGQGLEWIG (SEQ ID NO: 10));
c) HC-FR3
(KATLTVDTSSSTAFMQLSSLTSEDTAVYFCAN (SEQ ID NO: 11));
and
d) HC-FR4
(WGQGTQVTVSA (SEQ ID NO: 12)).

In some embodiments of the anti-CD30 antibodies described herein, the light chain CDR sequences comprise the following:

a) CDR-L1
(KASQSVDFDGDSYMN (SEQ ID NO: 4));
b) CDR-L2
(AASNLES (SEQ ID NO: 5));
and
c) CDR-L3
(QQSNEDPWT (SEQ ID NO: 6)).

In some embodiments of the anti-CD30 antibodies described herein, the light chain FR sequences comprise the following:

a) LC-FR1
(DIVLTQSPASLAVSLGQRATISC (SEQ ID NO: 13));
b) LC-FR2
(WYQQKPGQPPKVLIY (SEQ ID NO: 14));
c) LC-FR3
(GIPARFSGSGSGTDFTLNIHPVEEEDAATYYC (SEQ ID NO: 15));
and
d) LC-FR4
(FGGGTKLEIK (SEQ ID NO: 16)).

In some embodiments, provided herein is an anti-CD30 antibody that binds to CD30 (e.g., human CD30), wherein the antibody comprises a heavy chain variable region and a light chain variable region, wherein the antibody comprises:
(a) heavy chain variable domain comprising:
(1) an HC-FR1 comprising the amino acid sequence of SEQ ID NO:9;
(2) an CDR-H1 comprising the amino acid sequence of SEQ ID NO:1;
(3) an HC-FR2 comprising the amino acid sequence of SEQ ID NO:10;
(4) an CDR-H2 comprising the amino acid sequence of SEQ ID NO:2;
(5) an HC-FR3 comprising the amino acid sequence of SEQ ID NO: 11;
(6) an CDR-H3 comprising the amino acid sequence of SEQ ID NO:3; and
(7) an HC-FR4 comprising the amino acid sequence of SEQ ID NO:12, and/or
(b) a light chain variable domain comprising:
(1) an LC-FR1 comprising the amino acid sequence of SEQ ID NO:13;
(2) an CDR-L1 comprising the amino acid sequence of SEQ ID NO:4;
(3) an LC-FR2 comprising the amino acid sequence of SEQ ID NO:14;
(4) an CDR-L2 comprising the amino acid sequence of SEQ ID NO:5;
(5) an LC-FR3 comprising the amino acid sequence of SEQ ID NO:15;
(6) an CDR-L3 comprising the arnino acid sequence of SEQ ID NO:6; and
(7) an LC-FR4 comprising the amino acid sequence of SEQ ID NO:16.

In one aspect, provided herein is an anti-CD30 antibody comprising a heavy chain variable domain comprising the amino acid sequence of SEQ ID NO:7 and/or comprising a light chain variable domain comprising the amino acid sequence of SEQ ID NO:8.

In some embodiments, provided herein is an anti-CD30 antibody comprising a heavy chain variable domain comprising an amino acid sequence having at least 85%, 86%, 87%, 88%, 89%, 90%, 91%, 92%, 93%, 94%, 95%, 96%, 97%, 98%, or 99% sequence identity to the amino acid sequence of SEQ ID NO:7. In certain embodiments, a heavy chain variable domain comprising an amino acid sequence having at least 85%, 86%, 87%, 88%, 89%, 90%, 91%, 92%, 93%, 94%, 95%, 96%, 97%, 98%, or 99% sequence identity to the amino acid sequence of SEQ ID NO:7 contains substitutions (e.g., conservative substitutions), insertions, or deletions relative to the reference sequence and retains the ability to bind to a CD30 (e.g., human CD30). In certain embodiments, a total of 1 to 10 amino acids have been substituted, inserted and/or deleted in SEQ ID NO:7. In certain embodiments, substitutions, insertions, or deletions (e.g., 1, 2, 3, 4, or 5 amino acids) occur in regions outside the CDR s (i.e., in the FRs). In some embodiments, the anti-CD30 antibody comprises a heavy chain variable domain sequence of SEQ ID NO:7 including post-translational modifications of that sequence. In a particular embodiment, the heavy chain variable domain comprises one, two or three CDRs selected from: (a) CDR-H1 comprising the amino acid sequence of SEQ ID NO:1, (b) CDR-H2 comprising the amino acid sequence of SEQ ID NO:2, and (c) CDR-H3 comprising the amino acid sequence of SEQ ID NO:3.

In some embodiments, provided herein is an anti-CD30 antibody comprising a light chain variable domain comprising an amino acid sequence having at least 85%, 86%, 87%, 88%, 89%, 90%, 91%, 92%, 93%, 94%, 95%, 96%, 97%, 98%, or 99% sequence identity to the amino acid sequence of SEQ ID NO:8. In certain embodiments, a light chain variable domain comprising an amino acid sequence having at least 85%, 86%, 87%, 88%, 89%, 90%, 91%, 92%, 93%, 94%, 95%, 96%, 97%, 98%, or 99% sequence identity to the amino acid sequence of SEQ ID NO:8 contains substitutions (e.g., conservative substitutions), insertions, or deletions relative to the reference sequence and retains the ability to bind to a CD30 (e.g., human CD30). In certain embodiments, a total of 1 to 10 amino acids have been substituted, inserted and/or deleted in SEQ ID NO:8. In certain embodiments, substitutions, insertions, or deletions (e.g., 1, 2, 3, 4, or 5 amino acids) occur in regions outside the CDR s (i.e., in the FRs). In some embodiments, the anti-CD30 antibody comprises a light chain variable domain sequence of SEQ ID NO:8 including post-translational modifications of that sequence. In a particular embodiment, the light chain variable domain comprises one, two or three CDRs selected from: (a) CDR-H1 comprising the amino acid sequence of SEQ ID NO:4, (b) CDR-H2 comprising the amino acid sequence of SEQ ID NO:5, and (c) CDR-H3 comprising the amino acid sequence of SEQ ID NO:6.

In some embodiments, the anti-CD30 antibody comprises a heavy chain variable domain as in any of the embodiments provided above, and a light chain variable domain as in any of the embodiments provided above. In one embodiment, the antibody comprises the heavy chain variable domain sequence of SEQ ID NO:7 and the light chain variable domain sequence of SEQ ID NO:8, including post-translational modifications of those sequences.

In some embodiments, the anti-CD30 antibody of the anti-CD30 antibody-drug conjugate comprises: i) a heavy chain CDR1 set out in SEQ ID NO: 1, a heavy chain CDR2 set out in SEQ ID NO: 2, a heavy chain CDR3 set out in SEQ ID NO: 3; and ii) a light chain CDR1 set out in SEQ ID NO: 4, a light chain CDR2 set out in SEQ ID NO: 5, and a light chain CDR3 set out in SEQ ID NO: 6.

In some embodiments, the anti-CD30 antibody of the anti-CD30 antibody-drug conjugate comprises: i) an amino acid sequence at least 85% identical to a heavy chain variable region set out in SEQ ID NO: 7, and ii) an amino acid sequence at least 85% identical to a light chain variable region set out in SEQ ID NO: 8.

In some embodiments, the anti-CD30 antibody of the anti-CD30 antibody-drug conjugate is a monoclonal antibody.

In some embodiments, the anti-CD30 antibody of the anti-CD30 antibody-drug conjugate is a chimeric AC10 antibody.

Antibodies of the present invention may also be described or specified in terms of their binding affinity to CD30. Preferred binding affinities include those with a dissociation constant or Kd less than 5×10² M, 10⁻² M, 5×10⁻³ M, 10⁻³ M, 5×10⁻⁴ M, 10⁻⁴ M, 5×10⁻⁵ M, 10⁻⁵ M, 5×10⁻⁶ M, 10⁻⁶ M, 5×10⁻⁷ M, 10⁻⁷ M, 5×10⁻⁸ M, 10⁻⁸M, 5×10⁻⁹ M, 10⁻⁹ M, 5×10⁻¹⁰ M, 10⁻¹⁰ M, 5×10⁻¹¹ M, 10⁻¹¹ M, 5×10⁻¹² M, 10⁻¹² M, 5×10⁻¹³ M, 10⁻¹³ M, 5×10⁻¹⁴ M, 10⁻¹⁴ M, 5×10⁻¹⁵ M, or 10⁻¹⁵ M.

There are five classes of immunoglobulins: IgA, IgD, IgE, IgG and IgM, having heavy chains designated α, δ, ε, γ and μ, respectively. The γ and α classes are further divided into subclasses e.g., humans express the following subclasses: IgG1, IgG2, IgG3, IgG4, IgA1 and IgA2. IgG1 antibodies can exist in multiple polymorphic variants termed allotypes (reviewed in Jefferis and Lefranc 2009. mAbs Vol 1 Issue 4 1-7) any of which are suitable for use in some of the embodiments herein. Common allotypic variants in human populations are those designated by the letters a, f, n, z or combinations thereof. In any of the embodiments herein, the antibody may comprise a heavy chain Fc region comprising a human IgG Fc region. In further embodiments, the human IgG Fc region comprises a human IgG1.

In one aspect of the invention, polynucleotides encoding anti-CD30 antibodies, such as those anti-CD30 antibodies described herein, are provided. In certain embodiments, vectors comprising polynucleotides encoding anti-CD30 antibodies as described herein are provided. In certain embodiments, host cells comprising such vectors are provided. In another aspect of the invention, compositions comprising anti-CD30 antibodies described herein or polynucleotides encoding anti-CD30 antibodies described herein are provided.

The antibodies also include derivatives that are modified, i.e., by the covalent attachment of any type of molecule to the antibody such that covalent attachment does not prevent the antibody from binding to CD30 or from exerting a cytostatic or cytotoxic effect on HD cells. For example, but not by way of limitation, the antibody derivatives include antibodies that have been modified, e.g., by glycosylation, acetylation, PEGylation, phosphylation, amidation, derivatization by known protecting/blocking groups, proteolytic cleavage, linkage to a cellular ligand or other protein, etc. Any of numerous chemical modifications may be carried out by known techniques, including, but not limited to specific chemical cleavage, acetylation, formylation, metabolic synthesis of tunicamycin, etc. Additionally, the derivative may contain one or more non-classical amino acids.

In some embodiments, the anti-CD30 antibody is conjugated to a therapeutic agent (e.g., an anti-CD30 antibody-drug conjugate). In some embodiments, the therapeutic agent comprises an anti-neoplastic agent (e.g., an anti-mitotic agent). In certain embodiments, the therapeutic agent is selected from the group consisting of monomethyl auristatin E (MMAE), monomethyl auristatin F (MMAF), auristatin drug analogues, cantansinoids, maytansinoids (e.g., maytansine; DMs), dolastatins, cryptophycin, duocarmycin, duocarmycin derivatives, esperamicin, calicheamicin, pyrolobenodiazepine (PBD), and any combination thereof. In one particular embodiment, the anti-CD30 antibody is conjugated to MMAE. The antibody can be conjugated to at least one, at least two, at least three, at least four, at least five, at least six, at least seven, at least eight, at least nine, or at least ten molecules of the therapeutic agent (e.g., MMAE). In one embodiment, the anti-CD30 antibody is conjugated to four molecules of the therapeutic agent, e.g., four molecules of MMAE. In one particular embodiment, the anti-CD30 antibody is conjugated to MMAF. The antibody can be conjugated to at least one, at least two, at least three, at least four, at least five, at least six, at least seven, at least eight, at least nine, or at least ten molecules of the therapeutic agent (e.g., MMAF). In one embodiment, the anti-CD30 antibody is conjugated to four molecules of the therapeutic agent, e.g., four molecules of MMAF.

In some embodiments, the anti-CD30 antibody-drug conjugate further comprises a linker between the therapeutic agent and the antibody. In some embodiments, the linker comprises one or more naturally occurring amino acids, one or more non-naturally occurring (e.g., synthetic) amino acids, a chemical linker, or any combination thereof. In certain embodiments, the linker is a cleavable linker, e.g., a protease cleavable linker. In certain embodiments, the linker is specifically cleaved upon uptake by a target cell, e.g., upon uptake by a cell expressing CD30. In certain embodiments, the linker is a cleavable peptide linker having the formula: “-MC-vc-PAB-” or “-MC-val-cit-PAB-”, wherein “MC” refers to the stretcher maleimidocaproyl having the following structure:

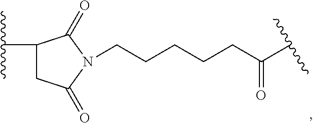 [SMILES: *C(=O)CCCCCN1C(=O)CC(*)C1=O]

“vc” and “val-cit” refer to the dipeptide valine-citrulline, and PAB refers to a self-immolative spacer having the following structure:

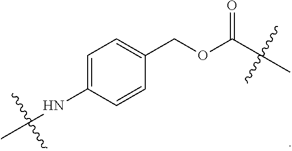 [SMILES: *Nc1ccc(COC(*)=O)cc1]

In some embodiments, cleavage of the linker activates a cytotoxic activity of the therapeutic agent. In certain embodiments, the linker is a non-cleavable linker. In certain embodiments, the non-cleavable linker has the formula: “-MC-”, wherein “MC” refers to the stretcher maleimidocaproyl having the following structure:

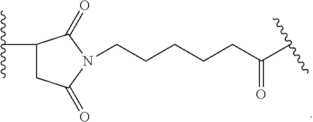 [SMILES: *C(=O)CCCCCN1C(=O)CC(*)C1=O]

In some embodiments, the antibody-drug conjugates comprises an anti-CD30 antibody, covalently linked to MMAE through a vc-PAB linker. In some embodiments, the antibody-drug conjugate is delivered to the subject as a pharmaceutical composition. In some embodiments, the CD30 antibody drug conjugates contemplated herein are as described in U.S. Pat. No. 9,211,319, herein incorporated by reference.

In one embodiment, the anti-CD30 antibody drug-conjugate comprises brentuximab vedotin. In one particular embodiment, the anti-CD30 antibody drug-conjugate is brentuximab vedotin. Brentuximab vedotin (BV; also known as “ADCETRIS®”) is a CD30-directed antibody-drug conjugate (ADC) comprising a chimeric anti-CD30 antibody (cAC10), a therapeutic agent (MMAE), and a protease-cleavable linker between the cAC10 and the MMAE, as shown in the following structure:

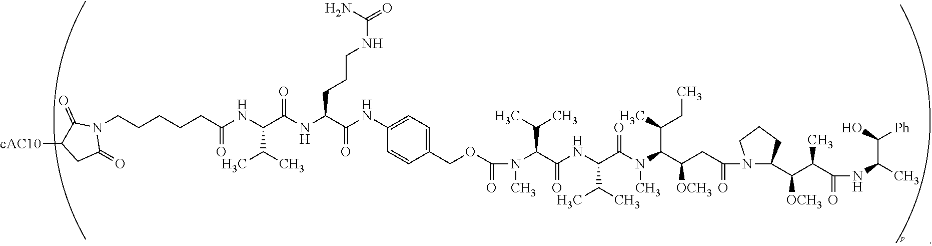

The drug to antibody ratio or drug loading is represented by “p” in the structure of brentuximab vedotin and ranges in integer values from 1 to 8. The average drug loading of brentuximab vedotin in a pharmaceutical composition is about 4. ADCETRIS® is approved by the FDA for treatment of patients with Hodgkin lymphoma after failure of autologous stem cell transplant (ASCT) or after failure of at least two prior multi-agent chemotherapy regimens in patients who are not ASCT candidates and for the treatment of patients with systemic anaplastic large cell lymphoma after failure of at least one prior multi-agent chemotherapy regimen.

In one embodiment, the anti-CD30 antibody is an anti-CD30 antibody or antigen-binding fragment thereof that binds to the same epitope as cAC10, e.g., the same epitope as brentuximab vedotin. In certain embodiments, the anti-CD30 antibody is an antibody that has the same CDRs as cAC10, e.g., the same CDRs as brentuximab vedotin. Antibodies that bind to the same epitope are expected to have functional properties very similar to those of cAC10 by virtue of their binding to the same epitope region of CD30. These antibodies can be readily identified based on their ability to, for example, cross-compete with cAC10 in standard CD30 binding assays such as Biacore analysis, ELISA assays, or flow cytometry.

In certain embodiments, the antibodies that cross-compete for binding to human CD30 with, or bind to the same epitope region of human CD30 as cAC10 are monoclonal antibodies. For administration to human subjects, these cross-competing antibodies can be chimeric antibodies, or can be humanized or human antibodies. Such chimeric, humanized, or human monoclonal antibodies can be prepared and isolated by methods well known in the art. Anti-CD30 antibodies usable in the methods of the disclosed disclosure also include antigen-binding portions of the above antibodies.

In other embodiments, the anti-CD30 antibody or antigen-binding portion thereof is a chimeric, humanized, or human monoclonal antibody or a portion thereof. In certain embodiments for treating a human subject, the antibody is a humanized antibody. In other embodiments for treating a human subject, the antibody is a human antibody. Antibodies of an IgG1, IgG2, IgG3, or IgG4 isotype can be used.

B. Methods of Modulating the Immune Response

In one aspect, the present invention provides for a method of decreasing the activity of CD30⁺ T regulatory (Treg) cells in a subject having cancer comprising administering to the subject an antibody-drug conjugate, wherein the antibody-drug conjugate comprises an anti-CD30 antibody or an antigen-binding portion thereof conjugated to a monomethyl auristatin.

In some embodiments, decreasing the activity of CD30⁺ Treg cells comprises a decrease in the number of CD30⁺ Treg cells. In some embodiments, the number of CD30⁺ Treg cells is decreased relative to the number of one or more other types of CD4⁺ T cells. In some embodiments, the one or more other types of CD4⁺ T cells comprise Th1 cells, Th2 cells or Th17 cells. In some embodiments, the one or more other types of CD4⁺ T cells comprise Th1 CD30⁺ cells, Th2 CD30⁺ cells or Th17 CD30⁺ cells. In some embodiments, the number of CD30⁺ Treg cells is decreased relative to the number of CD30⁺ Treg cells in the subject prior to administration of the antibody-drug conjugate. In some embodiments, number of CD30⁺ Treg cells is decreased relative to the number of CD30⁺ Treg cells in a subject who has not been treated with the antibody-drug conjugate.

In some embodiments, decreasing the activity of CD30⁺ Treg cells comprises a decrease in the function of CD30⁺ Treg cells. In some embodiments, the decrease in the function of CD30⁺ Treg cells is relative to the function of CD30⁺ Treg cells in a subject prior to administration of the antibody-drug conjugate. In some embodiments, the decrease in the function of CD30⁺ Treg cells is relative to the function of CD30⁺ Treg cells in a subject who has not been treated with the antibody-drug conjugate.

In some embodiments, the CD30⁺ Treg cells are CD30⁺ inducible T regulatory (iTreg) cells or CD30⁺ peripheral T regulatory (pTreg) cells.

In some embodiments, the monomethyl auristatin is monomethyl auristatin E (MMAE). In some embodiments, the monomethyl auristatin is monomethyl auristatin F (MMAF).

In some embodiments, the anti-CD30 antibody is monoclonal anti-CD30 antibody AC10. In some embodiments, the anti-CD30 antibody is cAC10. In some embodiments, the antibody-drug conjugate is brentuximab vedotin.

In some embodiments, the anti-CD30 antibody comprises a heavy chain variable region and a light chain variable region, wherein the heavy chain variable region comprises:
(i) a CDR-H1 comprising the amino acid sequence of SEQ ID NO: 1;
(ii) a CDR-H2 comprising the amino acid sequence of SEQ ID NO: 2; and
(iii) a CDR-H3 comprising the amino acid sequence of SEQ ID NO: 3; and
   wherein the light chain variable region comprises:
(i) a CDR-L1 comprising the amino acid sequence of SEQ ID NO: 4;
(ii) a CDR-L2 comprising the amino acid sequence of SEQ ID NO: 5; and
(iii) a CDR-L3 comprising the amino acid sequence of SEQ ID NO: 6.

In some embodiments, the anti-CD30 antibody comprises a heavy chain variable region comprising the amino acid sequence of SEQ ID NO: 7 and a light chain variable region comprising the amino acid sequence of SEQ ID NO: 8.

In some embodiments, the antibody-drug conjugate further comprises a linker between the anti-CD30 antibody or antigen-binding portion thereof and the monomethyl auristatin. In some embodiments, the linker is selected from the group consisting of a cleavable linker and a non-cleavable linker. In some embodiments, the linker is a cleavable peptide linker. In some embodiments, the cleavable peptide linker has a formula: -MC-vc-PAB-. In some embodiments, the linker is a non-cleavable linker having a formula: -MC-.

In some embodiments, the subject has been previously treated for the cancer. In some embodiments, the subject did not respond to treatment or relapsed after first-line treatment. In some embodiments, the subject has not previously been treated for the cancer.

In some embodiments, the cancer is a lymphoma. In some embodiments, the lymphoma is a T-cell lymphoma. In some embodiments, the lymphoma is a B-cell lymphoma.

In some embodiments, the lymphoma is a non-Hodgkin lymphoma. In some embodiments, the subject has been previously treated for the non-Hodgkin lymphoma and the subject did not respond to treatment or relapsed after first-line treatment. In some embodiments, the subject has not been previously treated for the non-Hodgkin lymphoma. In some embodiments, the non-Hodgkin lymphoma is a mature T-cell lymphoma. In some embodiments, the non-Hodgkin lymphoma is diffuse large B-cell lymphoma (DLBCL), peripheral T-cell lymphoma (PTCL), anaplastic large cell lymphoma (ALCL) or cutaneous T-cell lymphoma (CTCL). In some embodiments, the non-Hodgkin lymphoma is cutaneous T-cell lymphoma (CTCL). In some embodiments, the cutaneous T-cell lymphoma (CTCL) is a mycosis fungoides (MF). In some embodiments, the mycosis fungoides is a CD30-positive mycosis fungoides (MF). In some embodiments, the cutaneous T-cell lymphoma (CTCL) is a primary cutaneous anaplastic large cell lymphoma (pcALCL). In some embodiments, the subject has received prior systemic treatment. In some embodiments, the non-Hodgkin lymphoma is anaplastic large cell lymphoma (ALCL). In some embodiments, the anaplastic large cell lymphoma (ALCL) is a systemic anaplastic large cell lymphoma (sALCL).

In some embodiments, the lymphoma is a Hodgkin lymphoma. In some embodiments, the subject has been previously treated for the Hodgkin lymphoma and the subject did not respond to treatment or relapsed after first-line treatment. In some embodiments, the subject relapsed after autologous stem cell transplant. In some embodiments, the subject relapsed after first-line treatment and the subject is ineligible for autologous stem cell transplant. In some embodiments, the subject has not been previously treated for the Hodgkin lymphoma. In some embodiments, the Hodgkin lymphoma is classical Hodgkin lymphoma (cHL). In some embodiments, the classical Hodgkin lymphoma (cHL) is advanced cHL. In some embodiments, the subject has been previously treated for cHL. In some embodiments, the subject has not been previously treated for cHL.

In some embodiments, the method further comprises administering one or more additional therapeutic agents capable of modulating the immune response. In some embodiments, the one or more additional therapeutic agents is not an antibody or antigen-binding fragment thereof. In some embodiments, the one or more additional therapeutic agents is an antibody or antigen-binding fragment thereof.

In some embodiments, the method further comprises administering one or more additional therapeutic agents. In some embodiments, the one or more additional therapeutic agents is a chemotherapy regimen consisting essentially of doxorubicin, vinblastine, and dacarbazine (AVD). In some embodiments, the one or more additional therapeutic agents is a chemotherapy regimen consisting essentially of Cyclophosphamide, Doxorubicin, and Prednisone (CHP). In some embodiments, the one or more additional therapeutic agents is an alkylating agent, an anthracycline, an antibiotic, an antifolate, an antimetabolite, an antitubulin agent, an auristatin, a chemotherapy sensitizer, a DNA minor groove binder, a DNA replication inhibitor, a duocarmycin, an etoposide, a fluorinated pyrimidine, a lexitropsin, a nitrosourea, a platinol, a purine antimetabolite, a puromycin, a radiation sensitizer, a steroid, a taxane, a topoisomerase inhibitor, and/or a vinca alkaloid. In some embodiments, the one or more additional therapeutic agents is selected from the group consisting of adriamycin, an androgen, anthramycin (AMC), asparaginase, 5-azacytidine, azathioprine, bleomycin, busulfan, buthionine sulfoximine, camptothecin, carboplatin, carmustine (BSNU), CC-1065, chlorambucil, cisplatin, colchicine, cyclophosphamide, cytarabine, cytidine arabinoside, cytochalasin B, dacarbazine, dactinomycin (formerly actinomycin), daunorubicin, decarbazine, docetaxel, doxorubicin, an estrogen, 5-fluordeoxyuridine, 5-fluorouracil, gramicidin D, hydroxydaunorubicin, hydroxyurea, idarubicin, ifosfamide, irinotecan, lomustine (CCNU), mechlorethamine, melphalan, 6-mercaptopurine, methotrexate, mithramycin, mitomycin C, mitoxantrone, nitroimidazole, paclitaxel, plicamycin, prednisone, prednisolone, procarbizine, streptozotocin, tenoposide, 6-thioguanine, thioTEPA, topotecan, vinblastine, vincristine, vinorelbine, VP-16 and VM-26. In some embodiments, the one or more additional therapeutic agents is an antibody or antigen-binding fragment thereof.

In some embodiments, the subject has cHL that has not been previously treated and the one or more additional therapeutic agents are adriamycin, dacarabazine and vinblastine. In some embodiments, the cHL is advanced cHL.

In some embodiments, the subject has a mature T-cell lymphoma that has not been previously treated and the one or more additional therapeutic agents are cyclophosphamide, hydroxydaunorubicin and prednisone.

In some embodiments, the subject has a mature T-cell lymphoma that has not been previously treated and the one or more additional therapeutic agents are cyclophosphamide, hydroxydaunorubicin and prednisolone.

In some embodiments, the method further comprises treating the subject with irradiation.

In another aspect, the present invention provides for a method of increasing the ratio of CD8⁺ T cells to CD30⁺ T regulatory (Treg) cells in a subject having cancer comprising administering to the subject an antibody-drug conjugate, wherein the antibody-drug conjugate comprises an anti-CD30 antibody or an antigen-binding portion thereof conjugated to a monomethyl auristatin. In some embodiments, the ratio of CD8⁺ T cells to CD30⁺ Treg cells is increased relative to the ratio of CD8⁺ T cells to CD30⁺ Treg cells in the subject prior to the administration of the antibody-drug conjugate.

In some embodiments, the CD30⁺ Treg cells are CD30⁺ inducible T regulatory (iTreg) cells or CD30⁺ peripheral T regulatory (pTreg) cells.

In some embodiments, the monomethyl auristatin is monomethyl auristatin E (MMAE). In some embodiments, the monomethyl auristatin is monomethyl auristatin F (MMAF).

In some embodiments, the anti-CD30 antibody is monoclonal anti-CD30 antibody AC10. In some embodiments, the anti-CD30 antibody is cAC10. In some embodiments, the antibody-drug conjugate is brentuximab vedotin.

In some embodiments, the anti-CD30 antibody comprises a heavy chain variable region and a light chain variable region, wherein the heavy chain variable region comprises:
(i) a CDR-H1 comprising the amino acid sequence of SEQ ID NO: 1;
(ii) a CDR-H2 comprising the amino acid sequence of SEQ ID NO: 2; and
(iii) a CDR-H3 comprising the amino acid sequence of SEQ ID NO: 3; and
   wherein the light chain variable region comprises:
(i) a CDR-L1 comprising the amino acid sequence of SEQ ID NO: 4;
(ii) a CDR-L2 comprising the amino acid sequence of SEQ ID NO: 5; and
(iii) a CDR-L3 comprising the amino acid sequence of SEQ ID NO: 6.

In some embodiments, the anti-CD30 antibody comprises a heavy chain variable region comprising the amino acid sequence of SEQ ID NO: 7 and a light chain variable region comprising the amino acid sequence of SEQ ID NO: 8.

In some embodiments, the antibody-drug conjugate further comprises a linker between the anti-CD30 antibody or antigen-binding portion thereof and the monomethyl auristatin. In some embodiments, the linker is selected from the group consisting of a cleavable linker and a non-cleavable linker. In some embodiments, the linker is a cleavable peptide linker. In some embodiments, the cleavable peptide linker has a formula: -MC-vc-PAB-. In some embodiments, the linker is a non-cleavable linker having a formula: -MC-.

In some embodiments, the subject has been previously treated for the cancer. In some embodiments, the subject did not respond to treatment or relapsed after first-line treatment. In some embodiments, the subject has not previously been treated for the cancer.

In some embodiments, the cancer is a lymphoma. In some embodiments, the lymphoma is a T-cell lymphoma. In some embodiments, the lymphoma is a B-cell lymphoma.

In some embodiments, the lymphoma is a non-Hodgkin lymphoma. In some embodiments, the subject has been previously treated for the non-Hodgkin lymphoma and the subject did not respond to treatment or relapsed after first-line treatment. In some embodiments, the subject has not been previously treated for the non-Hodgkin lymphoma. In some embodiments, the non-Hodgkin lymphoma is a mature T-cell lymphoma. In some embodiments, the non-Hodgkin lymphoma is diffuse large B-cell lymphoma (DLBCL), peripheral T-cell lymphoma (PTCL), anaplastic large cell lymphoma (ALCL) or cutaneous T-cell lymphoma (CTCL). In some embodiments, the non-Hodgkin lymphoma is cutaneous T-cell lymphoma (CTCL). In some embodiments, the cutaneous T-cell lymphoma (CTCL) is a mycosis fungoides (MF). In some embodiments, the mycosis fungoides is a CD30-positive mycosis fungoides (MF). In some embodiments, the cutaneous T-cell lymphoma (CTCL) is a primary cutaneous anaplastic large cell lymphoma (pcALCL). In some embodiments, the subject has received prior systemic treatment. In some embodiments, the non-Hodgkin lymphoma is anaplastic large cell lymphoma (ALCL). In some embodiments, the anaplastic large cell lymphoma (ALCL) is a systemic anaplastic large cell lymphoma (sALCL).

In some embodiments, the lymphoma is a Hodgkin lymphoma. In some embodiments, the subject has been previously treated for the Hodgkin lymphoma and the subject did not respond to treatment or relapsed after first-line treatment. In some embodiments, the subject relapsed after autologous stem cell transplant. In some embodiments, the subject relapsed after first-line treatment and the subject is ineligible for autologous stem cell transplant. In some embodiments, the subject has not been previously treated for the Hodgkin lymphoma. In some embodiments, the Hodgkin lymphoma is classical Hodgkin lymphoma (cHL). In some embodiments, the classical Hodgkin lymphoma (cHL) is advanced cHL. In some embodiments, the subject has been previously treated for cHL. In some embodiments, the subject has not been previously treated for cHL.

In some embodiments, the method further comprises administering one or more additional therapeutic agents capable of modulating the immune response. In some embodiments, the one or more additional therapeutic agents is not an antibody or antigen-binding fragment thereof. In some embodiments, the one or more additional therapeutic agents is an antibody or antigen-binding fragment thereof.

In some embodiments, the method further comprises administering one or more additional therapeutic agents. In some embodiments, the one or more additional therapeutic agents is a chemotherapy regimen consisting essentially of doxorubicin, vinblastine, and dacarbazine (AVD). In some embodiments, the one or more additional therapeutic agents is a chemotherapy regimen consisting essentially of Cyclophosphamide, Doxorubicin, and Prednisone (CHP). In some embodiments, the one or more additional therapeutic agents is an alkylating agent, an anthracycline, an antibiotic, an antifolate, an antimetabolite, an antitubulin agent, an auristatin, a chemotherapy sensitizer, a DNA minor groove binder, a DNA replication inhibitor, a duocarmycin, an etoposide, a fluorinated pyrimidine, a lexitropsin, a nitrosourea, a platinol, a purine antimetabolite, a puromycin, a radiation sensitizer, a steroid, a taxane, a topoisomerase inhibitor, and/or a vinca alkaloid. In some embodiments, the one or more additional therapeutic agents is selected from the group consisting of adriamycin, an androgen, anthramycin (AMC), asparaginase, 5-azacytidine, azathioprine, bleomycin, busulfan, buthionine sulfoximine, camptothecin, carboplatin, carmustine (BSNU), CC-1065, chlorambucil, cisplatin, colchicine, cyclophosphamide, cytarabine, cytidine arabinoside, cytochalasin B, dacarbazine, dactinomycin (formerly actinomycin), daunorubicin, decarbazine, docetaxel, doxorubicin, an estrogen, 5-fluordeoxyuridine, 5-fluorouracil, gramicidin D, hydroxydaunorubicin, hydroxyurea, idarubicin, ifosfamide, irinotecan, lomustine (CCNU), mechlorethamine, melphalan, 6-mercaptopurine, methotrexate, mithramycin, mitomycin C, mitoxantrone, nitroimidazole, paclitaxel, plicamycin, prednisone, prednisolone, procarbizine, streptozotocin, tenoposide, 6-thioguanine, thioTEPA, topotecan, vinblastine, vincristine, vinorelbine, VP-16 and VM-26. In some embodiments, the one or more additional therapeutic agents is an antibody or antigen-binding fragment thereof.

In some embodiments, the subject has cHL that has not been previously treated and wherein the one or more additional therapeutic agents are adriamycin, dacarabazine and vinblastine. In some embodiments, the cHL is advanced cHL.

In some embodiments, the subject has a mature T-cell lymphoma that has not been previously treated and wherein the one or more additional therapeutic agents are cyclophosphamide, hydroxydaunorubicin and prednisone.

In some embodiments, the subject has a mature T-cell lymphoma that has not been previously treated and wherein the one or more additional therapeutic agents are cyclophosphamide, hydroxydaunorubicin and prednisolone.

In some embodiments, the method further comprises treating the subject with irradiation.

In another aspect, the present invention provides for a method of modulating the immune response in a subject having cancer comprising administering to the subject an antibody-drug conjugate, wherein the antibody-drug conjugate comprises an anti-CD30 antibody or an antigen-binding portion thereof conjugated to a monomethyl auristatin, wherein the modulation comprises increasing the ratio of CD8⁺ T cells to CD30⁺ T regulatory (Treg) cells in the subject. In some embodiments, the ratio of CD8⁺ T cells to CD30⁺ Treg cells is increased relative to the ratio of CD8⁺ T cells to CD30⁺ Treg cells in the subject prior to the administration of the antibody-drug conjugate. In some embodiments, the ratio of CD8⁺ T cells to CD30⁺ Treg cells is increased relative to the ratio of CD8⁺ T cells to CD30⁺ Treg cells in a subject who has not been treated with the antibody-drug conjugate.

In some embodiments, the CD30⁺ Treg cells are CD30⁺ inducible T regulatory (iTreg) cells or CD30⁺ peripheral T regulatory (pTreg) cells.

In some embodiments, the monomethyl auristatin is monomethyl auristatin E (MMAE). In some embodiments, the monomethyl auristatin is monomethyl auristatin F (MMAF).

In some embodiments, the anti-CD30 antibody is monoclonal anti-CD30 antibody AC10. In some embodiments, the anti-CD30 antibody is cAC10. In some embodiments, the antibody-drug conjugate is brentuximab vedotin.

In some embodiments, the anti-CD30 antibody comprises a heavy chain variable region and a light chain variable region, wherein the heavy chain variable region comprises:
(i) a CDR-H1 comprising the amino acid sequence of SEQ ID NO: 1;
(ii) a CDR-H2 comprising the amino acid sequence of SEQ ID NO: 2; and
(iii) a CDR-H3 comprising the amino acid sequence of SEQ ID NO: 3; and
   wherein the light chain variable region comprises:
(i) a CDR-L1 comprising the amino acid sequence of SEQ ID NO: 4;
(ii) a CDR-L2 comprising the amino acid sequence of SEQ ID NO: 5; and
(iii) a CDR-L3 comprising the amino acid sequence of SEQ ID NO: 6.

In some embodiments, the anti-CD30 antibody comprises a heavy chain variable region comprising the amino acid sequence of SEQ ID NO: 7 and a light chain variable region comprising the amino acid sequence of SEQ ID NO: 8.

In some embodiments, the antibody-drug conjugate further comprises a linker between the anti-CD30 antibody or antigen-binding portion thereof and the monomethyl auristatin. In some embodiments, the linker is selected from the group consisting of a cleavable linker and a non-cleavable linker. In some embodiments, the linker is a cleavable peptide linker. In some embodiments, the cleavable peptide linker has a formula: -MC-vc-PAB-. In some embodiments, the linker is a non-cleavable linker having a formula: -MC-.

In some embodiments, the subject has been previously treated for the cancer. In some embodiments, the subject did not respond to treatment or relapsed after first-line treatment. In some embodiments, the subject has not previously been treated for the cancer.

In some embodiments, the cancer is a lymphoma. In some embodiments, the lymphoma is a T-cell lymphoma. In some embodiments, the lymphoma is a B-cell lymphoma.

In some embodiments, the lymphoma is a non-Hodgkin lymphoma. In some embodiments, the subject has been previously treated for the non-Hodgkin lymphoma and the subject did not respond to treatment or relapsed after first-line treatment. In some embodiments, the subject has not been previously treated for the non-Hodgkin lymphoma. In some embodiments, the non-Hodgkin lymphoma is a mature T-cell lymphoma. In some embodiments, the non-Hodgkin lymphoma is diffuse large B-cell lymphoma (DLBCL), peripheral T-cell lymphoma (PTCL), anaplastic large cell lymphoma (ALCL) or cutaneous T-cell lymphoma (CTCL). In some embodiments, the non-Hodgkin lymphoma is cutaneous T-cell lymphoma (CTCL). In some embodiments, the cutaneous T-cell lymphoma (CTCL) is a mycosis fungoides (MF). In some embodiments, the mycosis fungoides is a CD30-positive mycosis fungoides (MF). In some embodiments, the cutaneous T-cell lymphoma (CTCL) is a primary cutaneous anaplastic large cell lymphoma (pcALCL). In some embodiments, the subject has received prior systemic treatment. In some embodiments, the non-Hodgkin lymphoma is anaplastic large cell lymphoma (ALCL). In some embodiments, the anaplastic large cell lymphoma (ALCL) is a systemic anaplastic large cell lymphoma (sALCL).

In some embodiments, the lymphoma is a Hodgkin lymphoma. In some embodiments, the subject has been previously treated for the Hodgkin lymphoma and the subject did not respond to treatment or relapsed after first-line treatment. In some embodiments, the subject relapsed after autologous stem cell transplant. In some embodiments, the subject relapsed after first-line treatment and the subject is ineligible for autologous stem cell transplant. In some embodiments, the subject has not been previously treated for the Hodgkin lymphoma. In some embodiments, the Hodgkin lymphoma is classical Hodgkin lymphoma (cHL). In some embodiments, the classical Hodgkin lymphoma (cHL) is advanced cHL. In some embodiments, the subject has been previously treated for cHL. In some embodiments, the subject has not been previously treated for cHL.

In some embodiments, the method further comprises administering one or more additional therapeutic agents capable of modulating the immune response. In some embodiments, the one or more additional therapeutic agents is not an antibody or antigen-binding fragment thereof. In some embodiments, the one or more additional therapeutic agents is an antibody or antigen-binding fragment thereof.

In some embodiments, the method further comprises administering one or more additional therapeutic agents. In some embodiments, the one or more additional therapeutic agents is a chemotherapy regimen consisting essentially of doxorubicin, vinblastine, and dacarbazine (AVD). In some embodiments, the one or more additional therapeutic agents is a chemotherapy regimen consisting essentially of Cyclophosphamide, Doxorubicin, and Prednisone (CHP). In some embodiments, the one or more additional therapeutic agents is an alkylating agent, an anthracycline, an antibiotic, an antifolate, an antimetabolite, an antitubulin agent, an auristatin, a chemotherapy sensitizer, a DNA minor groove binder, a DNA replication inhibitor, a duocarmycin, an etoposide, a fluorinated pyrimidine, a lexitropsin, a nitrosourea, a platinol, a purine antimetabolite, a puromycin, a radiation sensitizer, a steroid, a taxane, a topoisomerase inhibitor, and/or a vinca alkaloid. In some embodiments, the one or more additional therapeutic agents is selected from the group consisting of adriamycin, an androgen, anthramycin (AMC), asparaginase, 5-azacytidine, azathioprine, bleomycin, busulfan, buthionine sulfoximine, camptothecin, carboplatin, carmustine (BSNU), CC-1065, chlorambucil, cisplatin, colchicine, cyclophosphamide, cytarabine, cytidine arabinoside, cytochalasin B, dacarbazine, dactinomycin (formerly actinomycin), daunorubicin, decarbazine, docetaxel, doxorubicin, an estrogen, 5-fluordeoxyuridine, 5-fluorouracil, gramicidin D, hydroxydaunorubicin, hydroxyurea, idarubicin, ifosfamide, irinotecan, lomustine (CCNU), mechlorethamine, melphalan, 6-mercaptopurine, methotrexate, mithramycin, mitomycin C, mitoxantrone, nitroimidazole, paclitaxel, plicamycin, prednisone, prednisolone, procarbizine, streptozotocin, tenoposide, 6-thioguanine, thioTEPA, topotecan, vinblastine, vincristine, vinorelbine, VP-16 and VM-26. In some embodiments, the one or more additional therapeutic agents is an antibody or antigen-binding fragment thereof.

In some embodiments, the subject has cHL that has not been previously treated and the one or more additional therapeutic agents are adriamycin, dacarabazine and vinblastine. In some embodiments, the cHL is advanced cHL.

In some embodiments, the subject has a mature T-cell lymphoma that has not been previously treated and the one or more additional therapeutic agents are cyclophosphamide, hydroxydaunorubicin and prednisone.

In some embodiments, the subject has a mature T-cell lymphoma that has not been previously treated and the one or more additional therapeutic agents are cyclophosphamide, hydroxydaunorubicin and prednisolone.

In some embodiments, the method further comprises treating the subject with irradiation.

C. Methods of Treatment

In some embodiments, the present disclosure is directed to a method for treating a tumor or a subject afflicted with a tumor comprising administering to the subject a therapeutically effective amount of an antibody-drug conjugate, wherein the antibody-drug conjugate comprises an anti-CD30 antibody or an antigen-binding fragment thereof conjugated to a monomethyl auristatin (“anti-CD30 antibody-drug conjugate”). In some embodiments, the method of treating cancer in a subject comprises administering to the subject an antibody-drug conjugate, wherein the activity of CD30⁺ T regulatory (Treg) is decreased following administration of the antibody-drug conjugate. In some embodiments, the method of treating cancer in a subject comprises administering to the subject an antibody-drug conjugate, wherein the antibody-drug conjugate comprises an anti-CD30 antibody or an antigen-binding portion thereof conjugated to a monomethyl auristatin, wherein the ratio of CD8⁺ T cells to CD30⁺ T regulatory (Treg) cells in the subject is increased following administration of the antibody drug conjugate. In some embodiments, the method of treating cancer in a subject comprises administering to the subject an antibody-drug conjugate, wherein the antibody-drug conjugate comprises an anti-CD30 antibody or an antigen-binding portion thereof conjugated to a monomethyl auristatin, wherein the immune response is modulated following administration of the antibody-drug conjugate, wherein the modulation comprises increasing the ratio of CD8⁺ T cells to CD30⁺ T regulatory (Treg) cells in the subject.

In some embodiments, the tumor is derived from a Hodgkin lymphoma (HL), a non-Hodgkin lymphoma (NHL), or a combination thereof. In certain embodiments, the subject has received one, two, three, four, five or more prior cancer treatments. In other embodiments, the subject is treatment-naïve. In some embodiments, the subject has progressed on other cancer treatments. In some embodiments, the subject has received a previous cancer treatment and either did not respond or relapsed after the previous treatment. In some embodiments, the subject relapsed after previous cancer treatment and is ineligible for autologous stem cell transplant. In some embodiments, the subject relapsed after autologous stem cell transplant. In some embodiments, the tumor has reoccurred. In some embodiments, the tumor is metastatic. In other embodiments, the tumor is not metastatic.

In certain embodiments, the tumor is derived from an HL (e.g., a tumor comprising an HL). In certain embodiments, the subject has not been previously treated for the HL. In certain embodiments, the subject has been previously treated for the Hodgkin lymphoma and the subject did not respond to treatment or relapsed after first-line treatment. In certain embodiments, the subject relapsed after first-line treatment and the subject is ineligible for autologous stem cell transplant. In certain embodiments, the subject relapsed after autologous stem cell transplant. In certain embodiments, the HL is a classical HL (cHL; e.g., a nodular sclerosing HL, a mixed cellularity HL, a lymphocyte rich HL, or a lymphocyte depleted HL). In other embodiments, the HL is a nodular lymphocyte predominant type HL. In certain embodiments, the subject has not been previously treated for the cHL. In certain embodiments, the subject has not been previously treated for the cHL. In certain embodiments, the cHL is advanced cHL. In certain embodiments, the subject has not been previously treated for the advanced cHL. In certain embodiments, the subject has not been previously treated for the advanced cHL.

In other embodiments, the tumor is derived from a NHL. In some embodiments, the tumor comprises an NHL. In certain embodiments, the NHL is a relapsed or refractory NHL. In certain embodiments, the NHL has not been previously treated. In certain embodiments, the subject has not been previously treated for the NHL. In certain embodiments, the subject has been previously treated for the NHL and the subject did not respond to treatment or relapsed after first-line treatment. In some embodiments, the NHL is a B-cell lymphoma, e.g., a diffuse large B-cell lymphoma (DLBCL), a follicular lymphoma (FL), a Burkitt lymphoma, an immunoblastic large cell lymphoma, a precursor B-lymphoblastic lymphoma, a mantle cell lymphoma, or any combination thereof. In some embodiments, the NHL is a T-cell lymphoma, e.g., a cutaneous T-cell lymphoma (CTCL), a peripheral T-cell lymphoma (PTCL), a mycosis fungoides, an anaplastic large cell lymphoma, a precursor T-lymphoblastic lymphoma, or any combination thereof. In certain embodiments, the NHL is selected from a DLBCL, a PTCL, a CTCL, and any combination thereof. In certain embodiments, the NHL is a CTCL that is a relapsed or refractory CTCL. In certain embodiments, the T-cell lymphoma is a mature T-cell lymphoma. In certain embodiments, the subject has not been previously treated for the mature T-cell lymphoma.

In some embodiments, the method of treating cancer in a subject comprises administering to the subject an antibody drug conjugate, wherein the antibody-drug conjugate comprises an anti-CD30 antibody or an antigen-binding portion thereof conjugated to a monomethyl auristatin, wherein the activity of CD30⁺ T regulatory (Treg) is decreased following administration of the antibody-drug conjugate. In some embodiments, decreasing the activity of CD30⁺ Treg cells comprises a decrease in the number of CD30⁺ Treg cells. In some embodiments, the number of CD30⁺ Treg cells is decreased relative to one or more other types of CD4⁺ T cells. In some embodiments, the one or more other types of CD4⁺ T cells comprise Th1 cells, Th2 cells or Th17 cells. In some embodiments, the one or more other types of CD4⁺ T cells comprise Th1 CD30⁺ cells, Th2 CD30⁺ cells or Th17 CD30⁺ cells. In some embodiments, the number of CD30⁺ Treg cells is decreased relative to the number of CD30⁺ Treg cells in the subject prior to administration of the antibody-drug conjugate. In some embodiments, the number of CD30⁺ Treg cells is decreased relative to a subject who has not been treated with the antibody-drug conjugate. In some embodiments, decreasing the activity of CD30⁺ Treg cells comprises a decrease in the function of CD30⁺ Treg cells. In some embodiments, the decrease in the function of CD30⁺ Treg cells is relative to the function of CD30⁺ Treg cells in a subject prior to administration of the antibody-drug conjugate. In some embodiments, the decrease in the function of CD30⁺ Treg cells is relative to the function of CD30⁺ Treg cells in a subject who has not been treated with the antibody-drug conjugate.

In some embodiments, the CD30⁺ Treg cells are CD30⁺ inducible T regulatory (iTreg) cells or CD30⁺ peripheral T regulatory (pTreg) cells.

In some embodiments, the monomethyl auristatin is monomethyl auristatin E (MMAE). In some embodiments, the monomethyl auristatin is monomethyl auristatin F (MMAF).

In some embodiments, the anti-CD30 antibody is monoclonal anti-CD30 antibody AC10. In some embodiments, the anti-CD30 antibody is cAC10. In some embodiments, the antibody-drug conjugate is brentuximab vedotin.

In some embodiments, the anti-CD30 antibody comprises a heavy chain variable region and a light chain variable region, wherein the heavy chain variable region comprises:
(i) a CDR-H1 comprising the amino acid sequence of SEQ ID NO: 1;
(ii) a CDR-H2 comprising the amino acid sequence of SEQ ID NO: 2; and
(iii) a CDR-H3 comprising the amino acid sequence of SEQ ID NO: 3; and
   wherein the light chain variable region comprises:
(i) a CDR-L1 comprising the amino acid sequence of SEQ ID NO: 4;
(ii) a CDR-L2 comprising the amino acid sequence of SEQ ID NO: 5; and
(iii) a CDR-L3 comprising the amino acid sequence of SEQ ID NO: 6.

In some embodiments, the anti-CD30 antibody comprises a heavy chain variable region comprising the amino acid sequence of SEQ ID NO: 7 and a light chain variable region comprising the amino acid sequence of SEQ ID NO: 8.

In some embodiments, the antibody-drug conjugate further comprises a linker between the anti-CD30 antibody or antigen-binding portion thereof and the monomethyl auristatin. In some embodiments, the linker is selected from the group consisting of a cleavable linker and a non-cleavable linker. In some embodiments, the linker is a cleavable peptide linker. In some embodiments, the cleavable peptide linker has a formula: -MC-vc-PAB-. In some embodiments, the linker is a non-cleavable linker having a formula: -MC-.

In some embodiments, the subject has been previously treated for the cancer. In some embodiments, the subject did not respond to treatment or relapsed after first-line treatment. In some embodiments, the subject has not previously been treated for the cancer.

In some embodiments, the cancer is a lymphoma. In some embodiments, the lymphoma is a T-cell lymphoma. In some embodiments, the lymphoma is a B-cell lymphoma.

In some embodiments, the lymphoma is a non-Hodgkin lymphoma. In some embodiments, the subject has been previously treated for the non-Hodgkin lymphoma and the subject did not respond to treatment or relapsed after first-line treatment. In some embodiments, the subject has not been previously treated for the non-Hodgkin lymphoma. In some embodiments, the non-Hodgkin lymphoma is a mature T-cell lymphoma. In some embodiments, the non-Hodgkin lymphoma is diffuse large B-cell lymphoma (DLBCL), peripheral T-cell lymphoma (PTCL), anaplastic large cell lymphoma (ALCL) or cutaneous T-cell lymphoma (CTCL). In some embodiments, the non-Hodgkin lymphoma is cutaneous T-cell lymphoma (CTCL). In some embodiments, the cutaneous T-cell lymphoma (CTCL) is a mycosis fungoides (MF). In some embodiments, the mycosis fungoides is a CD30-positive mycosis fungoides (MF). In some embodiments, the cutaneous T-cell lymphoma (CTCL) is a primary cutaneous anaplastic large cell lymphoma (pcALCL). In some embodiments, the subject has received prior systemic treatment. In some embodiments, the non-Hodgkin lymphoma is anaplastic large cell lymphoma (ALCL). In some embodiments, the anaplastic large cell lymphoma (ALCL) is a systemic anaplastic large cell lymphoma (sALCL)

In some embodiments, the lymphoma is a Hodgkin lymphoma. In some embodiments, the subject has been previously treated for the Hodgkin lymphoma and the subject did not respond to treatment or relapsed after first-line treatment. In some embodiments, the subject relapsed after autologous stem cell transplant. In some embodiments, the subject relapsed after first-line treatment and the subject is ineligible for autologous stem cell transplant. In some embodiments, the subject has not been previously treated for the Hodgkin lymphoma. In some embodiments, the Hodgkin lymphoma is classical Hodgkin lymphoma (cHL). In some embodiments, the classical Hodgkin lymphoma (cHL) is advanced cHL. In some embodiments, the subject has been previously treated for cHL. In some embodiments, the subject has not been previously treated for cHL.

In some embodiments, the method further comprises administering one or more additional therapeutic agents capable of modulating the immune response. In some embodiments, the one or more additional therapeutic agents is not an antibody or antigen-binding fragment thereof. In some embodiments, the one or more additional therapeutic agents is an antibody or antigen-binding fragment thereof.

In some embodiments, the method further comprises administering one or more additional therapeutic agents. In some embodiments, the one or more additional therapeutic agents is a chemotherapy regimen consisting essentially of doxorubicin, vinblastine, and dacarbazine (AVD). In some embodiments, the one or more additional therapeutic agents is a chemotherapy regimen consisting essentially of Cyclophosphamide, Doxorubicin, and Prednisone (CHP). In some embodiments, the one or more additional therapeutic agents is an alkylating agent, an anthracycline, an antibiotic, an antifolate, an antimetabolite, an antitubulin agent, an auristatin, a chemotherapy sensitizer, a DNA minor groove binder, a DNA replication inhibitor, a duocarmycin, an etoposide, a fluorinated pyrimidine, a lexitropsin, a nitrosourea, a platinol, a purine antimetabolite, a puromycin, a radiation sensitizer, a steroid, a taxane, a topoisomerase inhibitor, and/or a vinca alkaloid. In some embodiments, the one or more additional therapeutic agents is selected from the group consisting of adriamycin, an androgen, anthramycin (AMC), asparaginase, 5-azacytidine, azathioprine, bleomycin, busulfan, buthionine sulfoximine, camptothecin, carboplatin, carmustine (BSNU), CC-1065, chlorambucil, cisplatin, colchicine, cyclophosphamide, cytarabine, cytidine arabinoside, cytochalasin B, dacarbazine, dactinomycin (formerly actinomycin), daunorubicin, decarbazine, docetaxel, doxorubicin, an estrogen, 5-fluordeoxyuridine, 5-fluorouracil, gramicidin D, hydroxydaunorubicin, hydroxyurea, idarubicin, ifosfamide, irinotecan, lomustine (CCNU), mechlorethamine, melphalan, 6-mercaptopurine, methotrexate, mithramycin, mitomycin C, mitoxantrone, nitroimidazole, paclitaxel, plicamycin, prednisone, prednisolone, procarbizine, streptozotocin, tenoposide, 6-thioguanine, thioTEPA, topotecan, vinblastine, vincristine, vinorelbine, VP-16 and VM-26. In some embodiments, the one or more additional therapeutic agents is an antibody or antigen-binding fragment thereof.

In some embodiments, the subject has cHL that has not been previously treated and wherein the one or more additional therapeutic agents are adriamycin, dacarabazine and vinblastine. In some embodiments, the cHL is advanced cHL.

In some embodiments, the subject has a mature T-cell lymphoma that has not been previously treated and wherein the one or more additional therapeutic agents are cyclophosphamide, hydroxydaunorubicin and prednisone.

In some embodiments, the subject has a mature T-cell lymphoma that has not been previously treated and wherein the one or more additional therapeutic agents are cyclophosphamide, hydroxydaunorubicin and prednisolone.

In some embodiments, the method further comprises treating the subject with irradiation.

In some embodiments, the method of treating cancer in a subject comprises administering to the subject an antibody-drug conjugate, wherein the antibody-drug conjugate comprises an anti-CD30 antibody or an antigen-binding portion thereof conjugated to a monomethyl auristatin, wherein the ratio of CD8⁺ T cells to CD30⁺ T regulatory (Treg) cells in the subject is increased following administration of the antibody drug conjugate. In some embodiments, the ratio of CD8⁺ T cells to CD30⁺ Treg cells is increased relative to the ratio of CD8⁺ T cells to CD30⁺ Treg cells in the subject prior to the administration of the antibody-drug conjugate. In some embodiments, the ratio of CD8⁺ T cells to CD30⁺ Treg cells is increased relative to the ratio of CD8⁺ T cells to CD30⁺ Treg cells in a subject who has not been treated with the antibody-drug conjugate.

In some embodiments, the CD30⁺ Treg cells are CD30⁺ inducible T regulatory (iTreg) cells or CD30⁺ peripheral T regulatory (pTreg) cells.

In some embodiments, the monomethyl auristatin is monomethyl auristatin E (MMAE). In some embodiments, the monomethyl auristatin is monomethyl auristatin F (MMAF).

In some embodiments, the anti-CD30 antibody is monoclonal anti-CD30 antibody AC10. In some embodiments, the anti-CD30 antibody is cAC10. In some embodiments, the antibody-drug conjugate is brentuximab vedotin.

In some embodiments, the anti-CD30 antibody comprises a heavy chain variable region and a light chain variable region, wherein the heavy chain variable region comprises:
(i) a CDR-H1 comprising the amino acid sequence of SEQ ID NO: 1;
(ii) a CDR-H2 comprising the amino acid sequence of SEQ ID NO: 2; and
(iii) a CDR-H3 comprising the amino acid sequence of SEQ ID NO: 3; and
   wherein the light chain variable region comprises:
(i) a CDR-L1 comprising the amino acid sequence of SEQ ID NO: 4;
(ii) a CDR-L2 comprising the amino acid sequence of SEQ ID NO: 5; and
(iii) a CDR-L3 comprising the amino acid sequence of SEQ ID NO: 6.

In some embodiments, the anti-CD30 antibody comprises a heavy chain variable region comprising the amino acid sequence of SEQ ID NO: 7 and a light chain variable region comprising the amino acid sequence of SEQ ID NO: 8.

In some embodiments, the antibody-drug conjugate further comprises a linker between the anti-CD30 antibody or antigen-binding portion thereof and the monomethyl auristatin. In some embodiments, the linker is selected from the group consisting of a cleavable linker and a non-cleavable linker. In some embodiments, the linker is a cleavable peptide linker. In some embodiments, the cleavable peptide linker has a formula: -MC-vc-PAB-. In some embodiments, the linker is a non-cleavable linker having a formula: -MC-.

In some embodiments, the subject has been previously treated for the cancer. In some embodiments, the subject did not respond to treatment or relapsed after first-line treatment. In some embodiments, the subject has not previously been treated for the cancer.

In some embodiments, the cancer is a lymphoma. In some embodiments, the lymphoma is a T-cell lymphoma. In some embodiments, the lymphoma is a B-cell lymphoma.

In some embodiments, the lymphoma is a non-Hodgkin lymphoma. In some embodiments, the subject has been previously treated for the non-Hodgkin lymphoma and the subject did not respond to treatment or relapsed after first-line treatment. In some embodiments, the subject has not been previously treated for the non-Hodgkin lymphoma. In some embodiments, the non-Hodgkin lymphoma is a mature T-cell lymphoma. In some embodiments, the non-Hodgkin lymphoma is diffuse large B-cell lymphoma (DLBCL), peripheral T-cell lymphoma (PTCL), anaplastic large cell lymphoma (ALCL) or cutaneous T-cell lymphoma (CTCL). In some embodiments, the non-Hodgkin lymphoma is cutaneous T-cell lymphoma (CTCL). In some embodiments, the cutaneous T-cell lymphoma (CTCL) is a mycosis fungoides (MF). In some embodiments, the mycosis fungoides is a CD30-positive mycosis fungoides (MF). In some embodiments, the cutaneous T-cell lymphoma (CTCL) is a primary cutaneous anaplastic large cell lymphoma (pcALCL). In some embodiments, the subject has received prior systemic treatment. In some embodiments, the non-Hodgkin lymphoma is anaplastic large cell lymphoma (ALCL). In some embodiments, the anaplastic large cell lymphoma (ALCL) is a systemic anaplastic large cell lymphoma (sALCL)

In some embodiments, the lymphoma is a Hodgkin lymphoma. In some embodiments, the subject has been previously treated for the Hodgkin lymphoma and the subject did not respond to treatment or relapsed after first-line treatment. In some embodiments, the subject relapsed after autologous stem cell transplant. In some embodiments, the subject relapsed after first-line treatment and the subject is ineligible for autologous stem cell transplant. In some embodiments, the subject has not been previously treated for the Hodgkin lymphoma. In some embodiments, the Hodgkin lymphoma is classical Hodgkin lymphoma (cHL). In some embodiments, the classical Hodgkin lymphoma (cHL) is advanced cHL. In some embodiments, the subject has been previously treated for cHL. In some embodiments, the subject has not been previously treated for cHL.

In some embodiments, the method further comprises administering one or more additional therapeutic agents capable of modulating the immune response. In some embodiments, the one or more additional therapeutic agents is not an antibody or antigen-binding fragment thereof. In some embodiments, the one or more additional therapeutic agents is an antibody or antigen-binding fragment thereof.

In some embodiments, the method further comprises administering one or more additional therapeutic agents. In some embodiments, the one or more additional therapeutic agents is a chemotherapy regimen consisting essentially of doxorubicin, vinblastine, and dacarbazine (AVD). In some embodiments, the one or more additional therapeutic agents is a chemotherapy regimen consisting essentially of Cyclophosphamide, Doxorubicin, and Prednisone (CHP). In some embodiments, the one or more additional therapeutic agents is an alkylating agent, an anthracycline, an antibiotic, an antifolate, an antimetabolite, an antitubulin agent, an auristatin, a chemotherapy sensitizer, a DNA minor groove binder, a DNA replication inhibitor, a duocarmycin, an etoposide, a fluorinated pyrimidine, a lexitropsin, a nitrosourea, a platinol, a purine antimetabolite, a puromycin, a radiation sensitizer, a steroid, a taxane, a topoisomerase inhibitor, and/or a vinca alkaloid. In some embodiments, the one or more additional therapeutic agents is selected from the group consisting of adriamycin, an androgen, anthramycin (AMC), asparaginase, 5-azacytidine, azathioprine, bleomycin, busulfan, buthionine sulfoximine, camptothecin, carboplatin, carmustine (BSNU), CC-1065, chlorambucil, cisplatin, colchicine, cyclophosphamide, cytarabine, cytidine arabinoside, cytochalasin B, dacarbazine, dactinomycin (formerly actinomycin), daunorubicin, decarbazine, docetaxel, doxorubicin, an estrogen, 5-fluordeoxyuridine, 5-fluorouracil, gramicidin D, hydroxydaunorubicin, hydroxyurea, idarubicin, ifosfamide, irinotecan, lomustine (CCNU), mechlorethamine, melphalan, 6-mercaptopurine, methotrexate, mithramycin, mitomycin C, mitoxantrone, nitroimidazole, paclitaxel, plicamycin, prednisone, prednisolone, procarbizine, streptozotocin, tenoposide, 6-thioguanine, thioTEPA, topotecan, vinblastine, vincristine, vinorelbine, VP-16 and VM-26. In some embodiments, the one or more additional therapeutic agents is an antibody or antigen-binding fragment thereof.

In some embodiments, the subject has cHL that has not been previously treated and wherein the one or more additional therapeutic agents are adriamycin, dacarabazine and vinblastine. In some embodiments, the cHL is advanced cHL.

In some embodiments, the subject has a mature T-cell lymphoma that has not been previously treated and wherein the one or more additional therapeutic agents are cyclophosphamide, hydroxydaunorubicin and prednisone.

In some embodiments, the subject has a mature T-cell lymphoma that has not been previously treated and wherein the one or more additional therapeutic agents are cyclophosphamide, hydroxydaunorubicin and prednisolone.

In some embodiments, the method further comprises treating the subject with irradiation.

In some embodiments, the method of treating cancer in a subject comprising administering to the subject an antibody-drug conjugate, wherein the antibody-drug conjugate comprises an anti-CD30 antibody or an antigen-binding portion thereof conjugated to a monomethyl auristatin, wherein the immune response is modulated following administration of the antibody-drug conjugate, wherein the modulation comprises increasing the ratio of CD8⁺ T cells to CD30⁺ T regulatory (Treg) cells in the subject. In some embodiments, the ratio of CD8⁺ T cells to CD30⁺ Treg cells is increased relative to the ratio of CD8⁺ T cells to CD30⁺ Treg cells in the subject prior to the administration of the antibody-drug conjugate. In some embodiments, the ratio of CD8⁺ T cells to CD30⁺ Treg cells is increased relative to the ratio of CD8⁺ T cells to CD30⁺ Treg cells in a subject who has not been treated with the antibody-drug conjugate.

In some embodiments, the CD30⁺ Treg cells are CD30⁺ inducible T regulatory (iTreg) cells or CD30⁺ peripheral T regulatory (pTreg) cells.

In some embodiments, the monomethyl auristatin is monomethyl auristatin E (MMAE). In some embodiments, the monomethyl auristatin is monomethyl auristatin F (MMAF).

In some embodiments, the anti-CD30 antibody is monoclonal anti-CD30 antibody AC10. In some embodiments, the anti-CD30 antibody is cAC10. In some embodiments, the antibody-drug conjugate is brentuximab vedotin.

In some embodiments, the anti-CD30 antibody comprises a heavy chain variable region and a light chain variable region, wherein the heavy chain variable region comprises:
(i) a CDR-H1 comprising the amino acid sequence of SEQ ID NO: 1;
(ii) a CDR-H2 comprising the amino acid sequence of SEQ ID NO: 2; and
(iii) a CDR-H3 comprising the amino acid sequence of SEQ ID NO: 3; and
   wherein the light chain variable region comprises:
(i) a CDR-L1 comprising the amino acid sequence of SEQ ID NO: 4;
(ii) a CDR-L2 comprising the amino acid sequence of SEQ ID NO: 5; and
(iii) a CDR-L3 comprising the amino acid sequence of SEQ ID NO: 6.

In some embodiments, the anti-CD30 antibody comprises a heavy chain variable region comprising the amino acid sequence of SEQ ID NO: 7 and a light chain variable region comprising the amino acid sequence of SEQ ID NO: 8.

In some embodiments, the antibody-drug conjugate further comprises a linker between the anti-CD30 antibody or antigen-binding portion thereof and the monomethyl auristatin. In some embodiments, the linker is selected from the group consisting of a cleavable linker and a non-cleavable linker. In some embodiments, the linker is a cleavable peptide linker. In some embodiments, the cleavable peptide linker has a formula: -MC-vc-PAB-. In some embodiments, the linker is a non-cleavable linker having a formula: -MC-.

In some embodiments, the subject has been previously treated for the cancer. In some embodiments, the subject did not respond to treatment or relapsed after first-line treatment. In some embodiments, the subject has not previously been treated for the cancer.

In some embodiments, the cancer is a lymphoma. In some embodiments, the lymphoma is a T-cell lymphoma. In some embodiments, the lymphoma is a B-cell lymphoma.

In some embodiments, the lymphoma is a non-Hodgkin lymphoma. In some embodiments, the subject has been previously treated for the non-Hodgkin lymphoma and the subject did not respond to treatment or relapsed after first-line treatment. In some embodiments, the subject has not been previously treated for the non-Hodgkin lymphoma. In some embodiments, the non-Hodgkin lymphoma is a mature T-cell lymphoma. In some embodiments, the non-Hodgkin lymphoma is diffuse large B-cell lymphoma (DLBCL), peripheral T-cell lymphoma (PTCL), anaplastic large cell lymphoma (ALCL) or cutaneous T-cell lymphoma (CTCL). In some embodiments, the non-Hodgkin lymphoma is cutaneous T-cell lymphoma (CTCL). In some embodiments, the cutaneous T-cell lymphoma (CTCL) is a mycosis fungoides (MF). In some embodiments, the mycosis fungoides is a CD30-positive mycosis fungoides (MF). In some embodiments, the cutaneous T-cell lymphoma (CTCL) is a primary cutaneous anaplastic large cell lymphoma (pcALCL). In some embodiments, the subject has received prior systemic treatment. In some embodiments, the non-Hodgkin lymphoma is anaplastic large cell lymphoma (ALCL). In some embodiments, the anaplastic large cell lymphoma (ALCL) is a systemic anaplastic large cell lymphoma (sALCL).

In some embodiments, the lymphoma is a Hodgkin lymphoma. In some embodiments, the subject has been previously treated for the Hodgkin lymphoma and the subject did not respond to treatment or relapsed after first-line treatment. In some embodiments, the subject relapsed after autologous stem cell transplant. In some embodiments, the subject relapsed after first-line treatment and the subject is ineligible for autologous stem cell transplant. In some embodiments, the subject has not been previously treated for the Hodgkin lymphoma. In some embodiments, the Hodgkin lymphoma is classical Hodgkin lymphoma (cHL). In some embodiments, the classical Hodgkin lymphoma (cHL) is advanced cHL. In some embodiments, the subject has been previously treated for cHL. In some embodiments, the subject has not been previously treated for cHL.

In some embodiments, the method further comprises administering one or more additional therapeutic agents capable of modulating the immune response. In some embodiments, the one or more additional therapeutic agents is not an antibody or antigen-binding fragment thereof. In some embodiments, the one or more additional therapeutic agents is an antibody or antigen-binding fragment thereof.

In some embodiments, the method further comprises administering one or more additional therapeutic agents. In some embodiments, the one or more additional therapeutic agents is a chemotherapy regimen consisting essentially of doxorubicin, vinblastine, and dacarbazine (AVD). In some embodiments, the one or more additional therapeutic agents is a chemotherapy regimen consisting essentially of Cyclophosphamide, Doxorubicin, and Prednisone (CHP). In some embodiments, the one or more additional therapeutic agents is an alkylating agent, an anthracycline, an antibiotic, an antifolate, an antimetabolite, an antitubulin agent, an auristatin, a chemotherapy sensitizer, a DNA minor groove binder, a DNA replication inhibitor, a duocarmycin, an etoposide, a fluorinated pyrimidine, a lexitropsin, a nitrosourea, a platinol, a purine antimetabolite, a puromycin, a radiation sensitizer, a steroid, a taxane, a topoisomerase inhibitor, and/or a vinca alkaloid. In some embodiments, the one or more additional therapeutic agents is selected from the group consisting of adriamycin, an androgen, anthramycin (AMC), asparaginase, 5-azacytidine, azathioprine, bleomycin, busulfan, buthionine sulfoximine, camptothecin, carboplatin, carmustine (BSNU), CC-1065, chlorambucil, cisplatin, colchicine, cyclophosphamide, cytarabine, cytidine arabinoside, cytochalasin B, dacarbazine, dactinomycin (formerly actinomycin), daunorubicin, decarbazine, docetaxel, doxorubicin, an estrogen, 5-fluordeoxyuridine, 5-fluorouracil, gramicidin D, hydroxydaunorubicin, hydroxyurea, idarubicin, ifosfamide, irinotecan, lomustine (CCNU), mechlorethamine, melphalan, 6-mercaptopurine, methotrexate, mithramycin, mitomycin C, mitoxantrone, nitroimidazole, paclitaxel, plicamycin, prednisone, prednisolone, procarbizine, streptozotocin, tenoposide, 6-thioguanine, thioTEPA, topotecan, vinblastine, vincristine, vinorelbine, VP-16 and VM-26. In some embodiments, the one or more additional therapeutic agents is an antibody or antigen-binding fragment thereof.

In some embodiments, the subject has cHL that has not been previously treated and wherein the one or more additional therapeutic agents are adriamycin, dacarabazine and vinblastine. In some embodiments, the cHL is advanced cHL.

In some embodiments, the subject has a mature T-cell lymphoma that has not been previously treated and wherein the one or more additional therapeutic agents are cyclophosphamide, hydroxydaunorubicin and prednisone.

In some embodiments, the subject has a mature T-cell lymphoma that has not been previously treated and wherein the one or more additional therapeutic agents are cyclophosphamide, hydroxydaunorubicin and prednisolone.

In some embodiments, the method further comprises treating the subject with irradiation.

In some embodiments, the method of treating cancer is a method of treating cutaneous T-cell lymphoma (CTCL) in a subject comprising administering to the subject an antibody drug conjugate, wherein the antibody drug conjugate comprises an anti-CD30 antibody or an antigen-binding portion thereof conjugated to a monomethyl auristatin. In some embodiments, the ratio of CD8⁺ T cells to CD30⁺ T regulatory (Treg) in the subject is increased following administration of the antibody drug conjugate. In some embodiments, the immune response is modulated following administration of the antibody drug conjugate, wherein the modulation comprises increasing the ratio of CD8⁺ T cells to CD30⁺ T regulatory (Treg). In some embodiments, the ratio of CD8⁺ T cells to CD30⁺ Treg cells is increased relative to the ratio of CD8⁺ T cells to CD30⁺ Treg cells in the subject prior to the administration of the antibody-drug conjugate. In some embodiments, the ratio of CD8⁺ T cells to CD30⁺ Treg cells is increased relative to the ratio of CD8⁺ T cells to CD30⁺ Treg cells in a subject who has not been treated with the antibody-drug conjugate. In some embodiments, the subject did not respond to treatment for the CTCL or relapsed after first-line treatment for the CTCL. In some embodiments, the subject has not been previously treated for the CTCL.

In some embodiments, the CD30⁺ Treg cells are CD30⁺ inducible T regulatory (iTreg) cells or CD30⁺ peripheral T regulatory (pTreg) cells.

In some embodiments, the monomethyl auristatin is monomethyl auristatin E (MMAE). In some embodiments, the monomethyl auristatin is monomethyl auristatin F (MMAF).

In some embodiments, the anti-CD30 antibody is monoclonal anti-CD30 antibody AC10. In some embodiments, the anti-CD30 antibody is cAC10. In some embodiments, the antibody-drug conjugate is brentuximab vedotin.

In some embodiments, the anti-CD30 antibody comprises a heavy chain variable region and a light chain variable region, wherein the heavy chain variable region comprises:
(i) a CDR-H1 comprising the amino acid sequence of SEQ ID NO: 1;
(ii) a CDR-H2 comprising the amino acid sequence of SEQ ID NO: 2; and
(iii) a CDR-H3 comprising the amino acid sequence of SEQ ID NO: 3; and
   wherein the light chain variable region comprises:
(i) a CDR-L1 comprising the amino acid sequence of SEQ ID NO: 4;
(ii) a CDR-L2 comprising the amino acid sequence of SEQ ID NO: 5; and
(iii) a CDR-L3 comprising the amino acid sequence of SEQ ID NO: 6.

In some embodiments, the anti-CD30 antibody comprises a heavy chain variable region comprising the amino acid sequence of SEQ ID NO: 7 and a light chain variable region comprising the amino acid sequence of SEQ ID NO: 8.

In some embodiments, the antibody-drug conjugate further comprises a linker between the anti-CD30 antibody or antigen-binding portion thereof and the monomethyl auristatin. In some embodiments, the linker is selected from the group consisting of a cleavable linker and a non-cleavable linker. In some embodiments, the linker is a cleavable peptide linker. In some embodiments, the cleavable peptide linker has a formula: -MC-vc-PAB-. In some embodiments, the linker is a non-cleavable linker having a formula: -MC-.

In some embodiments, the subject has been previously treated for the cancer. In some embodiments, the subject did not respond to treatment or relapsed after first-line treatment. In some embodiments, the subject has not previously been treated for the cancer.

In some embodiments, the method further comprises administering one or more additional therapeutic agents capable of modulating the immune response. In some embodiments, the one or more additional therapeutic agents is not an antibody or antigen-binding fragment thereof. In some embodiments, the one or more additional therapeutic agents is an antibody or antigen-binding fragment thereof.

In some embodiments, the method further comprises administering one or more additional therapeutic agents. In some embodiments, the one or more additional therapeutic agents is a chemotherapy regimen consisting essentially of doxorubicin, vinblastine, and dacarbazine (AVD). In some embodiments, the one or more additional therapeutic agents is a chemotherapy regimen consisting essentially of Cyclophosphamide, Doxorubicin, and Prednisone (CHP). In some embodiments, the one or more additional therapeutic agents is an alkylating agent, an anthracycline, an antibiotic, an antifolate, an antimetabolite, an antitubulin agent, an auristatin, a chemotherapy sensitizer, a DNA minor groove binder, a DNA replication inhibitor, a duocarmycin, an etoposide, a fluorinated pyrimidine, a lexitropsin, a nitrosourea, a platinol, a purine antimetabolite, a puromycin, a radiation sensitizer, a steroid, a taxane, a topoisomerase inhibitor, and/or a vinca alkaloid. In some embodiments, the one or more additional therapeutic agents is selected from the group consisting of adriamycin, an androgen, anthramycin (AMC), asparaginase, 5-azacytidine, azathioprine, bleomycin, busulfan, buthionine sulfoximine, camptothecin, carboplatin, carmustine (BSNU), CC-1065, chlorambucil, cisplatin, colchicine, cyclophosphamide, cytarabine, cytidine arabinoside, cytochalasin B, dacarbazine, dactinomycin (formerly actinomycin), daunorubicin, decarbazine, docetaxel, doxorubicin, an estrogen, 5-fluordeoxyuridine, 5-fluorouracil, gramicidin D, hydroxydaunorubicin, hydroxyurea, idarubicin, ifosfamide, irinotecan, lomustine (CCNU), mechlorethamine, melphalan, 6-mercaptopurine, methotrexate, mithramycin, mitomycin C, mitoxantrone, nitroimidazole, paclitaxel, plicamycin, prednisone, prednisolone, procarbizine, streptozotocin, tenoposide, 6-thioguanine, thioTEPA, topotecan, vinblastine, vincristine, vinorelbine, VP-16 and VM-26. In some embodiments, the one or more additional therapeutic agents is an antibody or antigen-binding fragment thereof.

In some embodiments, the method further comprises treating the subject with irradiation.

In some embodiments, the method of treating cancer is a method of treating a non-Hodgkin lymphoma or a Hodgkin lymphoma in a subject comprising administering to the subject an antibody drug conjugate, wherein the antibody drug conjugate comprises an anti-CD30 antibody or an antigen-binding portion thereof conjugated to a monomethyl auristatin. In some embodiments, the ratio of CD8⁺ T cells to CD30⁺ T regulatory (Treg) in the subject is increased following administration of the antibody drug conjugate. In some embodiments, the immune response is modulated following administration of the antibody drug conjugate, wherein the modulation comprises increasing the ratio of CD8⁺ T cells to CD30⁺ T regulatory (Treg). In some embodiments, the subject has not previously been treated for the non-Hodgkin lymphoma or Hodgkin lymphoma. In some embodiments, the cancer is a non-Hodgkin lymphoma. In some embodiments, the subject has not previously been treated for the non-Hodgkin lymphoma. In some embodiments, non-Hodgkin lymphoma is a mature T-cell lymphoma. In some embodiments the subject has not been previously been treated for the mature T-cell lymphoma and the method further comprises administering one or more additional therapeutic agents. In some embodiments, the one or more additional therapeutic agents comprise one or more agents selected from the group consisting of cyclophosphamide, hydroxydaunorubicin, prednisone and prednisolone. In some embodiments, the one or more additional therapeutic agents is a chemotherapy regimen consisting essentially of cyclophosphamide, hydroxydaunorubicin and prednisone. In some embodiments, the one or more additional therapeutic agents is a chemotherapy regimen consisting essentially of cyclophosphamide, hydroxydaunorubicin and prednisolone. In some embodiments, the cancer is a Hodgkin lymphoma. In some embodiments, the Hodgkin lymphoma is classical Hodgkin lymphoma. In some embodiments, the subject has not been previously treated for the classical Hodgkin lymphoma and the method further comprises administering one or more additional therapeutic agents. In some embodiments, the one or more additional therapeutic agents is a chemotherapy regimen consisting essentially of adriamycin, vinblastine and dacarbazine. In some embodiments, the one or more additional therapeutic agents is a chemotherapy regimen consisting essentially of adriamycin, vinblastine and dacarbazine.

In some embodiments, the present methods further comprise administering one or more additional therapeutic agents capable of modulating the immune response. In some embodiments, the one or more additional therapeutic agents is not an antibody or antigen-binding fragment thereof. In some embodiments, the one or more additional therapeutic agents is an antibody or antigen-binding fragment thereof.

In some embodiments, the present methods further comprise administering one or more additional therapeutic agents. In some embodiments, the one or more additional therapeutic agents is an alkylating agent, an anthracycline, an antibiotic, an antifolate, an antimetabolite, an antitubulin agent, an auristatin, a chemotherapy sensitizer, a DNA minor groove binder, a DNA replication inhibitor, a duocarmycin, an etoposide, a fluorinated pyrimidine, a lexitropsin, a nitrosourea, a platinol, a purine antimetabolite, a puromycin, a radiation sensitizer, a steroid, a taxane, a topoisomerase inhibitor, and/or a vinca alkaloid. In some embodiments, the one or more additional therapeutic agents is selected from the group consisting of adriamycin, an androgen, anthramycin (AMC), asparaginase, 5-azacytidine, azathioprine, bleomycin, busulfan, buthionine sulfoximine, camptothecin, carboplatin, carmustine (BSNi), CC-1065, chlorambucil, cisplatin, colchicine, cyclophosphanide, cytarabine, cytidine arabinoside, cytochalasin B, dacarbazine, dactinomycin (formerly actinomycin), daunorubicin, decarbazine, docetaxel, doxorubicin, an estrogen, 5-fluordeoxyuridine, 5-fluorouracil, gramicidin D, hydroxydaunorubicin, hydroxyurea, idarubicin, ifosfamide, irinotecan, lomustine (CCNU), mechlorethamine, melphalan, 6-mercaptopurine, methotrexate, mithramycin, mitomycin C, mitoxantrone, nitroimidazole, paclitaxel, plicamycin, prednisone, prednisolone, procarbizine, streptozotocin, tenoposide, 6-thioguanine, thioTEPA, topotecan, vinblastine, vincristine, vinorelbine, VP-16 and VM-26. In some embodiments, the one or more additional therapeutic agents are adriamycin, dacarabazine and vinblastine. In some embodiments, the one or more additional therapeutic agents are cyclophosphamide, hydroxydaunorubicin and prednisone. In some embodiments, the one or more additional therapeutic agents are cyclophosphamide, hydroxydaunorubicin and prednisolone.

In other embodiments, the present methods comprise administering an effective amount of an anti-CD30 antibody-drug conjugate. An effective amount of an anti-CD30 antibody-drug conjugate can be a flat dose or a weight based dose.

In certain embodiments, the therapy of the present disclosure (e.g., administration of an anti-CD30 antibody-drug conjugate) effectively increases the duration of survival of the subject. For example, the duration of survival of the subject is increased by at least about 1 month, at least about 2 months, at least about 3 months, at least about 4 months, at least about 5 months, at least about 6 months, at least about 7 months, at least about 8 months, at least about 9 months, at least about 10 months, at least about 11 months, or at least about 1 year or more when compared to another subject treated with another therapy.

In certain embodiments, the therapy of the present disclosure effectively increases the duration of progression-free survival of the subject. For example, the progression free survival of the subject is increased by at least about 1 month, at least about 2 months, at least about 3 months, at least about 4 months, at least about 5 months, at least about 6 months, at least about 7 months, at least about 8 months, at least about 9 months, at least about 10 months, at least about 11 months, or at least about 1 year when compared to another subject treated with another therapy

In certain embodiments, the therapy of the present disclosure effectively increases the response rate in a group of subjects. For example, the response rate in a group of subjects is increased by at least about 2%, at least about 3%, at least about 4%, at least about 5%, at least about 10%, at least about 15%, at least about 20%, at least about 25%, at least about 30%, at last about 35%, at least about 40%, at least about 45%, at least about 50%, at least about 55%, at least about 60%, at least about 70%, at least about 75%, at least about 80%, at least about 85%, at least about 90%, at least about 95%, at least about 99% or at least about 100% when compared to another group of subjects treated with another therapy.

### III. Compositions

In some aspects, provided herein are compositions (e.g., pharmaceutical compositions) comprising any of the anti-CD30 antibody-drug conjugates described herein (e.g., an anti-CD30 antibody drug conjugate that binds to human CD30). The anti-CD30 drug-conjugates of the present disclosure can be constituted in a composition, e.g., a pharmaceutical composition containing an antibody drug-conjugate and a pharmaceutically acceptable carrier. As used herein, a “pharmaceutically acceptable carrier” includes any and all solvents, dispersion media, coatings, antibacterial and antifungal agents, isotonic and absorption delaying agents, and the like that are physiologically compatible. In some embodiments, the carrier for a composition containing an antibody drug-conjugate is suitable for intravenous, intramuscular, subcutaneous, parenteral, spinal, or epidermal administration (e.g., by injection or infusion). A pharmaceutical composition of the disclosure can include one or more pharmaceutically acceptable salts, anti-oxidants, aqueous and non-aqueous carriers, and/or adjuvants such as preservatives, wetting agents, emulsifying agents, and dispersing agents.

Therapeutic formulations are prepared for storage by mixing the active ingredient having the desired degree of purity with optional pharmaceutically acceptable carriers, excipients or stabilizers (Remington: The Science and Practice of Pharmacy, 20th Ed., Lippincott Williams & Wiklins, Pub., Gennaro Ed., Philadelphia, Pa. 2000). Acceptable carriers, excipients, or stabilizers are nontoxic to recipients at the dosages and concentrations employed, and include buffers, antioxidants including ascorbic acid, methionine, Vitamin E, sodium metabisulfite; preservatives, isotonicifiers, stabilizers, metal complexes (e.g., Zn-protein complexes); chelating agents such as EDTA and/or non-ionic surfactants.

Buffers can be used to control the pH in a range which optimizes the therapeutic effectiveness, especially if stability is pH dependent. Buffers can be present at concentrations ranging from about 50 mM to about 250 mM. Suitable buffering agents for use with the present invention include both organic and inorganic acids and salts thereof. For example, citrate, phosphate, succinate, tartrate, fumarate, gluconate, oxalate, lactate, acetate. Additionally, buffers may be comprised of histidine and trimethylamine salts such as Tris.

Preservatives can be added to prevent microbial growth, and are typically present in a range from about 0.2%-1.0% (w/v). Suitable preservatives for use with the present invention include octadecyldimethylbenzyl ammonium chloride; hexamethonium chloride; benzalkonium halides (e.g., chloride, bromide, iodide), benzethonium chloride; thimerosal, phenol, butyl or benzyl alcohol; alkyl parabens such as methyl or propyl paraben; catechol; resorcinol; cyclohexanol, 3-pentanol, and m-cresol.

Tonicity agents, sometimes known as “stabilizers” can be present to adjust or maintain the tonicity of liquid in a composition. When used with large, charged biomolecules such as proteins and antibodies, they are often termed “stabilizers” because they can interact with the charged groups of the amino acid side chains, thereby lessening the potential for inter and intra-molecular interactions. Tonicity agents can be present in any amount between about 0.1% to about 25% by weight or between about 1 to about 5% by weight, taking into account the relative amounts of the other ingredients. In some embodiments, tonicity agents include polyhydric sugar alcohols, trihydric or higher sugar alcohols, such as glycerin, erythritol, arabitol, xylitol, sorbitol and mannitol.

Additional excipients include agents which can serve as one or more of the following: (1) bulking agents, (2) solubility enhancers, (3) stabilizers and (4) and agents preventing denaturation or adherence to the container wall. Such excipients include: polyhydric sugar alcohols (enumerated above); amino acids such as alanine, glycine, glutamine, asparagine, histidine, arginine, lysine, ornithine, leucine, 2-phenylalanine, glutamic acid, threonine, etc.; organic sugars or sugar alcohols such as sucrose, lactose, lactitol, trehalose, stachyose, mannose, sorbose, xylose, ribose, ribitol, myoinisitose, myoinisitol, galactose, galactitol, glycerol, cyclitols (e.g., inositol), polyethylene glycol; sulfur containing reducing agents, such as urea, glutathione, thioctic acid, sodium thioglycolate, thioglycerol, α-monothioglycerol and sodium thio sulfate; low molecular weight proteins such as human serum albumin, bovine serum albumin, gelatin or other immunoglobulins; hydrophilic polymers such as polyvinylpyrrolidone; monosaccharides (e.g., xylose, mannose, fructose, glucose; disaccharides (e.g., lactose, maltose, sucrose); trisaccharides such as raffinose; and polysaccharides such as dextrin or dextran.

Non-ionic surfactants or detergents (also known as “wetting agents”) can be present to help solubilize the therapeutic agent (e.g., anti-CD30 antibody drug-conjugate) as well as to protect the therapeutic protein (e.g., anti-CD30 antibody) against agitation-induced aggregation, which also permits the formulation to be exposed to shear surface stress without causing denaturation of the active therapeutic protein. Non-ionic surfactants are present in a range of about 0.05 mg/ml to about 1.0 mg/ml or about 0.07 mg/ml to about 0.2 mg/ml. In some embodiments, non-ionic surfactants are present in a range of about 0.001% to about 0.1% w/v or about 0.01% to about 0.1% w/v or about 0.01% to about 0.025% w/v.

Suitable non-ionic surfactants include polysorbates (20, 40, 60, 65, 80, etc.), polyoxamers (184, 188, etc.), PLURONIC® polyols, TRITON®, polyoxyethylene sorbitan monoethers (TWEEN®-20, TWEEN®-80, etc.), lauromacrogol 400, polyoxyl 40 stearate, polyoxyethylene hydrogenated castor oil 10, 50 and 60, glycerol monostearate, sucrose fatty acid ester, methyl celluose and carboxymethyl cellulose. Anionic detergents that can be used include sodium lauryl sulfate, dioctyle sodium sulfosuccinate and dioctyl sodium sulfonate. Cationic detergents include benzalkonium chloride or benzethonium chloride.

In order for the formulations to be used for in vivo administration, they must be sterile. The formulation may be rendered sterile by filtration through sterile filtration membranes. The therapeutic compositions herein generally are placed into a container having a sterile access port, for example, an intravenous solution bag or vial having a stopper pierceable by a hypodermic injection needle.

The route of administration is in accordance with known and accepted methods, such as by single or multiple bolus or infusion over a long period of time in a suitable manner, e.g., injection or infusion by subcutaneous, intravenous, intraperitoneal, intramuscular, intraarterial, intralesional or intraarticular routes, topical administration, inhalation or by sustained release or extended-release means.

The formulation herein may also contain more than one active compound as necessary for the particular indication being treated, preferably those with complementary activities that do not adversely affect each other. Alternatively, or in addition, the composition may comprise an alkylating agent, an anthracycline, an antibiotic, an antifolate, an antimetabolite, an antitubulin agent, an auristatin, a cheiotherapy sensitizer, a DNA minor groove binder, a DNA replication inhibitor, a duocarmycin, an etoposide, a fluorinated pyrimidine, a lexitropsin, a nitrosourea, a platinol, a purine antimetabolite, a puromycin, a radiation sensitizer, a steroid, a taxane, a topoisomerase inhibitor, and/or a vinca alkaloid. In some embodiments, the composition may comprise adriamycin, an androgen, anthramycin (AMC), asparaginase, 5-azacytidine, azathioprine, bleomycin, busulfan, buthionine sulfoximine, camptothecin, carboplatin, carmustine (BSNU), CC-1065, chlorambucil, cisplatin, colchicine, cyclophosphamide, cytarabine, cytidine arabinoside, cytochalasin B, dacarbazine, dactinomycin (formerly actinomycin), daunorubicin, decarbazine, docetaxel, doxorubicin, an estrogen, 5-fluordeoxyuridine, 5-fluorouracil, gramicidin D, hydroxydaunorubicin, hydroxyurea, idarubicin, ifosfamide, irinotecan, lomustine (CCNU), mechlorethamine, melphalan, 6-mercaptopurine, methotrexate, mithramycin, mitomycin C, mitoxantrone, nitroimidazole, paclitaxel, plicamycin, prednisone, prednisolone, procarbizine, streptozotocin, tenoposide, 6-thioguanine, thioTEPA, topotecan, vinblastine, vincristine, vinorelbine, VP-16 or VM-26. In some embodiments, the composition may comprise Cyclophosphamide, Doxorubicin, and Prednisone (CHP). In some embodiments, the composition may comprise cyclophosphamide, Doxorubicin and Prednisolone. In some embodiments, the composition may comprise doxorubicin, vinblastine, and dacarbazine (AVD). Such molecules are suitably present in combination in amounts that are effective for the purpose intended.

Dosage regimens are adjusted to provide the optimum desired response, e.g., a maximal therapeutic response and/or minimal adverse effects. In some embodiments, the anti-CD30 antibody drug-conjugate (e.g., brentuximab vedotin) is administered at a weight-based dose. For administration of an anti-CD30 antibody drug-conjugate (e.g., brentuximab vedotin), the dosage can range from about 0.01 mg/kg to about 20 mg/kg, about 0.05 mg/kg to about 20 mg/kg, about 0.1 mg/kg to about 20 mg/kg, about 0.1 mg/kg to about 15 mg/kg, about 0.1 mg/kg to about 10 mg/kg, about 0.1 mg/kg to about 5 mg/kg, about 0.1 mg/kg to about 4 mg/kg, about 0.1 mg/kg to about 3 mg/kg, about 0.1 to about 2 mg/kg, about 1 to about 10 mg/kg, about 1 to about 10 mg/kg, about 1 to about 8 mg/kg, about 1 to about 5 mg/kg, about 1 to about 3 mg/kg, about 1 to about 2 mg/kg of the subject's body weight. For example, dosages can be about 0.05 mg/kg, about 0.1 mg/kg, about 0.2 mg/kg, about 0.3 mg/kg, about 0.4 mg/kg, about 0.5 mg/kg, about 0.6 mg/kg, about 0.7 mg/kg, about 0.8 mg/kg, about 0.9 mg/kg, about 1.0 mg/kg, about 1.1 mg/kg, about 1.2 mg/kg, about 1.3 mg/kg, about 1.4 mg/kg, about 1.5 mg/kg, about 1.6 mg/kg, about 1.7 mg/kg, about 1.8 mg/kg, about 1.9 mg/kg, about 2.0 mg/kg, about 2.1 mg/kg, about 2.2 mg/kg, about 2.3 mg/kg, about 2.4 mg/kg, about 2.5 mg/kg, about 2.6 mg/kg, about 2.7 mg/kg, about 2.8 mg/kg, about 2.9 mg/kg, about 3 mg/kg, about 4 mg/kg, about 5 mg/kg, about 6 mg/kg, about 7 mg/kg, about 8 mg/kg, about 9 mg/kg, about 10 mg/kg, about 11 mg/kg, about 12 mg/kg, about 13 mg/kg, about 14 mg/kg, about 15 mg/kg, or about 20 mg/kg of the subject's body weight.

In some embodiments, the dosage of the anti-CD30 antibody drug-conjugate (e.g., brentuximab vedotin) is 0.1 mg/kg body weight. In other embodiments, the dosage of the anti-CD30 antibody drug-conjugate (e.g., brentuximab vedotin) is 0.2 mg/kg body weight. In other embodiments, the dosage of the anti-CD30 antibody drug-conjugate (e.g., brentuximab vedotin) is 0.3 mg/kg body weight. In other embodiments, the dosage of the anti-CD30 antibody drug-conjugate (e.g., brentuximab vedotin) is 0.4 mg/kg body weight. In other embodiments, the dosage of the anti-CD30 antibody drug-conjugate (e.g., brentuximab vedotin) is 0.5 mg/kg body weight. In other embodiments, the dosage of the anti-CD30 antibody drug-conjugate (e.g., brentuximab vedotin) is 0.6 mg/kg body weight. In other embodiments, the dosage of the anti-CD30 antibody drug-conjugate (e.g., brentuximab vedotin) is 0.7 mg/kg body weight. In other embodiments, the dosage of the anti-CD30 antibody drug-conjugate (e.g., brentuximab vedotin) is 0.8 mg/kg body weight. In other embodiments, the dosage of the anti-CD30 antibody drug-conjugate (e.g., brentuximab vedotin) is 0.9 mg/kg body weight. In other embodiments, the dosage of the anti-CD30 antibody drug-conjugate (e.g., brentuximab vedotin) is 1.0 mg/kg body weight. In other embodiments, the dosage of the anti-CD30 antibody drug-conjugate (e.g., brentuximab vedotin) is 1.1 mg/kg body weight. In other embodiments, the dosage of the anti-CD30 antibody drug-conjugate (e.g., brentuximab vedotin) is 1.2 mg/kg body weight. In other embodiments, the dosage of the anti-CD30 antibody drug-conjugate (e.g., brentuximab vedotin) is 1.3 mg/kg body weight. In other embodiments, the dosage of the anti-CD30 antibody drug-conjugate (e.g., brentuximab vedotin) is 1.4 mg/kg body weight. In other embodiments, the dosage of the anti-CD30 antibody drug-conjugate (e.g., brentuximab vedotin) is 1.5 mg/kg body weight. In other embodiments, the dosage of the anti-CD30 antibody drug-conjugate (e.g., brentuximab vedotin) is 1.6 mg/kg body weight. In other embodiments, the dosage of the anti-CD30 antibody drug-conjugate (e.g., brentuximab vedotin) is 1.7 mg/kg body weight. In other embodiments, the dosage of the anti-CD30 antibody drug-conjugate (e.g., brentuximab vedotin) is 1.8 mg/kg body weight. In other embodiments, the dosage of the anti-CD30 antibody drug-conjugate (e.g., brentuximab vedotin) is 1.9 mg/kg body weight. In other embodiments, the dosage of the anti-CD30 antibody drug-conjugate (e.g., brentuximab vedotin) is 2.0 mg/kg body weight. In other embodiments, the dosage of the anti-CD30 antibody drug-conjugate (e.g., brentuximab vedotin) is 2.1 mg/kg body weight. In other embodiments, the dosage of the anti-CD30 antibody drug-conjugate (e.g., brentuximab vedotin) is 2.2 mg/kg body weight. In other embodiments, the dosage of the anti-CD30 antibody drug-conjugate (e.g., brentuximab vedotin) is 2.3 mg/kg body weight. In other embodiments, the dosage of the anti-CD30 antibody drug-conjugate (e.g., brentuximab vedotin) is 2.4 mg/kg body weight. In other embodiments, the dosage of the anti-CD30 antibody drug-conjugate (e.g., brentuximab vedotin) is 2.5 mg/kg body weight. In other embodiments, the dosage of the anti-CD30 antibody drug-conjugate (e.g., brentuximab vedotin) is about 5 mg/kg body weight. In other embodiments, the dosage of the anti-CD30 antibody drug-conjugate (e.g., brentuximab vedotin) is about 10 mg/kg body weight.

In certain embodiments, an anti-CD30 antibody drug-conjugate (e.g., brentuximab vedotin) is administered at a flat dose. In some embodiments, the flat dose of the anti-CD30 antibody is a dose (e.g., flat dose) of at least about 1 to about 1500 mg, at least about 10 to about 1000 mg, such as, at least about 50 to about 800 mg, at least about 100 to about 600 mg, at least about 100 to about 400 mg or at least about 100 to about 200 mg, such as at least about 1 mg, at least about 3 mg, at least about 5 mg, at least about 8 mg, at least about 10 mg, at least about 20 mg, at least about 30 mg, at least about 40 mg, at least about 50 mg, at least about 60 mg, at least about 70 mg, at least about 80 mg, at least about 90 mg, at least about 100 mg, at least about 110 mg, at least about 120 mg, at least about 130 mg, at least about 140 mg, at least about 150 mg, at least about 160 mg, at least about 170 mg, at least about 180 mg, at least about 190 mg, at least about 200 mg, at least about 220 mg, at least about 240 mg, at least about 260 mg, at least about 280 mg, at least about 300 mg, at least about 320 mg, at least about 340 mg, at least about 360 mg, at least about 380 mg, at least about 400 mg, at least about 420 mg, at least about 440 mg, at least about 460 mg, at least about 480 mg, at least about 500 mg, at least about 600 mg, at least about 700 mg, at least about 800 mg, at least about 900 mg, at least about 1000 mg, at least about 1100 mg, at least about 1200 mg, at least about 1300 mg, at least about 1400 mg, or at least about 1500 mg.

In certain embodiments, an anti-CD30 antibody drug-conjugate described herein (e.g., brentuximab vedotin) is administered at a flat dose. In some embodiments, the flat dose of the anti-CD30 antibody drug-conjugate is a dose (e.g., flat dose) of about 1 to about 1500 mg, about 10 to about 1000 mg, such as, about 50 to about 800 mg, about 100 to about 600 mg, about 100 to about 400 mg or about 100 to about 200 mg, such as about 1 mg, about 3 mg, about 5 mg, about 8 mg, about 10 mg, about 20 mg, about 30 mg, about 40 mg, about 50 mg, about 60 mg, about 70 mg, about 80 mg, about 90 mg, about 100 mg, about 110 mg, about 120 mg, about 130 mg, about 140 mg, about 150 mg, about 160 mg, about 170 mg, about 180 mg, about 190 mg, about 200 mg, about 220 mg, about 240 mg, about 260 mg, about 280 mg, about 300 mg, about 320 mg, about 340 mg, about 360 mg, about 380 mg, about 400 mg, about 420 mg, about 440 mg, about 460 mg, about 480 mg, about 500 mg, about 600 mg, about 700 mg, about 800 mg, about 900 mg, about 1000 mg, about 1100 mg, about 1200 mg, about 1300 mg, about 1400 mg, or about 1500 mg.

An exemplary dosage regimen entails administration once per week, once about every 2 weeks, once about every 3 weeks, once about every 4 weeks, once about a month, once about every 3-6 months or longer. In certain embodiments, the anti-CD30 antibody drug-conjugate (e.g., brentuximab vedotin) is administered once about every 3 weeks.

In some embodiments, a subtherapeutic dose of an anti-CD30 antibody drug-conjugate (e.g., brentuximab vedotin) is used in the methods herein. The subtherapeutic dosages of an anti-CD30 antibody drug-conjugate (e.g., brentuximab vedotin) used in the methods herein are higher than 0.001 mg/kg and lower than 10 mg/kg. In some embodiments, the subtherapeutic dose is about 0.001 mg/kg-about 10 mg/kg, about 0.01 mg/kg-about 10 mg/kg, about 0.01 mg/kg-about 1 mg/kg, about 0.1 mg/kg-about 1 mg/kg, or about 0.001 mg/kg-about 0.1 mg/kg body weight. In some embodiments, the subtherapeutic dose is at least about 0.001 mg/kg, at least about 0.005 mg/kg, at least about 0.01 mg/kg, at least about 0.05 mg/kg, at least about 0.1 mg/kg, at least about 0.2 mg/kg, at least about 0.3 mg/kg, at least about 0.4 mg/kg, at least about 0.5 mg/kg, at least about 0.6 mg/kg, at least about 0.7 mg/kg, at least about 0.8 mg/kg, at least about 0.9 mg/kg, at least about 1 mg/kg, at least about 1.1 mg/kg, at least about 1.2 mg/kg, at least about 1.3 mg/kg, at least about 1.4 mg/kg, at least about 1.5 mg/kg, at least about 1.6 mg/kg, or at least about 1.7 mg/kg body weight.

In some embodiments, treatment is continued as long as clinical benefit is observed or until unacceptable toxicity or disease progression occurs.

Dosage and frequency vary depending on the half-life of the therapeutic agent (e.g., anti-CD30 antibody drug-conjugate) in the subject. In general, human antibodies show the longest half-life, followed by humanized antibodies, chimeric antibodies, and nonhuman antibodies. The dosage and frequency of administration can vary depending on whether the treatment is prophylactic or therapeutic. In prophylactic applications, a relatively low dosage is typically administered at relatively infrequent intervals over a long period of time. Some patients continue to receive treatment for the rest of their lives. In therapeutic applications, a relatively high dosage at relatively short intervals is sometimes required until progression of the disease is reduced or terminated, and until the patient shows partial or complete amelioration of symptoms of disease. Thereafter, the patient can be administered a prophylactic regime.

Actual dosage levels of the active ingredients in the pharmaceutical compositions of the present disclosure can be varied so as to obtain an amount of the active ingredient which is effective to achieve the desired therapeutic response for a particular patient, composition, and mode of administration, without being unduly toxic to the patient. The selected dosage level will depend upon a variety of pharmacokinetic factors including the activity of the particular compositions of the present disclosure employed, the route of administration, the time of administration, the rate of excretion of the particular compound being employed, the duration of the treatment, other drugs, compounds and/or materials used in combination with the particular compositions employed, the age, sex, weight, condition, general health, and prior medical history of the patient being treated, and like factors well known in the medical arts. A composition of the present disclosure can be administered via one or more routes of administration using one or more of a variety of methods well known in the art. As will be appreciated by the skilled artisan, the route and/or mode of administration will vary depending upon the desired results.

### IV. Articles of Manufacture or Kits

Also within the scope of the present disclosure provides an article of manufacture or kit which comprises a therapeutic agent described herein (e.g., an anti-CD30 antibody drug-conjugate). The article of manufacture or kit may further comprise instructions for use of the therapeutic agent (e.g., an anti-CD30 antibody drug-conjugate) in the methods of the invention. An article of manufacture or kit typically includes a label indicating the intended use of the contents of the article of manufacture or kit and instructions for use. The term label includes any writing, or recorded material supplied on or with the article of manufacture or kit. Thus, in certain embodiments, the article of manufacture or kit comprises instructions for the use of an anti-CD30 antibody drug-conjugate (e.g., brentuximab vedotin) in any of the methods disclosed herein such as in a method of decreasing the activity of CD30⁺ T regulatory (Treg) cells in a subject having cancer and/or in a method of increasing the ratio of CD8⁺ T cells to CD30⁺ T regulatory (Treg) cells in a subject having cancer. In some embodiments, the subject is a human.

Ins some embodiments, provided herein is an article of manufacture or kit for treating a subject afflicted with a cancer (e.g., having a cancer), the kit comprising: (a) a dosage ranging from about 0.1 mg to about 500 mg of an anti-CD30 antibody drug-conjugate; and (b) instructions for using the anti-CD30 antibody drug-conjugate in any of the methods disclosed herein. In certain embodiments for treating human patients, the article of manufacture or kit comprises an anti-human CD30 antibody drug-conjugate disclosed herein, e.g., brentuximab vedotin.

The article of manufacture or kit may further comprise a container. Suitable containers include, for example, bottles, vials (e.g., dual chamber vials), syringes (such as single or dual chamber syringes) and test tubes. The container may be formed from a variety of materials such as glass or plastic. The container holds the formulation.

The article of manufacture or kit may further comprise a label or a package insert, which is on or associated with the container, may indicate directions for reconstitution and/or use of the formulation. The label or package insert may further indicate that the formulation is useful or intended for subcutaneous, intravenous, or other modes of administration in an individual. The container holding the formulation may be a single-use vial or a multi-use vial, which allows for repeat administrations of the reconstituted formulation. The article of manufacture or kit may further comprise a second container comprising a suitable diluent. The article of manufacture or kit may further include other materials desirable from a commercial, therapeutic, and user standpoint, including other buffers, diluents, filters, needles, syringes, and package inserts with instructions for use.

In a specific embodiment, the present invention provides kits for a single dose-administration unit. Such kits comprise a container of an aqueous formulation of therapeutic antibody, including both single or multi-chambered pre-filled syringes. Exemplary pre-filled syringes are available from Vetter GmbH, Ravensburg, Germany.

The present invention also provides an anti-CD30 antibody drug-conjugate described herein that binds to CD30 (e.g., human CD30) in combination with one or more therapeutic agent (e.g., a second therapeutic agent) for us in any of the methods disclosed herein such as in a method of decreasing the activity of CD30⁺ T regulatory (Treg) cells in a subject having cancer and/or in a method of increasing the ratio of CD8⁺ T cells to CD30⁺ T regulatory (Treg) cells in a subject having cancer. In some embodiments, the article of manufacture or kit herein optionally further comprises a container comprising a second therapeutic medicament (e.g., a second therapeutic agent), wherein the anti-CD30 antibody drug-conjugate is a first medicament (e.g., a first therapeutic agent), and which article or kit further comprises instructions on the label or package insert for treating the individual with the second medicament, in an effective amount. In some embodiments, the kit further comprises an alkylating agent, an anthracycline, an antibiotic, an antifolate, an antimetabolite, an antitubulin agent, an auristatin, a chemotherapy sensitizer, a DNA minor groove binder, a DNA replication inhibitor, a duocarmycin, an etoposide, a fluorinated pyrimidine, a lexitropsin, a nitrosourea, a platinol, a purine antinetabolite, a puronycin, a radiation sensitizer, a steroid, a taxane, a topoisomerase inhibitor or a vinca alkaloid. In some embodiments, kit further comprises a second therapeutic agent is selected from the group consisting of adriamycin, an androgen, anthramycin (AMC), asparaginase, 5-azacytidine, azathioprine, bleomycin, busulfan, buthionine sulfoximine, camptothecin, carboplatin, carmustine (BSNU), CC-1065, chlorambucil, cisplatin, colchicine, cyclophosphanide, cytarabine, cytidine arabinoside, cytochalasin B, dacarbazine, dactinomycin (formerly actinomycin), daunorubicin, decarbazine, docetaxel, doxorubicin, an estrogen, 5-fluordeoxyuridine, 5-fluorouracil, granicidin D, hydroxydaunorubicin, hydroxyurea, idarubicin, ifosfarnide, irinotecan, loiustine (CCNU), nechloretharnine, melphalan, 6-mercaptopurine, mettotrexate, mithramycin, mitomycin C, mitoxanrone, nitroimidazole, paclitaxel, plicamycin, prednisone, prednisolone, procarbizine, streptozotocin, tenoposide, 6-thioguanine, thioTEPA, topotecan, vinblastine, vincristine, vinorelbine, VP-16 and VM-26. In some embodiments the kit comprises Cyclophospharnide, Doxorubicin, and Prednisone (CHP). In some embodiments the kit comprises Cyclophosphamide, Doxorubicin and Prednisolone. In some embodiments the kit comprises doxorubicin, vinblastine, and dacarbazine (AVD).

In another embodiment, provided herein is an article of manufacture or kit comprising the formulations described herein for administration in an auto-injector device. An auto-injector can be described as an injection device that upon activation, will deliver its contents without additional necessary action from the patient or administrator. They are particularly suited for self-medication of therapeutic formulations when the delivery rate must be constant and the time of delivery is greater than a few moments.

It is understood that the aspects and embodiments described herein are for illustrative purposes only and that various modifications or changes in light thereof will be suggested to persons skilled in the art and are to be included within the spirit and purview of this application and scope of the appended claims.

The invention will be more fully understood by reference to the following examples. They should not, however, be construed as limiting the scope of the invention. It is understood that the examples and embodiments described herein are for illustrative purposes only and that various modifications or changes in light thereof will be suggested to persons skilled in the art and are to be included within the spirit and purview of this application and scope of the appended claims.

### EXAMPLES

### Example 1. Anti-CD30 Antibody Drug-Conjugate Impairs Proliferating Inducible T Regulatory Cells In Vitro

CD4⁺ T cells, isolated from healthy donor PBMC (Astarte Biologics, Bothell Wash.) were used to generate inducible T regulatory cells (iTregs). iTreg differentiation was performed over 1-2 weeks in 6-well tissue culture plates at 37° C. Cells were incubated with CD3/CD28 MACS iBead particles (Miltenyi biotec) at a 1:32 bead/cell ratio, in 2-3 ml of X-VIVO 15 media (Lonza) containing IL-2 (50 ng/ml), TGFβ (50 ng/ml), and a 1:100 dilution of Lipid-Mixture 1 (Sigma-Aldrich). iTregs were evaluated for FoxP3 and CD30 expression by FACS analysis on an Attune NXT flow cytometer (Life Technologies). Following differentiation, individual donor iTreg populations ranged between 20-80% FoxP3⁺ and 40-70% CD30⁺.

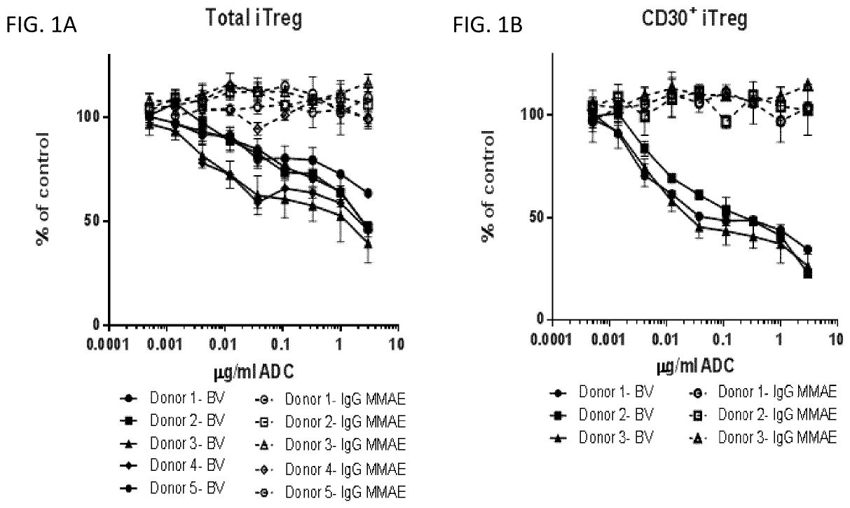

To evaluate the effect of brentuximab vedotin (BV), and anti-CD30 antibody drug-conjugate on iTreg viability, cells were driven to proliferate in vitro in the presence of BV or control antibody drug-conjugate (IgG-MMAE). Briefly, iTregs were mixed with CD3/CD28 beads (8:1) in RPMI 10% FCS and were distributed at approximately 2.0×10⁴ cells/well into a 96-well round-bottom plate. A titration of BV or control IgG-MMAE was added to replicate wells at the concentrations listed, for a final volume of 200 μl, and plates were incubated at 37° C. for 4-5 days. On the final day of the assay cells were stained with Zombie Aqua Viability Dye and a non-competing monoclonal αCD30-PE antibody (Biolegend) for FACS analysis. BV drove a dose-dependent reduction in total viable iTreg numbers from five separate donors (FIG. 1A). As populations of differentiated iTregs displayed heterogeneous CD30 expression, and BV selectively targets CD30 expressing cells, numbers of CD30⁺ iTregs were determined. Consistent with the loss of total iTregs from culture, BV showed enhanced depletion of CD30⁺ iTregs (FIG. 1B).

### Example 2. Treatment with an Anti-CD30 Antibody Drug-Conjugate Reduces iTreg Numbers, De-Repressing In Vitro CD8⁺ T Cell Proliferation

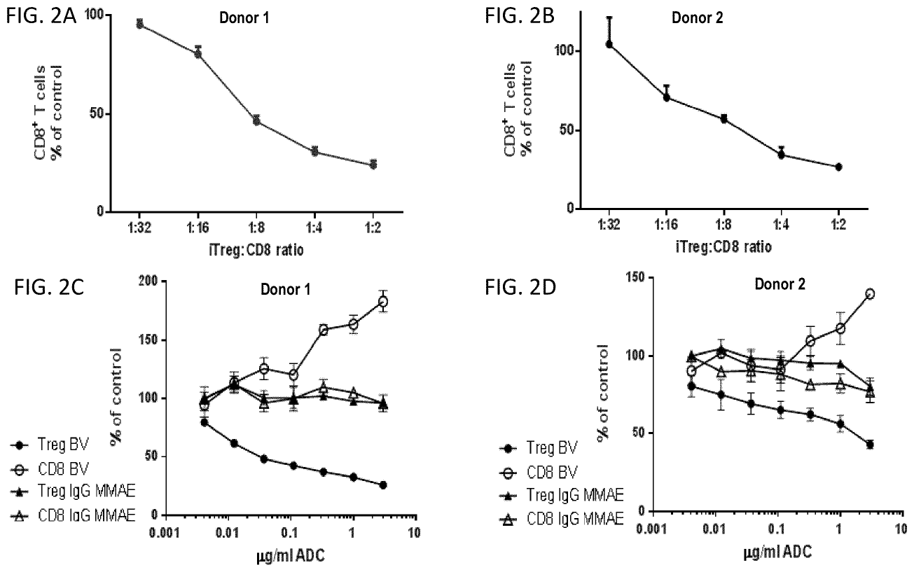

Addition of T regulatory cells to activated CD8⁺ T cells in vitro functionally suppresses CD8⁺ T cell proliferation. iTregs, generated as described above from two separate donors, showed suppressive activity on proliferating autologous CD8⁺ T cells in vitro. As shown in FIG. 2A and FIG. 2B, increasing the iTreg/CD8⁺ T cell ratio, for each donor, further abrogated T cell expansion, confirming suppressive function. To evaluate the effect of BV on iTreg suppression in vitro, co-culture suppression assays were performed. iTregs and CD8⁺ T cells were mixed at a 1:2 ratio and combined with CD3/CD28 beads. Replicate wells were treated with a titration of BV or control IgG-MMAE. After four days of culture, viable iTregs and CD8⁺ T cells were quantified by flow cytometry. As shown in FIG. 2C and FIG. 2D, increasing concentrations of BV selectively reduced iTregs, resulting in augmented CD8⁺ T cell accumulation for two separate donors.

### Example 3. An Anti-CD30 Antibody Drug-Conjugate Depletes Naturally-Occurring Peripheral Blood Tregs, but not CD8⁺ T Cells In Vitro

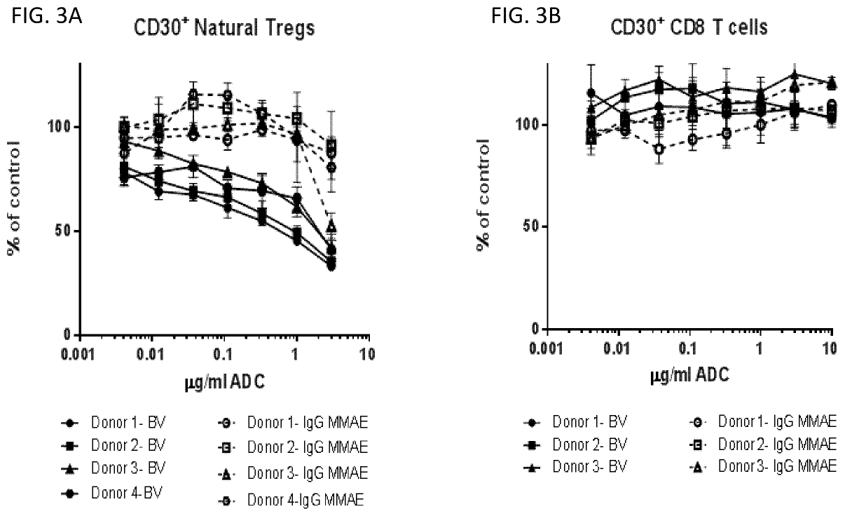

CD25^{hi} CD127^{lo} T regulatory cells or CD8⁺ T cells, enriched from peripheral blood derived leukoreduction system (LRS) chambers, were plated with CD3/CD28 beads+IL-2 in round-bottom 96-well tissue culture plates for 4-5 days with a titration of BV or control IgG-MMAE. On the final day of the assay, cells were stained as described above (viability dye and αCD30-PE antibody) and evaluated by flow cytometry. For each donor, >50% of activated peripheral blood Tregs and CD8⁺ T cells expressed CD30, demonstrating abundant antigen expression for BV targeting. As with iTreg cultures, BV drove a dose-dependent reduction of viable CD30⁺ Treg numbers from four separate donors (FIG. 3A). In contrast, up to 10% g/ml of BV did not deplete CD30⁺ CD8⁺ T cells (FIG. 3B).

### Example 4. An Anti-CD30 Antibody Drug-Conjugate Reduces Human T Regulatory Cells and Increases CD8/Treg Ratio in a Xeno-GVHD Model

To evaluate activity of BV on activated human T cell subtypes in vivo, a model of acute xenograft-driven graft-versus-host disease (xeno-GVHD) was employed. In this model, immune deficient NSG mice are lightly irradiated (2Gy) on day 0 followed by adoptive transfer of 5×10⁶ healthy donor PBMC on day 1. Disease course is driven by activation and proliferation of mouse-reactive human CD4⁺ and CD8⁺ T cells, and disease kinetics are slowed by addition of human T regulatory cells.

To evaluate the effect of BV on activated CD8⁺ T cells and Tregs in vivo, Xeno-GVHD mice received a single i.p. injection of PBS or BV (3 mg/kg) in PBS on day 5. On day 12, spleens were harvested and manually dissociated through a 70% m cell strainer. Following centrifugation, individual spleens were resuspended in 3 ml of ACK lysis buffer (Sigma) for 3 minutes to remove red blood cells. Cells were washed with RPMI+10% FCS to stop the RBC lysis reaction. Spleen cells were resuspended in 4 mls of media and 200 μl of the cell suspension was used for staining and analysis by flow cytometry (FACS). Spleen cell suspensions were stained with Zombie Aqua Viability Dye (Biolegend) followed by staining with fluorescently labeled antibodies targeting human CD3, CD8, CD4, FoxP3, CD25, CD45, and murine CD45.1 (1:50 dilution, Biolegend) in staining buffer (PBS, 2% FCS, 1% NRS, 0.05% NaN₃) at 4° C. for 30 minutes. Cells were washed and resuspended in 120%1 of staining buffer for plate-based FACS using an Attune NXT flow cytometer. All events were collected from 80%1 of sample, and FACS-measured cell concentrations were used to calculate numbers of human immune cells. CD8⁺ T cells were identified as viability dye^{neg}, hCD45⁺, mCD45.1⁻, CD3⁺, CD8⁺ cells. Tregs were identified as viability dye^{neg}, hCD45⁺, mCD45.1⁻, CD3⁺, CD4⁺, FoxP3⁺, CD25⁺ cells.

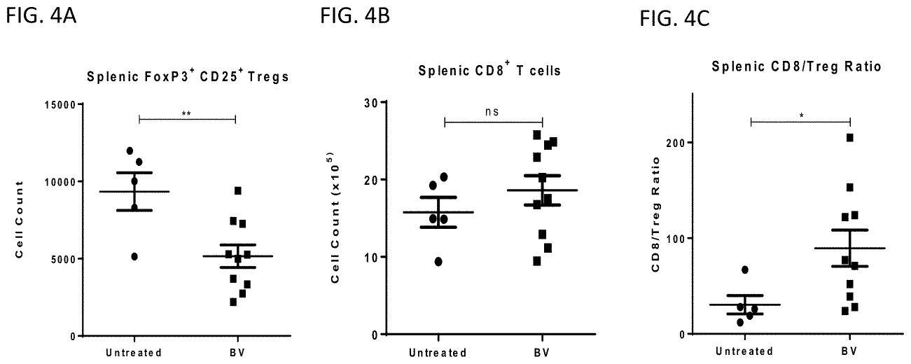

As shown in FIG. 4A, BV significantly reduced human T regulatory cells in the spleen compared to PBS alone. In contrast, splenic CD8⁺ T cells were unaffected by BV treatment with a trend toward increased numbers (FIG. 4B). Taken together, BV treatment increased the CD8⁺ T cell/Treg ratio in vivo consistent with heightened cytotoxic T cell activity (FIG. 4C).

### Example 5. An Anti-CD30 Antibody Drug-Conjugate Reduced CD30⁺ T Regulatory Cells in Patients with Classical Hodgkin Lymphoma

The effects of BV on circulating immune cells has not previously been fully elucidated. Sixty-two patients adult patients with classical Hodgkin lymphoma (cHL) that had relapsed or was refractory to frontline chemotherapy were enrolled in a study to evaluate treatment with BV. Patients were excluded if they previously received prior salvage therapy, including salvage radiotherapy, for refractory cHL; BV or any immuno-oncology therapy affecting the PD-1, CTLA4, or CD137 pathways; and/or allogeneic or autologous stem cell transplant (ASCT). BV was administered to the patients at a dose of 1.8 mg/kg on Day 1 and the patients were assessed on Day 8. Immunophenotyping of peripheral blood by flow cytometry was performed by Q2 Solutions on heparinized whole blood. Cell pellets, resulting from plasma banking, were sent to Adaptive for T Cell Receptor β (TCRβ) sequencing. Peripheral blood mononuclear cells were isolated from CPT tubes, frozen, and then analyzed in batches by Caprion using their intracellular cytokine staining platform following peptide stimulation.

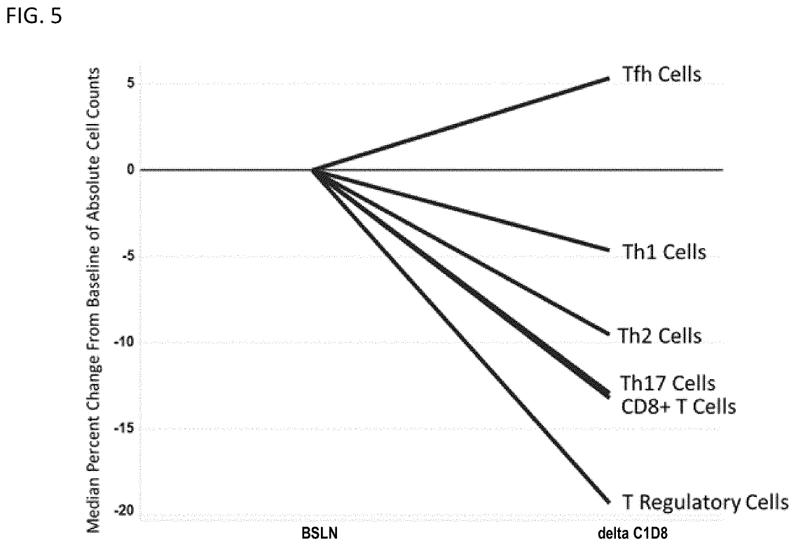

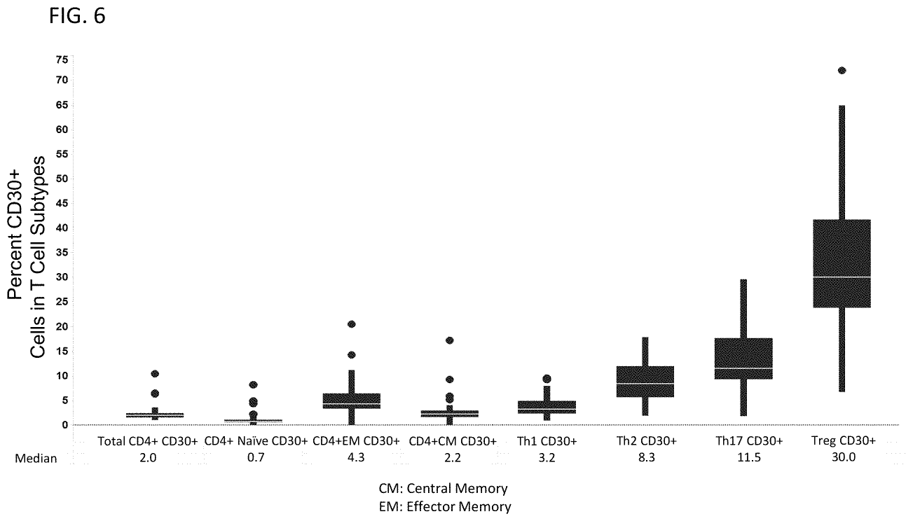

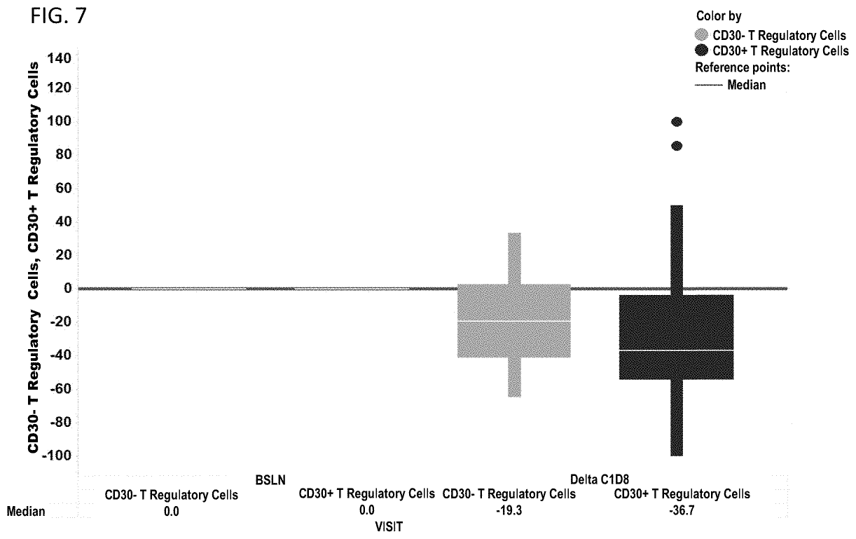

BV treatment appears resulted in the reduction of T helper cell subset populations including regulatory T cells (FIG. 5). As BV is a CD30-targeted therapeutic, and as CD30 is expressed transiently on immune cells including B and T cells, CD30 expression on peripheral blood cells in the patients was evaluated. Regulatory T cells expressed more CD30 than any other T cell subset examined (FIG. 6). These CD30-expressing regulatory T cells were significantly reduced in number in the peripheral blood after treatment with BV (FIG. 7). In addition, BV treatment resulted in the reduction of CD30⁺ regulatory T cells more significantly when compared to the reduction of other CD30⁺ T helper cells.

### Example 6. CD30 Expression is Enriched on CD25^{hi} CCR4^{hi} FOXP3^{hi} Effector Tregs in PBMC

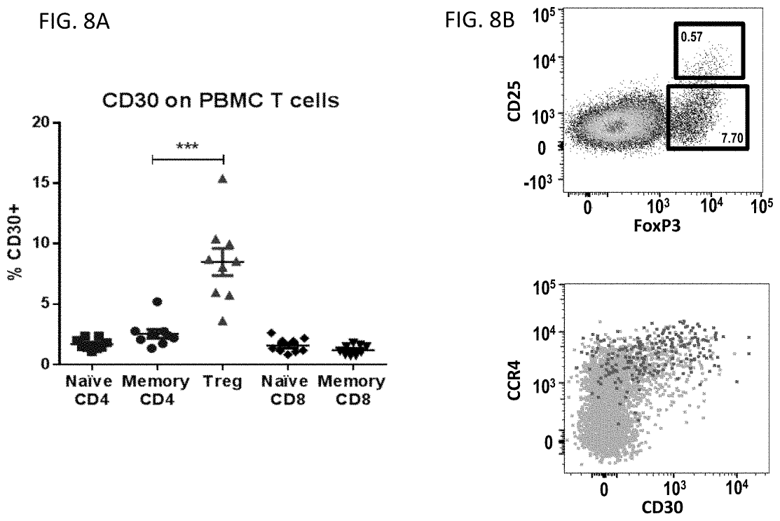

Cryopreserved PBMCs from healthy donors were stained with viability dye, anti-CD3, CD4, CD8, CD45RA, FoxP3, CCR4, CD127 and CD25 (1:50 dilution, Biolegend) and evaluated by flow cytometry. Memory and naïve T cell populations were discriminated by CD45RA expression. T regulatory cells were identified by appropriate expression of CD4, CD25, FoxP3 and/or CD127. As shown in FIG. 8A, CD30 is most frequently expressed by T regulatory cells compared to CD4⁺ and CD8⁺ memory and naïve T cell subsets. Furthermore, subdividing FoxP3-expressing Tregs into CD25^{hi} and CD25^{low/neg} populations showed expression of CD30 is highly associated with the effector T regulatory subset (FoxP3^{hi} CD25^{hi} CCR4^{hi}) (FIG. 8B).

### Example 7. Differences in T Cell Subset CD30 Expression and Drug Efflux May Underlie Sensitivity to BV

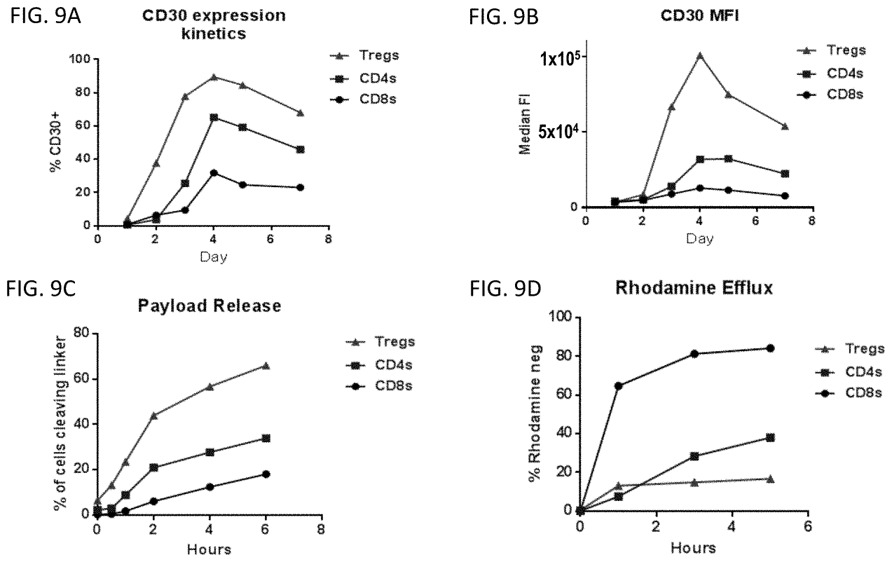

T cell subsets (CD4⁺, CD8⁺, CD4⁺ CD25⁺ CD127⁻) were sorted from cryopreserved PBMC by magnetic selection and activated with CD3/CD28 beads (1:4) in vitro for 7 days. Each day, CD30 expression was monitored by flow cytometry. Values from a representative donor are shown as the proportion of cells expressing CD30 (FIG. 9A) and the relative magnitude of expression by Mean Fluorescence Intensity (MFI) (FIG. 9B). Enriched T regulatory cells displayed heightened CD30 expression kinetics and overall CD30 receptor levels compared to CD4⁺ and CD8⁺ T cells following activation.

To examine whether heightened CD30 expression on T regulatory cells translated into enhanced payload delivery, an internalization assay was performed. On day 4 of in vitro stimulation, at peak receptor expression, T cell subsets were incubated with a conditionally fluorescent anti-CD30 mAb for 6 hours. Along the incubation time-course, cells were analyzed by flow cytometry for intracellular payload release via activation of a quenched-fluorescent reporter-CD30 mAb construct (FIG. 9C). T regulatory cells showed accelerated and increased release of fluorescent payload relative to CD4⁺ and CD8⁺ T cells, consistent with heightened CD30 expression at day 4. These data support the conclusion that differences in CD30 expression may facilitate enhanced drug delivery to T regulatory cells (Tregs).

Sensitivity of cells to many chemotherapies, including MMAE, is influenced by cell-intrinsic drug efflux activity. T cell subsets were evaluated for relative efflux pump activity using a rhodamine 123 efflux assay, following manufacturer's protocol (Chemicon International, Multidrug Resistance Direct Dye Efflux Assay). Enriched T cell populations were loaded with rhodamine 123, incubated in a 37° C. water bath, and were measured for loss of fluorescence over a 5-hour time-course by flow cytometry. T regulatory cells showed the slowest rhodamine-123 efflux among T cell subsets while CD8⁺ T cells showed rapid clearance of intracellular rhodamine-123 (FIG. 9D).

Altogether, activated T regulatory cells demonstrate heightened CD30 receptor expression and payload delivery, along with impaired drug efflux capacity, providing mechanistic rationale for the observed sensitivity to BV relative to cytotoxic CD8⁺ T cells.

1. A method of decreasing the activity of CD30⁺ T regulatory (Treg) cells in a subject comprising administering to the subject an antibody-drug conjugate, wherein the antibody-drug conjugate comprises an anti-CD30 antibody or an antigen-binding portion thereof conjugated to a monomethyl auristatin.

2. The method of claim 1, wherein decreasing the activity of CD30⁺ Treg cells comprises a decrease in the number of CD30⁺ Treg cells.

3. The method of claim 2, wherein the number of CD30⁺ Treg cells is decreased relative to the number of one or more other types of CD4⁺ T cells.

4. The method of claim 3, wherein the one or more other types of CD4⁺ T cells comprise Th1 cells, Th2 cells or Th17 cells.

5. The method of claim 4, wherein the one or more other types of CD4⁺ T cells comprise Th1 CD30⁺ cells, Th2 CD30⁺ cells or Th17 CD30⁺ cells.

6. The method of claim 5, wherein the number of CD30⁺ Treg cells is decreased relative to the number of CD30⁺ Treg cells in the subject prior to administration of the antibody-drug conjugate.

7. The method of claim 1, wherein decreasing the activity of CD30⁺ Treg cells comprises a decrease in the function of CD30⁺ Treg cells.

8. The method of claim 7, wherein the decrease in the function of CD30⁺ Treg cells is relative to the function of CD30⁺ Treg cells in a subject prior to administration of the antibody-drug conjugate.

9. A method of increasing the ratio of CD8⁺ T cells to CD30⁺ T regulatory (Treg) cells in a subject having cancer comprising administering to the subject an antibody-drug conjugate, wherein the antibody-drug conjugate comprises an anti-CD30 antibody or an antigen-binding portion thereof conjugated to a monomethyl auristatin.

10. A method of modulating the immune response in a subject having cancer comprising administering to the subject an antibody-drug conjugate, wherein the antibody-drug conjugate comprises an anti-CD30 antibody or an antigen-binding portion thereof conjugated to a monomethyl auristatin, wherein the modulation comprises increasing the ratio of CD8⁺ T cells to CD30⁺ T regulatory (Treg) cells in the subject.

11. The method of claim 9, wherein the ratio of CD8⁺ T cells to CD30⁺ Treg cells is increased relative to the ratio of CD8⁺ T cells to CD30⁺ Treg cells in the subject prior to the administration of the antibody-drug conjugate.

12. The method of claim 1, wherein the CD30⁺ Treg cells are CD30⁺ inducible T regulatory (iTreg) cells or CD30⁺ peripheral T regulatory (pTreg) cells.

13. The method of claim 1, wherein the monomethyl auristatin is monomethyl auristatin E (MMAE).

14. The method of claim 1, wherein the monomethyl auristatin is monomethyl auristatin F (MMAF).

15-16. (canceled)

17. The method of claim 1, wherein the anti-CD30 antibody of the antibody-drug conjugate comprises a heavy chain variable region and a light chain variable region, wherein the heavy chain variable region comprises:
(i) a CDR-H1 comprising the amino acid sequence of SEQ ID NO: 1;
(ii) a CDR-H2 comprising the amino acid sequence of SEQ ID NO: 2; and
(iii) a CDR-H3 comprising the amino acid sequence of SEQ ID NO: 3; and
wherein the light chain variable region comprises:
(i) a CDR-L1 comprising the amino acid sequence of SEQ ID NO: 4;
(ii) a CDR-L2 comprising the amino acid sequence of SEQ ID NO: 5; and
(iii) a CDR-L3 comprising the amino acid sequence of SEQ ID NO: 6.

18. The method of claim 17, wherein the anti-CD30 antibody of the antibody-drug conjugate comprises a heavy chain variable region comprising the amino acid sequence of SEQ ID NO: 7 and a light chain variable region comprising the amino acid sequence of SEQ ID NO: 8.

19-43. (canceled)

44. The method of claim 1, wherein the method further comprises administering one or more additional therapeutic agents capable of modulating the immune response.

45. The method of claim 44, wherein the one or more additional therapeutic agents is not an antibody or antigen-binding fragment thereof.

46. The method of claim 44, wherein the one or more additional therapeutic agents is an antibody or antigen-binding fragment thereof.

47. The method of claim 1, wherein the method further comprises administering one or more additional therapeutic agents.

48-62. (canceled)
